(19) United States
(12) Patent Application Publication
(10) Pub. No.: US 2024/0175007 A1
(43) Pub. Date: May 30, 2024

(54) COMPOSITIONS FOR AND METHODS OF IMPROVING DIRECTED EVOLUTION OF BIOMOLECULES

(71) Applicant: THE GEORGE WASHINGTON UNIVERSITY, Washington, DC (US)

(72) Inventors: Erik Ali RODRIGUEZ, Washington, DC (US); Sara MATTSON, Washington, DC (US); Felipe MONTECINOS-FRANJOLA, Washington, DC (US); Nicholas CSIKAI, Washington, DC (US); Leena ZITOUN, Washington, DC (US)

(21) Appl. No.: 18/429,316

(22) Filed: Jan. 31, 2024

Related U.S. Application Data
(63) Continuation of application No. PCT/US22/81694, filed on Dec. 15, 2022.
(60) Provisional application No. 63/290,024, filed on Dec. 15, 2021.

(51) Int. Cl. C12N 15/10; C12N 9/12; C12N 15/86

(52) U.S. Cl. CPC C12N 15/1058; C12N 9/1252; C12N 15/86

(57) ABSTRACT

Disclosed herein are compositions for and methods of directing evolution of biomolecules. The disclosed methods simplify traditional error prone PCR of a single or multiple genes expressed on plasmid DNA for directed evolution and remove several previously required molecular biology steps, thereby affording facile purification of plasmid DNA expressing biomolecules directly from mammalian cells.

### I. CROSS-REFERENCE TO RELATED APPLICATIONS

This application is a continuation of PCT Application No. PCT/US2022/081694, filed 15 Dec. 2022, which claims priority to U.S. Provisional Application No. 63/290,024 filed 15 Dec. 2021, each of which is incorporated herein in its entirety.

### II. REFERENCE TO THE SEQUENCE LISTING

The Sequence Listing submitted 31 Jan. 2024 as a .xml file named “GW099 (091019-709897)—Sequence Listing”, created on 31 Jan. 2024 and having a size of 3 kilobytes, is hereby incorporated by reference pursuant to 37 C.F.R. § 1.52(e)(5).

### III. BACKGROUND

The process of optimizing the properties of biological molecules is paramount for many industrial and medical applications. Directed evolution is a powerful technique for modifying and improving biomolecules such as proteins or nucleic acids (DNA or RNA). Mimicking the mechanism of natural evolution, one can enhance a desired property by applying a suitable selection pressure and sorting improved variants. Thus, directed evolution and methods to evolve biomolecules are essential to the research and development of synthetic biology tools to locate and treat disease, safely synthesize complex molecules, and remove plastic, carbon, or carbon dioxide from the environment.

The experiments provided herein describe a new directed evolution of biomolecules that (i) is extremely quick, (ii) safer than viral methods, and (iii) requires less hands-on experimental time. Prior to the disclosed invention, researchers typically used error prone PCR, mutation introducing primers, DNA recombination, somatic hypermutation in B lymphocytes, viruses, to incorporate somatic hypermutation into yeast and other organisms. Viral directed evolution is potentially hazardous with viruses that infect mammalian cells and users. Current error prone PCR of a gene and screening in bacteria takes longer with many molecular biology steps.

Thus, there remains an unmet need for developing faster and more effective methods of directed evolution of one or more biomolecules of interest. The novel and inventive approach described herein simplified the traditional error prone PCR by removing several molecular biology steps to meet that need.

### IV. BRIEF DESCRIPTION OF THE FIGURES

FIG. 1 shows the comparison of fluorescent protein evolution schemes. On the left side, traditional bacterial evolution takes ˜14 hours of hands-on experiment time. On the right, as described herein, the novel single PCR MEGA directed evolution of biomolecules takes ˜4 hours of hands-on experiment time. The new, single PCR MEGA directed evolution of biomolecules is four times faster with fewer steps for less errors. PCR means polymerase chain reaction, Dephos. means dephosphorylation, and FACS is fluorescence-activated cell sorting.

FIG. 2 shows a schematic of primer design, error prone PCR, and circularization in E. coli. On the left, FIG. 2 shows a schematic of the pDuEx2 plasmid. Primers were placed before the CMV promoter to ensure transcription and translation of Gene 1 and Gene 2. Error prone PCR introduces mutations in the DNA sequence that result in amino acid changes during translation. The PCR amplified the entire plasmid to create a linear plasmid with a double stranded DNA break at the exact location with 3′-Adenine overhangs from the Taq DNA polymerase. Removal of 3′-Adenine overhangs is performed with T4 DNA Polymerase to enhance circularization. The single PCR is only possible with a new Taq DNA polymerase fused to a double stranded DNA binding protein to amplify the entire plasmid without proofreading. Template circular plasmid DNA is methylated and removed by DpnI digestion. The linear plasmid DNA is circularized in E. coli. The circular plasmid DNA is lipofected into mammalian cells and purified.

FIG. 3 shows the evolution of a new class of fluorescent protein without added biliverdin. The fluorescence-activated cell sorting (FACS) histogram is shown. Solid black is HEK293 cells that do not express a fluorescent protein and show the autofluorescence distribution in the wild-type HEK293A cells. Red and Blue were new fluorescent protein variants expressed in HEK293 cells after one and two rounds of directed evolution, respectively. The single PCR MEGA directed evolution of biomolecules works to create new fluorescent proteins from non-fluorescent proteins.

FIG. 4 shows single cell selection of new smURFP variants expressed in human (HEK293A) cells without added biliverdin. The FACS histogram was shown. Black was HEK293A cells without a fluorescent protein. Blue was the original template, smURFP. Orange and red were smURFP Mutant and smURFP Mutant Library, respectively. smURFP Mutant 10-10 is 240% brighter than smURFP in HEK293A cells without added biliverdin.

FIG. 5 shows fluorescence screening of unique variants in cells. HEK293A cells were lipofected with smURFP and variant DNA. 1 μM BVMe₂ was added to smURFP 24 hours after transfection to find the maximal fluorescence. 48 hours after lipofection, the average fluorescence intensity was calculated from 25 cells. Each well was normalized to fluorescence of smURFP without BVMe₂. smURFP Mutant 10-10 was 240% brighter than smURFP. Other variants were <1.5-fold relative to smURFP.

### V. BRIEF SUMMARY

Disclosed herein is a system for use in a disclosed method of directed evolution.

Disclosed herein is a system comprising a single PCR mammalian expression generated array for the directed evolution of biomolecules.

Disclosed herein is a system comprising a single PCR mammalian expression generated array for the directed evolution of biomolecules, wherein the use of viral vectors or viruses is avoided.

Disclosed herein is a system for directed evolution of a biomolecule of interest, the system comprising a DNA polymerase fused to a DNA binding protein, wherein the DNA polymerase lacks editing activity; and a plasmid comprising (i) a promoter for transcription and translation, and (ii) a polynucleotide sequence encoding a biomolecule of interest to be evolved, wherein the system evolves the biomolecule of interest by introducing one or more mutations into the polynucleotide sequence encoding the biomolecule of interest.

Disclosed herein is a system for directed evolution of one or more biomolecules of interest, the system comprising a DNA polymerase fused to a DNA binding protein, wherein the DNA polymerase lacks editing activity; and a plasmid comprising one or more promoters for transcription and translation, and one or more polynucleotide sequences encoding one or more biomolecules of interest to be evolved, wherein the system evolves the one or more biomolecules of interest by introducing one or more mutations into the one or more polynucleotide sequences encoding the one or more biomolecules of interest.

Disclosed herein is a kit comprising any disclosed component, ingredient, instruments, and/or devices for use in a disclosed method of directed evolution. Disclosed herein is kit comprising a DNA polymerase fused to a DNA binding protein, wherein the DNA polymerase does not comprise any editing activity; a host-specific plasmid comprising at least one promoter for transcription and translation; and at least one reaction solution. Disclosed herein is a kit for a single PCR mammalian expression generated array for the directed evolution of biomolecules. Disclosed herein is a kit for a single PCR mammalian expression generated array for the directed evolution of biomolecules, wherein the use of viral vectors or viruses is avoided.

Disclosed herein is method of directed evolution of at least one biomolecule of interest to be evolved, the method comprising preparing a circular plasmid DNA comprising at least one promoter for transcription and translation, and a polynucleotide sequence encoding the at least one biomolecule of interest to be evolved; amplifying the entire circular plasmid DNA into an entire linear plasmid DNA by performing a single error prone PCR in the presence of a DNA polymerase fused to a DNA binding protein, wherein the DNA polymerase does not comprise any editing activity; evolving the at least one biomolecule of interest by introducing mutations into the polynucleotide sequence encoding the at least one biomolecule of interest during the single error prone PCR by addition of increased concentrations of magnesium, manganese, and deoxynucleotide triphosphates during the PCR amplification of the entire circular plasmid DNA into linear plasmid DNA containing mutations throughout the DNA sequence; circularizing the linear plasmid DNA into a circular plasmid DNA in bacteria; incorporating the circular plasmid DNA into a host cell; assessing at least one evolved biomolecule for one or more resultant activities of interest in the host cell.

Disclosed herein is method of directed evolution of at least one biomolecule of interest to be evolved, the method comprising preparing a circular plasmid DNA comprising at least one promoter for transcription and translation, and a polynucleotide sequence encoding the at least one biomolecule of interest to be evolved; amplifying the entire circular plasmid DNA into an entire linear plasmid DNA by performing a single error prone PCR in the presence of a DNA polymerase fused to a DNA binding protein, wherein the DNA polymerase does not comprise any editing activity; evolving the at least one biomolecule of interest by introducing mutations into the polynucleotide sequence encoding the at least one biomolecule of interest during the single error prone PCR by addition of increased concentrations of magnesium, manganese, and deoxynucleotide triphosphates during the PCR amplification of the entire circular plasmid DNA into linear plasmid DNA containing mutations throughout the DNA sequence; circularizing the linear plasmid DNA into a circular plasmid DNA in bacteria; incorporating the circular plasmid DNA into a host cell; assessing at least one evolved biomolecule for one or more resultant activities of interest in the host cell, wherein the method does not comprise use of a virus or viral vector.

Disclosed herein is a method for directed evolution, the method comprising preparing DNA library by performing error prone PCR mutagenesis; circularizing plasmid DNA by electroporating linear plasmid DNA in bacteria; screening bacteria and purifying circular plasmid DNA; growing mammalian cells; electroporating mammalian cells with circular plasmid DNA; subjecting the mammalian cells to cell sorting; lysing the mammalian cells and recovering circular plasmid DNA; electroporating bacteria and plating electroporated bacteria; lipofecting circular plasmid DNA into mammalian cells and screening lipofected mammalian cells; lipofecting circular DNA into mammalian cells and screening lipofected mammalian cells; and identifying amino acid mutations by sequencing and analyzing DNA.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Biomolecular Evolution

Biomolecular evolution can be described as a path from one functional biomolecule to another in the space of all possible biomolecular variants, where each variant has an assigned fitness. In nature, the fitness is the ability of an organism to reproduce in a particular environment and consequently spread its genes. In the laboratory, the selective pressure and therefore the fitness are set by the experimenter. Directed evolution is a growing field in synthetic biology and has the capacity to provide new proteins or nucleic acids on the basis of predefined industrial or biomedical needs. It relies on the Darwinian principle of mutation and selection, where the probability of success is determined by the ability to find rare, optimal variants within a large pool of sequences. All directed evolution experiments require a measurable activity that acts as a fitness indicator to drive the selection process “uphill” within the protein or nucleic acid sequence space, ultimately resulting in a biomolecule with a desired set of properties. For instance, the Examples set forth below describe selection of increased fluorescence produced by fluorescent proteins as the fitness indicator.

### B. Error Prone PCR

Error prone PCR is a method by which random mutants are created by changing wild-type nucleotide to one of the other three nucleotides on DNA. The technique is based on the well-founded PCR (polymerase chain reaction), which is a standard technique in many molecular biology laboratories. Normally the replication of DNA by the polymerase is extremely specific, the difference in error prone PCR is that the fidelity of the Taq DNA polymerase is modulated by alteration of the composition of the reaction buffer. In these conditions, the polymerase makes mistakes in the base paring during DNA synthesis that results in the introduction of errors in the newly synthesized complementary DNA strand. By carefully controlling the buffer composition the frequency of mis-incorporation of nucleotide bases, and therefore the number of errors introduced into the sequence may be regulated. In directed evolution experiments, the substitution frequency is normally controlled at around 1-3 base pair substitutions per kilobase of DNA. For the technique to work properly, it is important to use a Taq DNA polymerase which does not have proof-reading ability. This proof-reading, or auto-correction of nucleotide sequence, is a property that is found in many commercially available DNA polymerases. However, use of a proof-reading DNA polymerase is used in an error prone PCR reaction will result in the automatic correction of the mismatched nucleotides, and any mutations that were introduced during the reaction will be lost.

### C. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, “derived from” can mean “derived from”, “based on”, “obtained from”, “obtained from” or “isolated from” depending on the context. For example, in an aspect, the term “derived from” can mean that an amino acid sequence is derived from the parent amino acid sequence by introducing a modification to at least one position. Thus, the derived amino acid sequence differs from the corresponding parent amino acid sequence in at least one corresponding position (numbering based on Kabat's EU index numbering system for the antibody Fc region). In an aspect, a disclosed amino acid sequence derived from the parent amino acid sequence can differ by 1 to 15 amino acid residues at corresponding positions. In an aspect, a derived amino acid sequence can have a high degree sequence identity to its parent amino acid sequence. In an aspect, a disclosed amino acid sequence derived from the parent amino acid sequence can have at least 50%, at least 60%, at least 70%, at least 80%, at least 90%, or more sequence identity to the parent or original sequence.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing a disease or disorder (e.g., a disease or disorder that can be treated, prevented, and/or managed by one or more disclosed evolved biomolecules). In an aspect, a subject can have a disease or disorder, be suspected of having a disease or disorder, or be at risk of developing a disease or disorder that can be treated, prevented, and/or managed by one or more disclosed evolved biomolecules.

As used herein, the term “diagnosed” means having been subjected to an examination by a person of skill, for example, a physician, and found to have a condition that can be diagnosed or treated by one or more of the disclosed evolved biomolecules or more of the disclosed methods. For example, “diagnosed with a disease or disorder” means having been subjected to an examination by a person of skill, for example, a physician, and found to have a condition (such as a disease or disorder) that can be treated by one or more of the disclosed evolved biomolecules or by one or more of the disclosed methods. For example, “suspected of having a disease or disorder” can mean having been subjected to an examination by a person of skill, for example, a physician, and found to have a condition (such as a disease or disorder that can be treated, prevented, and/or managed by one or more disclosed evolved biomolecules) that can likely be treated by one or more of by one or more of the disclosed evolved biomolecules or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a disease or disorder (e.g., a disease or disorder that can be treated, prevented, and/or managed by one or more disclosed evolved biomolecules). In an aspect, a patient can refer to a subject that has been diagnosed with or is suspected of having a disease or disorder. In an aspect, a patient can refer to a subject that has been diagnosed with or is suspected of having a disease or disorder and is seeking treatment or receiving treatment for a disease or disorder that can be treated, prevented, and/or managed by one or more disclosed evolved biomolecules.

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., a disease or disorder that can be treated, prevented, and/or managed by one or more disclosed evolved biomolecules) based upon an earlier diagnosis by a person of skill and thereafter subjected to treatment for the disease or disorder that can be treated, prevented, and/or managed by one or more disclosed evolved biomolecules. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder that can be treated, prevented, and/or managed by one or more disclosed evolved biomolecules). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having disease or disorder that can be treated, prevented, and/or managed by one or more disclosed evolved biomolecules). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as a disease or disorder that can be treated, prevented, and/or managed by one or more disclosed evolved biomolecules). For example, treating a disease or disorder can reduce one or more symptoms of a disease or disorder in a subject by 1%-100% as compared to a control (such as, for example, an individual not having a disease or disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder. It is understood that treatment does not necessarily refer to a cure or complete ablation or eradication of a disease or disorder. However, in an aspect, treatment can refer to a cure or complete ablation or eradication of a disease or disorder. As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as a disease or disorder that can be treated, prevented, and/or managed by one or more disclosed evolved biomolecules) or a related complication from progressing to that complication.

As used herein, the terms “administering” and “administration” refer to any method of providing one or more of the disclosed polynucleotides, disclosed evolved biomolecules, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, optic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a one or more of the disclosed polynucleotides, disclosed evolved biomolecules, or a combination thereof can comprise administration directly into the CNS or the PNS. Administration can be continuous or intermittent. Administration can comprise a combination of one or more route.

In an aspect, the skilled person can determine an efficacious dose, an efficacious schedule, and an efficacious route of administration for one or more one or more of the disclosed polynucleotides, disclosed evolved biomolecules, or a combination thereof to treat and/or prevent and/or manage a disease or disorder (such as a disease or disorder that can be treated, prevented, and/or managed by one or more disclosed evolved biomolecules). In an aspect, the skilled person can also alter, change, or modify an aspect of an administering step to improve efficacy of the one or more of the disclosed polynucleotides, disclosed evolved biomolecules.

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

The term “determining” as used, for example, in a phrase such as, e.g., “determining whether a cell has a desired property” refers to observing, measuring, testing, characterizing, and/or otherwise evaluating a target (such as, e.g., a cell, or polypeptide, or nucleic acid) in order to detect whether the target exhibits or possesses as particular property, feature, and/or characteristic. In an aspect, whether a biomolecule has evolved can be determined.

The term “selecting” as used, for example, in a phrase such as “selecting a cell that exhibits a desired property” refers to choosing and/or segregating (e.g., physically separating and/or isolating) a target (such as, e.g., a cell, a polypeptide, or a nucleic acid) in order to identify and/or collect those targets that exhibits or possesses a particular property, a particular feature, a particular characteristic, or any combination thereof.

As used herein, “modifying the method” can comprise modifying or changing one or more features or aspects of one or more steps of a disclosed method. For example, in an aspect, a method can be altered by changing the amount of one or more of the disclosed polynucleotides, disclosed evolved biomolecules, or a combination thereof administered to a subject, or by changing the frequency of administration of one or more of the disclosed polynucleotides, disclosed evolved biomolecules, or a combination thereof administered to a subject, by changing the duration of time the one or more of the disclosed polynucleotides, disclosed evolved biomolecules, or a combination thereof are administered to a subject, or by substituting for one or more of the disclosed components and/or reagents with a similar or equivalent component and/or reagent. The same applies to all disclosed components/ingredients/instruments.

As used herein, the term “pharmaceutically acceptable carrier” refers to sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

The term “contacting” as used herein refers to bringing one or more of the disclosed polynucleotides, disclosed evolved biomolecules, or a combination thereof together with a target area or intended target area in such a manner that the one or more of the disclosed polynucleotides, disclosed evolved biomolecules, or a combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by a disease or disorder (such as a disease or disorder that can be treated and/or prevented and/or managed by a disclosed evolved biomolecule). In an aspect, a target area or intended target area can be any organ, tissue, or cells that are affected by a disease or disorder (such as a disease or disorder that can be treated and/or prevented and/or managed by a disclosed evolved biomolecule).

As used herein, “determining” can refer to measuring or ascertaining the presence and severity of a disease or disorder, such as, for example, a disease or disorder that can be treated, prevented, and/or managed by one or more disclosed evolved biomolecules. Methods and techniques used to determine the presence and/or severity of a disease or disorder are typically known to the medical arts. For example, the art is familiar with the ways to identify and/or diagnose the presence, severity, or both of a disease or disorder (such as, for example, a disease or disorder that can be treated and/or prevented and/or managed by a disclosed evolved biomolecule).

As used herein, “effective amount” and “amount effective” can refer to an amount that is sufficient to achieve the desired result such as, for example, the treatment and/or prevention of a disease or disorder (e.g., a disease or disorder that can be treated and/or prevented and/or managed by a disclosed evolved biomolecule) or a suspected disease or disorder. As used herein, the terms “effective amount” and “amount effective” can refer to an amount that is sufficient to achieve the desired an effect on an undesired condition (e.g., a disease or disorder that can be treated and/or prevented and/or managed by a disclosed evolved biomolecule). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed polynucleotide, a disclosed evolved biomolecule, or a combination thereof that (i) treats the particular disease, condition, or disorder (e.g., a disease or disorder that can be treated and/or prevented and/or managed by a disclosed evolved biomolecule), (ii) attenuates, ameliorates, or eliminates one or more symptoms of the particular disease, condition, or disorder e.g., a genetic disease or disorder that can be treated and/or prevented and/or managed by a disclosed evolved biomolecule), or (iii) delays the onset of one or more symptoms of the particular disease, condition, or disorder described herein (e.g., a disease or disorder that can be treated and/or prevented and/or managed by a disclosed evolved biomolecule). The specific therapeutically effective dose level for any particular patient will depend upon a variety of factors including the disorder being treated and the severity of the disorder; the disclosed polynucleotide, the disclosed evolved biomolecule, or the combination thereof employed; the disclosed methods employed; the age, body weight, general health, sex and diet of the patient; the time of administration; the route of administration; the rate of excretion of the disclosed polynucleotide, the disclosed evolved biomolecule, or the combination thereof employed; the duration of the treatment; drugs used in combination or coincidental with the disclosed isolated nucleic acid molecules, disclosed vectors, or disclosed pharmaceutical formulations employed, and other like factors well known in the medical arts. For example, it is well within the skill of the art to start doses of the disclosed polynucleotide, the disclosed evolved biomolecule, or the combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed polynucleotide, the disclosed evolved biomolecule, or the combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder that can be treated and/or prevented and/or managed by a disclosed evolved biomolecule.

The terms “nucleic acid,” “nucleic acid molecule,” “polynucleotide,” and the like refers to a deoxyribonucleotide or ribonucleotide polymer in either single-stranded or double-stranded form, and, unless specifically indicated otherwise, encompasses polynucleotides containing known analogs of native sequence nucleotides that can function in a similar manner as native sequence nucleotides. It will be understood that when a nucleic acid molecule is represented by a DNA sequence, this also includes RNA molecules having the corresponding RNA sequence in which “U” (uridine) replaces “T” (thymidine).

Reference to a nucleic acid or a polynucleotide “encoding” a polypeptide means that, upon transcription of the polynucleotide and translation of the mRNA produced therefrom, a polypeptide is produced. The encoding polynucleotide is considered to include both the coding strand, whose nucleotide sequence is identical to an mRNA, as well as its complementary strand. It will be recognized that such an encoding polynucleotide is considered to include degenerate nucleotide sequences, which encode the same amino acid residues. Nucleotide sequences encoding a polypeptide can include polynucleotides containing introns as well as the encoding exons.

The term “polypeptide” or “protein” refers to a polymer of two or more amino acid residues. The terms apply to amino acid polymers in which one or more amino acid residue is an artificial chemical analogue of a corresponding native sequence amino acid, as well as to native sequence amino acid polymers. The term “recombinant protein” or “recombinant polypeptide” refers to a protein that is produced by expression of a nucleotide sequence encoding the amino acid sequence of the protein from a recombinant DNA molecule.

As used herein, the abbreviations for the genetically encoded L-enantiomeric amino acids used in the disclosure methods are conventional and are as follows in Table 1. amino acids used in the disclosure methods are conventional and are as follows in Table 1.

TABLE 1
Amino Acid Abbreviations

| Amino Acid | 1-Letter | 3-Letter | Amino Acid | 1-Letter | 3-Letter |
|---|---|---|---|---|---|
| Alanine | A | Ala | Leucine | L | Leu |
| Arginine | R | Arg | Lysine | K | Lys |
| Asparagine | N | Asn | Methionine | M | Met |
| Aspartic acid | D | Asp | Phenylalanine | F | Phe |
| Cysteine | C | Cys | Proline | P | Pro |
| Glutamine | Q | Gln | Serine | S | Ser |
| Glutamic acid | E | Glu | Threonine | T | Thr |
| Glycine | G | Gly | Tryptophan | W | Trp |
| Histidine | H | His | Tyrosine | Y | Tyr |
| Isoleucine | I | Ile | Valine | V | Val |

“Hydrophilic Amino Acid” refers to an amino acid exhibiting a hydrophobicity of less than zero according to the normalized consensus hydrophobicity scale of Eisenberg et al., 1984, J. Mol. Biol. 179: 125-142. Genetically encoded hydrophilic amino acids include Thr (T), Ser (S), His (H), Glu (E), Asn (N), Gln (Q), Asp (D), Lys (K) and Arg (R).

“Acidic Amino Acid” refers to a hydrophilic amino acid having a side chain pKa value of less than 7. Acidic amino acids typically have negatively charged side chains at physiological pH due to loss of a hydrogen ion. Genetically encoded acidic amino acids include Glu (E) and Asp (D).

“Basic Amino Acid” refers to a hydrophilic amino acid having a side chain pKa value of greater than 7. Basic amino acids typically have positively charged side chains at physiological pH due to association with hydrogen ion. Genetically encoded basic amino acids include His (H), Arg (R) and Lys (K).

“Polar Amino Acid” refers to a hydrophilic amino acid having a side chain uncharged at physiological pH, but which has at least one bond in which the pair of electrons shared in common by two atoms is held more closely by one of the atoms. Genetically encoded polar amino acids include Asn (N), Gln (Q), Ser (S) and Thr (T).

“Hydrophobic Amino Acid” refers to an amino acid exhibiting a hydrophobicity of greater than zero according to the normalized consensus hydrophobicity scale of Eisenberg, 1984, J. Mol. Biol. 179:125-142. Exemplary hydrophobic amino acids include Ile (I), Phe (F), Val (V), Leu (L), Trp (W), Met (M), Ala (A), Gly (G), Tyr (Y), Pro (P), and proline analogues.

“Aromatic Amino Acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C1-C6) alkyl, substituted (C1-C6) alkyl, (C1-C6) alkenyl, substituted (C1-C6) alkenyl, (C1-C6) alkynyl, substituted (C1-C6) alkynyl, (C1-C21)) aryl, substituted (C5-C20) aryl, (C6-C26) alkaryl, substituted (C6-C26) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

“Nonpolar Amino Acid” refers to a hydrophobic amino acid having a side chain uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic Amino Acid” refers to a hydrophobic amino acid having an aliphatic hydrocarbon side chain. Genetically encoded aliphatic amino acids include Ala (A), Val (V), Leu (L) and Ile (I).

The term “non-naturally” with regard to amino acids can include any amino acid molecule not included as one of the 20 amino acids listed in Table 1 above as well as any modified or derivatized amino acid known to one of skill in the art. Non-naturally amino acids can include but are not limited to β-alanine, α-amino butyric acid, γ-amino butyric acid, γ-(aminophenyl) butyric acid, α-amino isobutyric acid, ϵ-amino caproic acid, 7-amino heptanoic acid, β-aspartic acid, aminobenzoic acid, aminophenyl acetic acid, aminophenyl butyric acid, γ-glutamic acid, cysteine (ACM), ϵ-lysine, methionine sulfone, norleucine, norvaline, ornithine, d-ornithine, p-nitro-phenylalanine, hydroxy proline, 1,2,3,4,-tetrahydroisoquinoline-3-carboxylic acid, and thioproline.

The term “conservatively modified variation,” when used in reference to a particular polynucleotide sequence, refers to different polynucleotide sequences that encode identical or essentially identical amino acid sequences, or where the polynucleotide does not encode an amino acid sequence, to essentially identical sequences. Because of the degeneracy of the genetic code, a large number of functionally identical polynucleotides encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleotide sequence variations are “silent variations,” which can be considered a species of “conservatively modified variations.” As such, it will be recognized that each polynucleotide sequence disclosed herein as encoding a fluorescent protein variant also describes every possible silent variation. It will also be recognized that each codon in a polynucleotide, except AUG, which is ordinarily the only codon for methionine, and UUG, which is ordinarily the only codon for tryptophan, can be modified to yield a functionally identical molecule by standard techniques. Accordingly, each silent variation of a polynucleotide that does not change the sequence of the encoded polypeptide is implicitly described herein. Furthermore, it will be recognized that individual substitutions, deletions, or additions that alter, add, or delete a single amino acid or a small percentage of amino acids (typically less than 5%, and generally less than 1%) in an encoded sequence can be considered conservatively modified variations, provided alteration results in the substitution of an amino acid with a chemically similar amino acid.

As to amino acid sequences, one of skill will recognize that individual substitutions, deletions or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

Conservative amino acid substitutions providing functionally similar amino acids are well known in the art. Dependent on the functionality of the particular amino acid, i.e., catalytically important, structurally important, sterically important, different groupings of amino acid may be considered conservative substitutions for each other. Table 2 provides groupings of amino acids that are considered conservative substitutions based on the charge and polarity of the amino acid, the hydrophobicity of the amino acid, the surface exposure/structural nature of the amino acid, and the secondary structure propensity of the amino acid.

TABLE 2
Groupings of Conservative Amino Acid Substitutions
based on Functionality of the Residue

| Important Feature | Conservative Groupings |
|---|---|
| Charge/Polarity | H, R, and K |
| | D and E |
| | C, T, S, G, N, Q, and Y |
| | A, P, M, L, I, V, F, and W |
| Hydrophobicity | D, E, N, Q, R, and K |
| | C, S, T, P, G, H, and Y |
| | A, M, I, L, V, F, and W |
| Structural/Surface Exposure | D, E, N, Q, H, R, and K |
| | C, S, T, P, A, G, W, and Y |
| | M, I, L, V, and F |
| Secondary Structure | A, E, Q, H, K, M, L, and R |
| Propensity | C, T, I, V, F, Y, and W |
| | S, G, P, D, and N |
| Evolutionary Conservation | D and E |
| | H, K, and R |
| | N and Q |
| | S and T |
| | L, I, and V |
| | F, Y, and W |
| | A and G |
| | M and C |

Two or more amino acid sequences or two or more nucleotide sequences are considered to be “substantially identical” or “substantially similar” if the amino acid sequences or the nucleotide sequences share at least 80% sequence identity with each other, or with a reference sequence over a given comparison window. Thus, substantially similar sequences include those having, for example, at least 85% sequence identity, at least 90% sequence identity, at least 95% sequence identity, or at least 99% sequence identity. In certain embodiments, substantially similar sequences will have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity.

A subject nucleotide sequence is considered “substantially complementary” to a reference nucleotide sequence if the complement of the subject nucleotide sequence is substantially identical to the reference nucleotide sequence. The term “stringent conditions” refers to a temperature and ionic conditions used in a nucleic acid hybridization reaction. Stringent conditions are sequence dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature, under defined ionic strength and pH, at which 50% of the target sequence hybridizes to a perfectly matched probe.

The term “allelic variants” refers to polymorphic forms of a gene at a particular genetic locus, as well as cDNAs derived from mRNA transcripts of the genes, and the polypeptides encoded by them. The term “preferred mammalian codon” refers to the subset of codons from the set of codons encoding an amino acid that are most frequently used in proteins expressed in mammalian cells as chosen from the following list: Gly (GGC, GGG); Glu (GAG); Asp (GAC); Val (GUG, GUC); Ala (GCC, GCU); Ser (AGC, UCC); Lys (AAG); Asn (AAC); Met (AUG); Ile (AUC); Thr (ACC); Trp (UGG); Cys (UGC); Tyr (UAU, UAC); Leu (CUG); Phe (UUC); Arg (CGC, AGG, AGA); Gln (CAG); His (CAC); and Pro (CCC).

As used herein, a diabody is a noncovalent dimer of single-chain Fv (scFv) fragment that consists of the heavy chain variable (VH) and light chain variable (VL) regions connected by a small peptide linker. Another form of diabody is single-chain (Fv)2 in which two scFv fragments are covalently linked to each other.

As used herein, a triabody or trimeric antibody is built of three single chain pairs of the variable heavy and light chain regions of antibody (e.g., a trimer of a fragment of an immunoglobulin).

The terms “actively inducing,” “actively induced,” “active induction,” and the like, as applied to expression of a polypeptide or of nucleic acid message encoding a polypeptide, indicate an active step taken by an actor, such as a person, to induce the subject expression. For example, providing doxycycline to a cell comprising a doxycycline-dependent promoter to induce expression of message encoding a polypeptide, so as to induce expression of that polypeptide, is actively induced expression of that polypeptide. However, the constitutive expression of a polypeptide, that is not the result of addition of additional factors or of activation of an inducible promoter, is not actively induced expression.

As used herein, random mutagenesis is a powerful tool for generating enzymes, proteins, entire metabolic pathways, or even entire genomes with desired or improved properties. This technology is used to evolve genes in vitro through an iterative process consisting of recombinant generation.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. In an aspect, the term cationic and ionizable in the context of LNP lipids can be used interchangeably, e.g., wherein ionizable lipids are cationic depending on the pH.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition in a disclosed method.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence, but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

In an aspect, a disclosed polynucleotide can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

As used herein, “CRISPR” or “clustered regularly interspaced short palindromic repeat” is an ideal tool for correction of genetic abnormalities as the system can be designed to target genomic DNA directly. A CRISPR system involves two main components—a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8α2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicellulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Staphylococcus Aureus or Streptococcus pyogenes.

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein, such as for example, Staphylococcus Aureus or Streptococcus pyogenes, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein, or an effector domain fused with a CRISPR/Cas protein.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed polynucleotide, a disclosed evolved biomolecule, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a disease or disorder that can be treated and/or prevented and/or managed by a disclosed evolved biomolecule.

Disclosed are the components to be used to prepare the disclosed polynucleotides, the disclosed evolved biomolecules, or the combinations thereof as well as the disclosed polynucleotides, the disclosed evolved biomolecules, or the combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### A. Compositions for Use in the Disclosed Methods

### 1. System for Directed Evolution

Disclosed herein is a system for use in a disclosed method of directed evolution. Disclosed herein is a system comprising a single PCR mammalian expression generated array for the directed evolution of biomolecules. Disclosed herein is a system comprising a single PCR mammalian expression generated array for the directed evolution of biomolecules, wherein the use of viral vectors or viruses is avoided.

Disclosed herein is a system for directed evolution of a biomolecule of interest, the system comprising a DNA polymerase fused to a DNA binding protein, wherein the DNA polymerase lacks editing activity; and a plasmid comprising (i) a promoter for transcription and translation, and (ii) a polynucleotide sequence encoding a biomolecule of interest to be evolved, wherein the system evolves the biomolecule of interest by introducing one or more mutations into the polynucleotide sequence encoding the biomolecule of interest.

Disclosed herein is a system for directed evolution of one or more biomolecules of interest, the system comprising a DNA polymerase fused to a DNA binding protein, wherein the DNA polymerase lacks editing activity; and a plasmid comprising one or more promoters for transcription and translation, and one or more polynucleotide sequences encoding one or more biomolecules of interest to be evolved, wherein the system evolves the one or more biomolecules of interest by introducing one or more mutations into the one or more polynucleotide sequences encoding the one or more biomolecules of interest.

In an aspect, a disclosed system can evolve at least one biomolecule of interest by introducing one or more mutations into the polynucleotide sequence encoding at least one biomolecule of interest and circularizing the linear plasmid DNA by electroporating the linear plasmid DNA in bacteria. In an aspect, a disclosed circular plasmid DNA comprises a bacterial library. In an aspect, a disclosed system can prepare a DNA library for next generation sequencing.

In an aspect, a disclosed system can comprise one or more disclosed components, ingredients, instruments, and/or devices for use to perform the experiments described in, for instance, Example 1, Example 2, Example 3, Example 4, Example 5, Example 6, Example 7, Example 8, Example 9, Example 10, Example 11, or any combination thereof.

In an aspect, a disclosed system can comprise one or more disclosed components, ingredients, instruments, and/or devices for use to perform one or more time in the experiments described in, for instance, Example 1, Example 2, Example 3, Example 4, Example 5, Example 6, Example 7, Example 8, Example 9, Example 10, Example 11, or any combination thereof.

In an aspect, a disclosed system can be used to (i) generate a library containing sufficient gene variants, and (ii) perform a high-throughput screening of library members with the desired and/or resulting properties and/or activities.

In an aspect of a disclosed system, a disclosed DNA polymerase can lack editing activity. In an aspect of a disclosed system, a disclosed entire plasmid can be amplified by the DNA polymerase fused to a DNA binding protein. In an aspect of a disclosed system, a disclosed DNA polymerase fused to a DNA binding protein can amplify at least about 0.1 kilobases of DNA. In an aspect of a disclosed system, a disclosed DNA polymerase fused to a DNA binding protein can amplify about 0.1 kilobases of DNA to about 15 kilobases of DNA.

In an aspect of a disclosed system, a disclosed DNA polymerase fused to a DNA binding protein can amplify more than about 15 kilobases of DNA. In an aspect of a disclosed system, a disclosed DNA polymerase fused to a DNA binding protein can amplify more than about 15 kilobases of DNA after optimization of at least one aspect of the system wherein at least one aspect of the system can comprise reaction temperature, extension time, number of cycles, amount of DNA primers, or any combination thereof.

In an aspect of a disclosed system, a disclosed plasmid can comprise two biomolecules of interest to be evolved. In an aspect of a disclosed system, a disclosed plasmid can comprise at least two or more biomolecules of interest to be evolved. wherein the biomolecule of interest can comprise a nucleic acid, a oligonucleotide, a oligonucleoside, a protein, a peptide, an enzyme, an amino acid, a lipid, an organic molecule, a small molecule, or any combination thereof.

In an aspect of a disclosed system, a disclosed molecule of interest can comprise a silencing RNA or a guide RNA. In an aspect of a disclosed system, a disclosed silencing RNA can be expressed to knock down gene expression and/or increase the expression of a protein library to identify one or more phenotypes.

In an aspect of a disclosed system, a disclosed silencing RNA can be expressed to knock down gene expression and/or increase the expression of a protein of interest. For example, in an aspect of a disclosed system, a disclosed silencing RNA can be expressed to knock down gene expression and/or increase the expression of a protein implicated in a disease or disorder. Genes that are implicated in a disease or disorder are known to the art and can include ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1 L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANE1, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6 L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREB1 L, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12 L, MED13, MED13 L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1 L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAI1, RALGAPA1, RANBP2, RB1CC1, RELN, RERE, REV3 L, RIC1, RIMS1, RIMS2, RNF213, ROBO1, ROBO2, ROBO3, ROS1, RP1, RP1 L1, RTTN, RUSC2, RYR1, RYR2, SACS, SAMD9, SAMD9 L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1 L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect of a disclosed system, a disclosed silencing RNA can be expressed to knock down gene expression and/or increase the expression of a protein implicated in a disease or disorder with a gene product that is directly or indirectly linked to one or more genetic diseases. Such genes include but are not limited to the following: dystrophin including mini- and micro-dystrophins (DMD); titin (TTN); titin cap (TCAP) α-sarcoglycan (SGCA), β-sarcoglycan (SGCB), γ-sarcoglycan (SGCG) or δ-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); β-galactosidase (GLB1); β-glucuronidase (GUSB); hyaluronoglucosaminidase 1 (HYAL1); acid ceramidase (ASAHI); galactosylceramidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase α (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase α class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel β subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); carnitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic β1 subunit (CFHRNB1); cholinergic receptor nicotinic β2 subunit (CFHRNB2); cholinergic receptor nicotinic β3 subunit (CHRNB3); cholinergic receptor nicotinic β4 subunit (CFHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin α (DTNA); protein kinase AMP-activated, noncatalytic γ2 (PRKAG2); cysteine- and glycine-rich protein 3 (CSRP3); vinculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cb1D type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histidine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβR1); transforming growth factor β receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2).

In an aspect of a disclosed system, a disclosed guide RNA can be expressed for genomic DNA editing in a library of CRISPR/CasX mutants. CRISPR and Cas are discussed supra. In an aspect of a disclosed system, a disclosed RNA aptamer can be selected for binding to a protein expressed on the same plasmid.

In an aspect of a disclosed system, a disclosed protein can comprise a protein endogenous to a host cell, a protein exogenous to a host cell, or any combination thereof. In an aspect of a disclosed system, a disclosed protein endogenous to a host cell, a protein exogenous to a host cell, or any combination thereof can increase the number of small molecules including inorganic salts in a cell, excreted by a cell, outside of a cell, or any combination thereof.

In an aspect, a disclosed small molecule can refer to any organic or inorganic material that is not a polymer. In an aspect of a disclosed system, a disclosed small molecule can exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a disclosed small molecule can be a drug that can enter cells easily because it has a low molecular weight. In an aspect, a disclosed small molecule can be used in conjunction with a disclosed system, a disclosed kit, and/or a disclosed method.

In an aspect of a disclosed system, a disclosed small molecule can comprise one or more inorganic salts. In an aspect of a disclosed system, a disclosed organic molecule can comprise organic molecules native to a host cell, organic molecules not native to a host cell, or any combination thereof. In an aspect of a disclosed system, a disclosed organic molecule can comprise an intracellular organic molecule, an extracellular organic molecule, or any combination thereof. In an aspect, a disclosed organic molecule can comprise an amino acid. Amino acids are discussed supra.

In an aspect, a disclosed biomolecule of interest can comprise an antisense oligonucleotide (ASO), a small interfering RNA (siRNA), a silencing RNA, a DNA aptamer, RNA aptamer, a transfer RNA (tRNA), a ribosomal RNA (rRNA), a long non-coding RNA (lncRNA), a microRNAs (miRNAs), a small nucleolar RNA (snoRNA), a small nuclear RNA (snRNAs), a piwi-interacting RNA (piRNA), a messenger RNA (mRNA), a gene-editing guide RNA (gRNA), or any combination thereof.

In an aspect of a disclosed system, a disclosed biomolecule of interest can comprise a full-length antibody, an antigen binding fragment of a full-length antibody, a Fab fragment, a single chain antibody (scFv), a diabody, a triabody, a minibody, a nanobody, a single-domain antibody, camelids, or any combination thereof.

In an aspect of a disclosed system, a disclosed biomolecule of interest can comprise an enzyme that can perform novel protein, RNA, and/or DNA modifications, or can perform a combination of novel protein, RNA, and/or DNA modifications.

In an aspect of a disclosed system, a disclosed biomolecule of interest can comprise an enzyme that can elevate native small molecules. In an aspect of a disclosed system, a disclosed biomolecule of interest can comprise an enzyme that can create novel small molecules not typically or not normally present inside a specific cell (such as, for example, a mammalian cell or a bacterial cell).

In an aspect of a disclosed system, a disclosed biomolecule of interest can comprise an enzyme that can degrade plastic or waste products, thereby forming useful molecules. In an aspect of a disclosed system, a disclosed biomolecule of interest comprises an enzyme degrade carbon dioxide or other gases to create synthetically useful molecules and reduce greenhouse gases. In an aspect of a disclosed system, a disclosed biomolecule of interest can comprise an enzyme that can degrade plastic or waste products, thereby forming useful molecules, can degrade carbon dioxide or other gases to create synthetically useful molecules and reduce greenhouse gases, or any combination thereof. In an aspect, a disclosed system can be used in metabolomic engineering. In an aspect, a disclosed system can be used to create one or more synthetic organisms for production of unnatural amino acids, wherein one or more unnatural amino acids can be used for creation of one or more proteins with new functions. In an aspect of a disclosed system, a disclosed system can be used to produce one or more biological circuits for molecule sensing and memory storage, genetic reprogramming, or any combination thereof. In an aspect, a disclosed plasmid can be any plasmid having one or more protomers for transcription and translation. In an aspect, a disclosed plasmid can be any plasmid known to the art.

In an aspect of a disclosed system, a disclosed biomolecule of interest can be used in or relevant to the agricultural arts. In an aspect of a disclosed system, a disclosed biomolecule of interest can be used to improve a property and/or characteristic and/or aspect of a crop. As known to those skilled in the art, a crop can be a food crop, a feed crop, a fiber crop, and oil crop, an ornamental crop, and/or an industrial crop. For example, in an aspect of a disclosed system, a disclosed biomolecule of interest can improve disease resistance and/or can increase or enhance immunity to pests and/or pathogens (including for example, fungi, fungal-like organisms, bacteria, mollicutes, phytoplasmas, viruses, viroids, nematodes, parasitic higher plants, or any combination thereof) in a crop of interest or a crop having commercial, economic, and/or social value. In an aspect of a disclosed system, a disclosed biomolecule of interest can improve yield of a crop of interest crop of interest or a crop having commercial, economic, and/or social value. In an aspect of a disclosed system, a disclosed biomolecule of interest can increase or enhance tolerance to abiotic stress in a crop of interest crop of interest or a crop having commercial, economic, and/or social value. In an aspect of a disclosed system, a disclosed biomolecule of interest can increase or enhance tolerance to drought of a crop of interest crop of interest or a crop having commercial, economic, and/or social value. In an aspect of a disclosed system, a disclosed biomolecule of interest can increase or enhance resistance to one or more herbicides in a crop of interest or a crop having commercial, economic, and/or social value. In an aspect of a disclosed system, a disclosed biomolecule of interest can increase or enhance photosynthesis or the rate of photosynthesis in a crop of interest or a crop having commercial, economic, and/or social value. In an aspect of a disclosed system, a disclosed biomolecule of interest can improve the biomass of a crop of interest or a crop having commercial, economic, and/or social value. In an aspect of a disclosed system, a disclosed biomolecule of interest can increase and/or enhance the nutritional content of a crop of interest or a crop having commercial, economic, and/or social value. In an aspect of a disclosed system, a disclosed biomolecule of interest can improve the tolerance to temperature, ultraviolet radiation, or other abiotic factors of a crop of interest or a crop having commercial, economic, and/or social value. In an aspect of a disclosed system, a disclosed biomolecule of interest can be a fungus (e.g., an entomopathogenic fungi) that can be evolved as a pesticide. In an aspect of a disclosed system, the directed evolution of one or more biomolecule of interest that improve resistance to pests and/or pathogens can be genetically engineered into a crop of interest or a crop having commercial, economic, and/or social value. In an aspect of a disclosed system, the directed evolution of one or more biomolecule of interest can be a pheromone of a pest and/or a pathogen that infests and/or damages a crop of interest or a crop having commercial, economic, and/or social value. In an aspect of a disclosed system, an evolved pheromone can be applied and/or contacted with a crop of interest or a crop having commercial, economic, and/or social value. In an aspect, a disclosed crop of interest or a disclosed crop having commercial, economic, and/or social value can be any crop including, but not limited to rice, maize, wheat, soybean, potatoes, tomatoes, sugarcane, grapes, cotton, apples, onions, sorghums, cucumbers, garlic, bananas, watermelons, mangos, guavas, rapeseed, chilis, peppers, olives, eggplants, oranges, sweet potatoes, barleys, sunflowers, tangerines, mandarins, clementine, yams, cabbages, spinaches, strawberries, peaches, nectarines, tobaccos, coffees, teas, lettuces, peas, blueberries, pumpkins, cassavas, and combinations thereof.

In an aspect, a disclosed plasmid can be a bacterial expression plasmid, a mammalian expression plasmid, an insect expression plasmid, a viral expression plasmid, a species-specific host organism expression plasmid, or any combination thereof.

In an aspect, a disclosed system can comprise a mutation efficiency of at least about 1-in-400 base pairs, about 1-in-350 base pair, 1-in-300 base pairs, 1-in-250 base pairs, 1-in-200 base pairs, 1-in-150 base pairs, 1-in-100 base pairs. In an aspect, a disclosed system can comprise a mutation efficiency of about 1-in-200 base pairs.

In an aspect, a disclosed system can comprise a plasmid comprising the one or more promoters for transcription and translation (either in a mammalian or a non-mammalian cell). Promoters are known to the art. Depending on the level and tissue-specific expression desired, a variety of promoter elements can be used. In an aspect of a disclosed system, a disclosed promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced. Tissue-specific promoters are known to the art and include, but are not limited to, neuron-specific promoters, muscle-specific promoters, liver-specific promoters, skeletal muscle-specific promoters, and heart-specific promoters. Liver-specific promoters” are known to the art and include, but are not limited to, the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bA1b) promoter, the murine albumin (mA1b) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter.

Ubiquitous/constitutive promoters are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vaculating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

Inducible promoters are known to the art and can be regulated by positive or negative control. Factors that can regulate an inducible promoter include, but are not limited to, chemical agents (e.g., the metallothionein promoter or a hormone inducible promoter), temperature, and light.

In an aspect of a disclosed system, a disclosed host cell can comprise a bacterial cell, a yeast cell, an insect cell, a mammalian cell, a species-specific host cell, or any combination thereof, wherein the host cell comprises a disclosed plasmid. In an aspect, a disclosed host cell comprising a disclosed plasmid can be a human cell. In an aspect of a disclosed system, a disclosed host cell can comprise a bacterial cell, a yeast cell, an insect cell, a mammalian cell, a species-specific host cell, or any combination thereof. In an aspect, a disclosed host cell can be a human cell.

### 2. Kits

Disclosed herein is a kit comprising any disclosed component, ingredient, instruments, and/or devices for use in a disclosed method of directed evolution. Disclosed herein is kit comprising a DNA polymerase fused to a DNA binding protein, wherein the DNA polymerase does not comprise any editing activity; a host-specific plasmid comprising at least one promoter for transcription and translation; and at least one reaction solution. Disclosed herein is a kit for a single PCR mammalian expression generated array for the directed evolution of biomolecules. Disclosed herein is a kit for a single PCR mammalian expression generated array for the directed evolution of biomolecules, wherein the use of viral vectors or viruses is avoided.

In an aspect, a disclosed reaction solution can comprise PCR buffers, nuclease-free water, and various concentrations of magnesium, manganese, and deoxynucleotide triphosphates, or any combination thereof. In an aspect, a disclosed host-specific plasmid can comprise a bacterial expression plasmid, a mammalian expression plasmid, an insect expression plasmid, a viral expression plasmid, or another host organism expression plasmid. In an aspect, a disclosed can further comprise chemically or electro-competent bacterial cells for the circularization of linear plasmid DNA libraries

In an aspect, a disclosed kit can comprise one or more disclosed components, ingredients, instruments, and/or devices for use in the experiments described in, for instance, Example 1, Example 2, Example 3, Example 4, Example 5, Example 6, Example 7, Example 8, Example 9, Example 10, Example 11, or any combination thereof. In an aspect, a disclosed kit can comprise one or more disclosed components, ingredients, instruments, and/or devices for use to perform one or more time in the experiments described in, for instance, Example 1, Example 2, Example 3, Example 4, Example 5, Example 6, Example 7, Example 8, Example 9, Example 10, Example 11, or any combination thereof.

In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed polymerase, a disclosed buffer, a disclosed ingredient, a disclosed host cell, a disclose plasmid, a disclosed component, a disclosed primer, or any combination thereof, and a label or package insert with instructions for use. In an aspect, suitable containers include, for example, bottles, vials, syringes, blister pack, etc. The containers can be formed from a variety of materials such as glass or plastic. The container can hold a disclosed polymerase, a disclosed buffer, a disclosed ingredient, a disclosed host cell, a disclose plasmid, a disclosed component, a disclosed primer, or any combination thereof, and can have a sterile access port (for example the container may be an intravenous solution bag or a vial having a stopper pierceable by a hypodermic injection needle). The label or package insert can indicate that a disclosed polymerase, a disclosed buffer, a disclosed ingredient, a disclosed host cell, a disclose plasmid, a disclosed component, a disclosed primer, or any combination thereof can be used for treating, preventing, inhibiting, and/or ameliorating a disease or disorder or complications and/or symptoms associated with a disease or disorder (such as, for example, a disease or disorder that can be treated, prevented, and/or managed by one or more disclosed evolved biomolecules). A kit can comprise additional components necessary for administration such as, for example, other buffers, diluents, filters, needles, and syringes.

In an aspect, a disclosed kit can directly evolve one or more biomolecules of interest. In an aspect, a disclosed kit can need less than 5 hours of “hands-on” experiment time to evolve one or more biomolecules of interest. In an aspect, a disclosed kit can directly evolve one or more biomolecules saving about 70% of “hands-on” experiment time when compared to a traditional direct evolution method. In an aspect, a disclosed kit can be used in a method of directing evolution of one or more biomolecules, wherein the method can need less than 5 hours of “hands-on” experiment time to evolve one or more biomolecules of interest. In an aspect, a disclosed kit can be used in a method of directing evolution of one or more biomolecules, wherein the method can save about 70% of “hands-on” experiment time when compared to a traditional directed evolution method.

In an aspect, a disclosed encoded transgene can comprise any gene with a gene product that is directly or indirectly linked to one or more genetic diseases. Such genes include but are not limited to the following: dystrophin including mini- and micro-dystrophins (DMD); titin (TTN); titin cap (TCAP) α-sarcoglycan (SGCA), β-sarcoglycan (SGCB), γ-sarcoglycan (SGCG) or δ-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin α-7 (IGTA7); integrin α-9 (IGTA9); glucosamine(N-acetyl)-6-sulfatase (GNS); galactosamine(N-acetyl)-6-sulfatase (GALNS); β-galactosidase (GLB1); β-glucuronidase (GUSB); hyaluronoglucosaminidase 1 (HYAL1); acid ceramidase (ASAHI); galactosylceramidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase α (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase α class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel β subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); carnitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic β1 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin α (DTNA); protein kinase AMP-activated, noncatalytic T2 (PRKAG2); cysteine- and glycine-rich protein 3 (CSRP3); vinculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cb1D type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histidine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor 33 (TGFβ3); transforming growth factor β receptor, type I (TGFβR1); transforming growth factor β receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2).

In an aspect, a disclosed kit can directly evolve one or more biomolecules of interest that can be used in or relevant to the agricultural arts. In an aspect, a disclosed kit can be used to improve a property and/or characteristic and/or aspect of a crop. As known to those skilled in the art, a crop can be a food crop, a feed crop, a fiber crop, and oil crop, an ornamental crop, and/or an industrial crop. For example, in an aspect of a disclosed kit, a disclosed biomolecule of interest can improve disease resistance and/or can increase or enhance immunity to pests and/or pathogens (including for example, fungi, fungal-like organisms, bacteria, mollicutes, phytoplasmas, viruses, viroids, nematodes, parasitic higher plants, or any combination thereof) in a crop of interest or a crop having commercial, economic, and/or social value. In an aspect of a disclosed kit, a disclosed biomolecule of interest can improve yield of a crop of interest crop of interest or a crop having commercial, economic, and/or social value. In an aspect of a disclosed kit, a disclosed biomolecule of interest can increase or enhance tolerance to abiotic stress in a crop of interest crop of interest or a crop having commercial, economic, and/or social value. In an aspect of a disclosed kit, a disclosed biomolecule of interest can increase or enhance tolerance to drought of a crop of interest crop of interest or a crop having commercial, economic, and/or social value. In an aspect of a disclosed kit, a disclosed biomolecule of interest can increase or enhance resistance to one or more herbicides in a crop of interest or a crop having commercial, economic, and/or social value. In an aspect of a disclosed kit, a disclosed biomolecule of interest can increase or enhance photosynthesis or the rate of photosynthesis in a crop of interest or a crop having commercial, economic, and/or social value. In an aspect of a disclosed kit, a disclosed biomolecule of interest can improve the biomass of a crop of interest or a crop having commercial, economic, and/or social value. In an aspect of a disclosed kit, a disclosed biomolecule of interest can increase and/or enhance the nutritional content of a crop of interest or a crop having commercial, economic, and/or social value. In an aspect of a disclosed kit, a disclosed biomolecule of interest can improve the tolerance to temperature, ultraviolet radiation, or other abiotic factors of a crop of interest or a crop having commercial, economic, and/or social value. In an aspect of a disclosed kit, a disclosed biomolecule of interest can be a fungus (e.g., an entomopathogenic fungi) that can be evolved as a pesticide. In an aspect of a disclosed kit, the directed evolution of one or more biomolecule of interest that improve resistance to pests and/or pathogens can be genetically engineered into a crop of interest or a crop having commercial, economic, and/or social value. In an aspect of a disclosed kit, the directed evolution of one or more biomolecule of interest can be a pheromone of a pest and/or a pathogen that infests and/or damages a crop of interest or a crop having commercial, economic, and/or social value. In an aspect of a disclosed kit, an evolved pheromone can be applied and/or contacted with a crop of interest or a crop having commercial, economic, and/or social value. In an aspect, a disclosed crop of interest or a disclosed crop having commercial, economic, and/or social value can be any crop including, but not limited to rice, maize, wheat, soybean, potatoes, tomatoes, sugarcane, grapes, cotton, apples, onions, sorghums, cucumbers, garlic, bananas, watermelons, mangos, guavas, rapeseed, chilis, peppers, olives, eggplants, oranges, sweet potatoes, barleys, sunflowers, tangerines, mandarins, clementine, yams, cabbages, spinaches, strawberries, peaches, nectarines, tobaccos, coffees, teas, lettuces, peas, blueberries, pumpkins, cassavas, and combinations thereof.

### 3. Plasmids and Cells

Disclosed herein is a plasmid comprising one or more disclosed polynucleotides encoding one or more biomolecules of interest. Disclosed herein is a plasmid comprising one or more disclosed polynucleotides encoding one or more evolved biomolecules of interest. Plasmids and using plasmids are known to the art.

Disclosed herein are cells comprising one or more disclosed polynucleotides encoding one or more biomolecules of interest. Disclosed herein are cells comprising one or more disclosed polynucleotides encoding one or more evolved biomolecules of interest. Disclosed herein are bacterial cells comprising linear plasmid DNA generated during a step of a disclosed method. Disclosed herein are mammalian cells comprising circular plasmid DNA generated during a step of a disclosed method.

### B. Methods of Directed Evolution

Disclosed herein is method of directed evolution of at least one biomolecule of interest to be evolved, the method comprising preparing a circular plasmid DNA comprising at least one promoter for transcription and translation, and a polynucleotide sequence encoding the at least one biomolecule of interest to be evolved; amplifying the entire circular plasmid DNA into an entire linear plasmid DNA by performing a single error prone PCR in the presence of a DNA polymerase fused to a DNA binding protein, wherein the DNA polymerase does not comprise any editing activity; evolving the at least one biomolecule of interest by introducing mutations into the polynucleotide sequence encoding the at least one biomolecule of interest during the single error prone PCR by addition of increased concentrations of magnesium, manganese, and deoxynucleotide triphosphates during the PCR amplification of the entire circular plasmid DNA into linear plasmid DNA containing mutations throughout the DNA sequence; circularizing the linear plasmid DNA into a circular plasmid DNA in bacteria; incorporating the circular plasmid DNA into a host cell; assessing at least one evolved biomolecule for one or more resultant activities of interest in the host cell.

Disclosed herein is method of directed evolution of at least one biomolecule of interest to be evolved, the method comprising preparing a circular plasmid DNA comprising at least one promoter for transcription and translation, and a polynucleotide sequence encoding the at least one biomolecule of interest to be evolved; amplifying the entire circular plasmid DNA into an entire linear plasmid DNA by performing a single error prone PCR in the presence of a DNA polymerase fused to a DNA binding protein, wherein the DNA polymerase does not comprise any editing activity; evolving the at least one biomolecule of interest by introducing mutations into the polynucleotide sequence encoding the at least one biomolecule of interest during the single error prone PCR by addition of increased concentrations of magnesium, manganese, and deoxynucleotide triphosphates during the PCR amplification of the entire circular plasmid DNA into linear plasmid DNA containing mutations throughout the DNA sequence; circularizing the linear plasmid DNA into a circular plasmid DNA in bacteria; incorporating the circular plasmid DNA into a host cell; assessing at least one evolved biomolecule for one or more resultant activities of interest in the host cell, wherein the method does not comprise use of a virus or viral vector.

In an aspect, a disclosed method can comprise a single PCR mammalian expression generated array for the directed evolution of biomolecules. In an aspect, a disclosed method can comprise a single PCR mammalian expression generated array for the directed evolution of biomolecules, wherein the use of viral vectors or viruses is avoided.

In an aspect of a disclosed method, the mutation efficiency can be at least about 1-in-400 base pairs, about 1-in-350 base pair, 1-in-300 base pairs, 1-in-250 base pairs, 1-in-200 base pairs, 1-in-150 base pairs, 1-in-100 base pairs. In an aspect of a disclosed method, the mutation efficiency can be about 1-in-200 base pairs.

In an aspect of a disclosed method, a disclosed DNA polymerase fused to a DNA binding protein can amplify the plasmid in its entirety. In an aspect, a disclosed entire plasmid can be amplified by the DNA polymerase fused to a DNA binding protein. In an aspect, a disclosed DNA polymerase fused to a DNA binding protein can amplify at least about 0.1 kilobases of DNA. In an aspect, a disclosed DNA polymerase fused to a DNA binding protein can amplify about 0.1 kilobases of DNA to about 15 kilobases of DNA. In an aspect, a disclosed DNA polymerase fused to a DNA binding protein can amplify more than about 15 kilobases of DNA. In an aspect, a disclosed DNA polymerase fused to a DNA binding protein can amplify more than about 15 kilobases of DNA after optimization of at least one aspect of the system wherein the at least one aspect of the system comprises reaction temperature, extension time, number of cycles, amount of DNA primers, or any combination thereof

In an aspect, a disclosed method can further comprise sequencing of the polynucleotide.

In an aspect, a disclosed method can further comprise sequencing of the at least one evolved biomolecule to identify one or more mutations. In an aspect, a disclosed method can further comprise subjecting the at least one evolved biomolecule having at least one or more resultant activity or activities of interest attributed to the at least one or more mutations identified by sequencing, one or more times.

In an aspect, a disclosed method can further comprise repeating one or more steps of the disclosed method of directed evolution. In an aspect, a disclosed method can further comprise repeating one or more steps of the disclosed method of directed evolution about two or more times.

In an aspect of a disclosed method, amplifying the entire circular plasmid DNA into an entire linear plasmid DNA by performing a single error prone PCR in the presence of the DNA polymerase fused to a DNA binding protein can generate a DNA library encoding at least about 100 evolved biomolecules.

In an aspect of a disclosed method, amplifying the entire circular plasmid DNA into an entire linear plasmid DNA by performing a single error prone PCR in the presence of the DNA polymerase fused to a DNA binding protein can generate a DNA library encoding about one million to about one trillion evolved biomolecules.

In an aspect, a disclosed method can be used to metabolically engineer, to create one or more synthetic organisms for production of unnatural amino acids for creation of one or more proteins with new functions, to produce one or more biological circuits for molecule sensing and memory storage, to genetically reprogram, or any combination thereof.

In an aspect of a disclosed method, translating the plasmid into linear plasmid DNA by performing PCR in the presence of the DNA polymerase fused to a DNA binding protein can generate a biomolecule DNA library. In an aspect of a disclosed method, translating the plasmid into linear plasmid DNA by performing PCR in the presence of the DNA polymerase fused to a DNA binding protein can generate an antibody DNA library.

In an aspect of a disclosed method, translating the plasmid into linear plasmid DNA by performing PCR in the presence of the DNA polymerase fused to a DNA binding protein can generate a gRNA DNA library, a CRISPR DNA library, a Cas DNA library, a Zinc Finger DNA library, a TALEN DNA library, a restriction enzyme DNA, or any combination thereof.

In an aspect of a disclosed method, a disclosed gRNA DNA library, the CRISPR DNA library, the Cas DNA library, the Zinc Finger DNA library, the TALEN DNA library, the restriction enzyme DNA, or any combination thereof can be transcribed in vitro or inside the host cell. In an aspect, a disclosed gRNA DNA library can be used in combination with CRISPR/Cas proteins to site-specifically cut genomic DNA to knock out, to change nucleotides, to change amino acids upon mRNA translation, to knock in added DNA to screen for cellular phenotypes, or any combination. CRISPR and Cas endonuclease are discussed supra. In an aspect, a disclosed CRISPR DNA library, the Cas DNA library, the Zinc Finger DNA library, the TALEN DNA library, the restriction enzyme DNA, or any combination thereof can be transcribed and translated into at least one protein. In an aspect, a disclosed CRISPR DNA library, the Cas DNA library, the Zinc Finger DNA library, the TALEN DNA library, the restriction enzyme DNA, or any combination thereof can be translated into at least one protein that is evolved to reduce off-target cutting and enhance specificity, to create new PAM sequence recognition, to produce improved dsDNA and/or nickase breakage with enhanced kinetics to speed the reaction, to create new variants for producing knock out, to create nucleotide changes, to knock in with added DNA, to introduce site-specific modification of genomic or recombinant DNA, or any combination thereof.

In an aspect, a disclosed method of directed evolution, a disclosed plasmid can comprise two biomolecules of interest to be evolved. In an aspect, a disclosed plasmid can comprise at least two or more biomolecules of interest to be evolved, wherein each biomolecule of interest comprises a nucleic acid, an oligonucleotide, an oligonucleoside, a protein, a peptide, an enzyme, an amino acid, a lipid, an organic molecule, a small molecule, or any combination thereof.

In an aspect, a disclosed molecule of interest can comprise a silencing RNA or a guide RNA. In an aspect, a disclosed silencing RNA can be expressed to knock down gene expression and/or increase the expression of a protein library to identify one or more phenotypes.

In an aspect, a disclosed silencing RNA can be expressed to knock down gene expression and/or increase the expression of a protein of interest. In an aspect, a disclosed silencing RNA can be expressed to knock down gene expression and/or increase the expression of a protein implicated in a disease or disorder. In an aspect, a disclosed guide RNA can be expressed for genomic DNA editing in a library of CRISPR/CasX mutants.

In an aspect, a disclosed RNA aptamer can be selected for binding to a protein expressed on the same plasmid. In an aspect, a disclosed protein can comprise a protein endogenous to a host cell, a protein exogenous to a host cell, or any combination thereof. In an aspect, a disclosed protein endogenous to a host cell, a protein exogenous to a host cell, or any combination thereof can increase the number of small molecules including inorganic salts in a cell, excreted by a cell, outside of a cell, or any combination thereof.

In an aspect, a disclosed small molecule can comprise one or more inorganic salts. In an aspect, a disclosed organic molecule can comprise organic molecules native to a host cell, organic molecules not native to a host cell, or any combination thereof. In an aspect, a disclosed organic molecule can comprise am intracellular organic molecule, an extracellular organic molecule, or any combination thereof. In an aspect, a disclosed organic molecule can comprise an amino acid. Amino acids are discussed supra.

In an aspect, a disclosed biomolecule of interest can comprise an antisense oligonucleotide (ASO), a small interfering RNA (siRNA), a silencing RNA, a DNA aptamer, RNA aptamer, a transfer RNA (tRNA), a ribosomal RNA (rRNA), a long non-coding RNA (lncRNA), a microRNAs (miRNAs), a small nucleolar RNA (snoRNA), a small nuclear RNA (snRNAs), a piwi-interacting RNA (piRNA), a messenger RNA (mRNA), a gene-editing guide RNA (gRNA), or any combination thereof.

In an aspect, a disclosed biomolecule of interest can comprise a full-length antibody, an antigen binding fragment of a full-length antibody, a Fab fragment, a single chain antibody (scFv), a diabody, a triabody, a minibody, a nanobody, a single-domain antibody, camelids, or any combination thereof.

In an aspect, a disclosed biomolecule of interest can comprise an enzyme that can perform novel protein, RNA, and/or DNA modifications, or can perform a combination of novel protein, RNA, and/or DNA modifications.

In an aspect of a disclosed method, a disclosed biomolecule of interest can comprise an enzyme that can elevate native small molecules. In an aspect, a disclosed biomolecule of interest can comprise an enzyme that can create novel small molecules not typically or not normally present inside a specific cell (such as, for example, a mammalian cell or a bacterial cell). In an aspect, a disclosed biomolecule of interest can comprise an enzyme that can degrade plastic or waste products, thereby forming useful molecules. In an aspect, a disclosed biomolecule of interest comprises an enzyme degrade carbon dioxide or other gases to create synthetically useful molecules and reduce greenhouse gases. In an aspect, a disclosed biomolecule of interest can comprise an enzyme that can degrade plastic or waste products, thereby forming useful molecules, can degrade carbon dioxide or other gases to create synthetically useful molecules and reduce greenhouse gases, or any combination thereof. In an aspect, a disclosed method can be used in metabolomic engineering. In an aspect, a disclosed method can be used to create one or more synthetic organisms for production of unnatural amino acids, wherein one or more unnatural amino acids can be used for creation of one or more proteins with new functions. In an aspect, a disclosed method can be used to produce one or more biological circuits for molecule sensing and memory storage, genetic reprogramming, or any combination thereof.

In an aspect, a disclosed method can directly evolve one or more biomolecules of interest that can be used in or relevant to the agricultural arts. In an aspect, a disclosed method can be used to improve a property and/or characteristic and/or aspect of a crop. As known to those skilled in the art, a crop can be a food crop, a feed crop, a fiber crop, and oil crop, an ornamental crop, and/or an industrial crop. For example, in an aspect of a disclosed method, a disclosed biomolecule of interest can improve disease resistance and/or can increase or enhance immunity to pests and/or pathogens (including for example, fungi, fungal-like organisms, bacteria, mollicutes, phytoplasmas, viruses, viroids, nematodes, parasitic higher plants, or any combination thereof) in a crop of interest or a crop having commercial, economic, and/or social value. In an aspect of a disclosed method, a disclosed biomolecule of interest can improve yield of a crop of interest crop of interest or a crop having commercial, economic, and/or social value. In an aspect of a disclosed method, a disclosed biomolecule of interest can increase or enhance tolerance to abiotic stress in a crop of interest crop of interest or a crop having commercial, economic, and/or social value. In an aspect of a disclosed method, a disclosed biomolecule of interest can increase or enhance tolerance to drought of a crop of interest crop of interest or a crop having commercial, economic, and/or social value. In an aspect of a disclosed method, a disclosed biomolecule of interest can increase or enhance resistance to one or more herbicides in a crop of interest or a crop having commercial, economic, and/or social value. In an aspect of a disclosed method, a disclosed biomolecule of interest can increase or enhance photosynthesis or the rate of photosynthesis in a crop of interest or a crop having commercial, economic, and/or social value. In an aspect of a disclosed method, a disclosed biomolecule of interest can improve the biomass of a crop of interest or a crop having commercial, economic, and/or social value. In an aspect of a disclosed method, a disclosed biomolecule of interest can increase and/or enhance the nutritional content of a crop of interest or a crop having commercial, economic, and/or social value. In an aspect of a disclosed method, a disclosed biomolecule of interest can improve the tolerance to temperature, ultraviolet radiation, or other abiotic factors of a crop of interest or a crop having commercial, economic, and/or social value. In an aspect of a disclosed method, a disclosed biomolecule of interest can be a fungus (e.g., an entomopathogenic fungi) that can be evolved as a pesticide. In an aspect of a disclosed method, the directed evolution of one or more biomolecule of interest that improve resistance to pests and/or pathogens can be genetically engineered into a crop of interest or a crop having commercial, economic, and/or social value. In an aspect of a disclosed method, the directed evolution of one or more biomolecule of interest can be a pheromone of a pest and/or a pathogen that infests and/or damages a crop of interest or a crop having commercial, economic, and/or social value. In an aspect of a disclosed method, an evolved pheromone can be applied and/or contacted with a crop of interest or a crop having commercial, economic, and/or social value. In an aspect, a disclosed crop of interest or a disclosed crop having commercial, economic, and/or social value can be any crop including, but not limited to rice, maize, wheat, soybean, potatoes, tomatoes, sugarcane, grapes, cotton, apples, onions, sorghums, cucumbers, garlic, bananas, watermelons, mangos, guavas, rapeseed, chilis, peppers, olives, eggplants, oranges, sweet potatoes, barleys, sunflowers, tangerines, mandarins, clementine, yams, cabbages, spinaches, strawberries, peaches, nectarines, tobaccos, coffees, teas, lettuces, peas, blueberries, pumpkins, cassavas, and combinations thereof.

In an aspect of a disclosed method, a disclosed plasmid can be any plasmid having one or more protomers for transcription and translation. In an aspect, a disclosed plasmid can be any plasmid known to the art. In an aspect, a disclosed plasmid can be a bacterial expression plasmid, a mammalian expression plasmid, an insect expression plasmid, a viral expression plasmid, a species-specific host organism expression plasmid, or any combination thereof.

In an aspect of a disclosed method, the mutation efficiency can be at least about 1-in-400 base pairs, about 1-in-350 base pair, 1-in-300 base pairs, 1-in-250 base pairs, 1-in-200 base pairs, 1-in-150 base pairs, 1-in-100 base pairs. In an aspect of a disclosed method, the mutation efficiency can be about 1-in-200 base pairs.

In an aspect of a disclosed method, a host cell can comprise the plasmid comprising the one or more promoters for transcription and translation. In an aspect, a disclosed host cell can comprise a bacterial cell, a yeast cell, an insect cell, a mammalian cell, a species-specific host cell, or any combination thereof. In an aspect, a disclosed host cell can be a human cell.

In an aspect, a disclosed method can evolve at least one biomolecule of interest by introducing one or more mutations into the polynucleotide sequence encoding at least one biomolecule of interest, and circularizing the linear plasmid DNA by electroporating the linear plasmid DNA in bacteria. In an aspect, a disclosed circular plasmid DNA comprises a bacterial library.

In an aspect of a disclosed method, a biomolecule of interest to be evolved can comprise a silencing RNA or a guide RNA. In an aspect of a disclosed method, a disclosed silencing RNA can be expressed to knock down gene expression and/or increase the expression of a protein library to identify one or more phenotypes. In an aspect of a disclosed method, a disclosed silencing RNA can be expressed to knock down gene expression and/or increase the expression of a protein of interest. For example, in an aspect of a disclosed method, a disclosed silencing RNA can be expressed to knock down gene expression and/or increase the expression of a protein implicated in a disease or disorder. Genes that are implicated in a disease or disorder are known to the art and can include ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1 L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANE1, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6 L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREB1 L, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12 L, MED13, MED13 L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1 L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAI1, RALGAPA1, RANBP2, RB1CC1, RELN, RERE, REV3 L, RIC1, RIMS1, RIMS2, RNF213, ROBO1, ROBO2, ROBO3, ROS1, RP1, RP1 L1, RTTN, RUSC2, RYR1, RYR2, SACS, SAMD9, SAMD9 L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1 L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect of a disclosed method, a disclosed silencing RNA can be expressed to knock down gene expression and/or increase the expression of a protein implicated in a disease or disorder with a gene product that is directly or indirectly linked to one or more genetic diseases. Such genes include but are not limited to the following: dystrophin including mini- and micro-dystrophins (DMD); titin (TTN); titin cap (TCAP) α-sarcoglycan (SGCA), 0-sarcoglycan (SGCB), γ-sarcoglycan (SGCG) or δ-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); β-galactosidase (GLB1); β-glucuronidase (GUSB); hyaluronoglucosaminidase 1 (HYAL1); acid ceramidase (ASAHI); galactosylceramidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase α (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase α class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel β subunit 4 (SCN4B); sodium voltage-gated channel α subunit 5 (SCN5A); sodium voltage-gated channel a subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); carnitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic β1 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin α (DTNA); protein kinase AMP-activated, noncatalytic γ2 (PRKAG2); cysteine- and glycine-rich protein 3 (CSRP3); vinculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cb1D type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histidine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor 33 (TGFβ3); transforming growth factor β receptor, type I (TGFβR1); transforming growth factor β receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2).

In an aspect, a disclosed in vitro or cell lysate can be used to produce the biomolecules.

In an aspect, a disclosed method can comprise assessing the one or more evolved biomolecules for one or more resultant activity or resultant activities. In an aspect, assessing the one or more evolved biomolecules for one or more resultant activity or resultant activities can comprise using FACS, gel electrophoresis, ELISA, high-throughput plate reader fluorescence or absorbance assays, high-content imaging of single cells, mass spectrometry proteomics, metabolomics, kinemics, omics analysis, Western blot, Northern blot, Southern blot, fluorescence in situ hybridization (FISH), spatial transcriptomics, fluorescence microscopy, fixed expansion microscopy, electron microscopy, reverse transcription and PCR amplification, Sanger sequencing, Next Generation Sequencing (NGS), or any combination thereof.

In an aspect, a disclosed plasmid comprising at least one promoter for transcription and translation and the polynucleotide sequence encoding the at least one biomolecule of interest to be evolved, can further comprise a polynucleotide sequence encoding for at least one sensor. In an aspect, a disclosed sensor can comprise a fluorescent protein, a small molecule binding protein sensor, a GPCR-based sensor, an enzyme activity sensor, a protein modification sensor, an RNA modification sensor, a DNA modification sensor, a biomolecule modification sensor, a protein-protein interaction (PPi) sensor, a fluorescent ubiquitination-based cell cycle indicator (FUCCI), a cell membrane voltage sensor, or any combination thereof.

In an aspect, a disclosed small molecule binding protein sensor can detect calcium, magnesium, zinc, a small inorganic molecule, glucose, lactose, cyclic adenosine monophosphate, dopamine, acetylcholine, a small molecule sugar, a neurotransmitter, a molecule used inside cells, or any combination thereof. In an aspect, a disclosed GPCR-based sensor can detect calcium, magnesium, zinc, a small inorganic molecule, glucose, lactose, cyclic adenosine monophosphate, dopamine, acetylcholine, a small molecule sugar, a neurotransmitter, a molecule used inside cells, or any combination thereof.

In an aspect of a disclosed plasmid comprising at least one promoter for transcription and translation can comprise a polynucleotide sequence encoding at least one fluorescent protein of interest to be evolved. In an aspect, a disclosed fluorescent protein of interest is evolved to improve brightness, enhance biophysical properties, bind new chromophore molecules, create reversible photo-activatable fluorescent proteins, create irreversible photo-activatable fluorescent proteins, or any combination thereof.

In an aspect, a disclosed plasmid comprising at least one promoter for transcription and translation can comprise a polynucleotide sequence encoding an optogenetic element of interest to be evolved. In an aspect, a disclosed optogenetic element can comprise a channel rhodopsin, a halorhodopsin, a light-oxygen voltage (LOV) domain, a phytochrome light-activated enzyme, a biomolecule activated by light, a biomolecule activated or deactivated by light, or any combination thereof. In an aspect, a disclosed biomolecule of interest can be evolved to alter kinetics, light modulation and/or enhance performance.

In an aspect, a disclosed biomolecule of interest to be evolved can be used as one or more research tools. In an aspect, a disclosed research tool can comprise a research tool to modulate protein expression within a cell or a model organism, to perform gene editing within a cell or a model organism, to modulate small molecule concentrations within a cell or a model organism, to modulate enzyme activity within a cell or a model organism, to bind small molecules to visualize location, to identify a protein by specific epitope binding, or any combination thereof.

In an aspect, a disclosed model organism can comprise a vertebrate, an invertebrate, or any combination thereof.

In an aspect, a disclosed model organism can comprise bacteria, yeast, fish, worms, insects, rodents, or any combination thereof. In an aspect of a disclosed method, a disclosed biomolecule of interest to be evolved can be used as a prophylactic for a disease or a disorder.

In an aspect of a disclosed method, a disclosed biomolecule of interest to be evolved can be used to treat and/or prevent and/or manage a disease or a disorder.

In an aspect, a disclosed evolved biomolecule can be used to modulate protein number, create chaperone proteins for elevated proteins at proper cellular location, create tRNAs that suppress nonsense and frameshift mutations to produce full-length genes, produce non-coding RNAs to alter and engineer cellular functions, identify unknown functions of RNA, create immunotherapies, create small molecules, create therapeutic proteins, or any combination thereof.

In an aspect, disclosed disease or the disorder can be cancer. In an aspect, a subject having or suspected of having a disease or a disorder can be a human patient. In an aspect, a human patient can have been diagnosed as having or can be suspected of having cancer. Cancer is discussed supra and is known to the art. In an aspect, a disclosed disease or the disorder can be a vision disorder, a sleep disorder, a circadian disorder, or any combination thereof. In an aspect, a disclosed evolved biomolecule of interest can be used as a treatment or prophylactic to be administered to a subject having or suspected of having a disease or a disorder. In an aspect, a disclosed human patient can be diagnosed as having or can be suspected of having a vision disorder, a sleep disorder, a circadian disorder, or any combination thereof.

In an aspect, a disclosed method of directed evolution can comprise repeating one or more disclosed steps. In an aspect, a disclosed method of directed evolution can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount or the concentration of one or more of the disclosed ingredients, components, and/or agents used in the method, or by changing the frequency of use of one or more of the disclosed ingredients, components, and/or agents used in the method, or by changing the duration of time the one or more of the disclosed ingredients, components, and or agents are used in the method.

Disclosed herein is a method for directed evolution, the method comprising preparing DNA library by performing error prone PCR mutagenesis; circularizing plasmid DNA by electroporating linear plasmid DNA in bacteria; screening bacteria and purifying circular plasmid DNA; growing mammalian cells; lipofecting mammalian cells with circular plasmid DNA; subjecting the mammalian cells to cell sorting; lysing the mammalian cells and recovering circular plasmid DNA; electroporating bacteria and plating electroporated bacteria; lipofecting circular plasmid DNA into mammalian cells and screening lipofected mammalian cells; lipofecting circular DNA into mammalian cells and screening lipofected mammalian cells; and identifying amino acid mutations by sequencing and analyzing DNA.

In an aspect, a disclosed method can evolve one or more biomolecules of interest using less than 5 hours of hands-on experiment time or tech time. In an aspect, a disclosed method can evolve one or more biomolecules of interest using less than 70% of the hands-on experiment or tech time when compared to a traditional method of directed evolution.

In an aspect, a disclosed method of directed evolution can comprise preparing DNA library by performing error prone PCR mutagenesis. In an aspect, a disclosed method of directing evolution of one or more biomolecules can comprise preparing a DNA library by performing error prone PCR mutagenesis. In an aspect, a disclosed method of directing evolution of one or more biomolecules can comprise performing error prone PCR mutagenesis to create a DNA library. In an aspect of a disclosed method of directing evolution of one or more biomolecules, wherein error prone PCR mutagenesis is performed to create a DNA library. In an aspect of a disclosed method of directing evolution of one or more biomolecules, wherein a DNA library is created through error prone PCR mutagenesis. In an aspect, error prone PCR can introduce random mutants into any DNA sequence encoding one or more biomolecules are interest.

In an aspect, a disclosed error prone PCR can comprise two or more primers. In an aspect, disclosed primers can be designed to amplify a disclosed plasmid DNA. In an aspect, disclosed primers can be designed to amplify the entirety of a disclosed plasmid DNA. In an aspect, a disclosed DNA plasmid can be commercially purchases or synthesized de novo.

In an aspect, a disclosed plasmid can be a pDuEx2 vector that allows for expression in bacterial cells and mammalian cells. A pDuEX vector can comprise a pcDNA backbone. In an aspect, a disclosed DNA plasmid can be a circular DNA plasmid.

In an aspect, a disclosed DNA plasmid can be any plasmid that can be amplified by a disclosed DNA polymerase that lacks editing activity and/or capability. For example, a disclosed plasmid can be amplified with Platinum II Hot-Start DNA Polymerase. In an aspect, a disclosed plasmid can comprise a plasmid for bacterial expression, for mammalian expression, for insect expression, for viral expression, for species-specific expression, or for any combination thereof.

In an aspect, a disclosed plasmid can comprise a polynucleotide encoding two or more biomolecules. In an aspect, a disclosed method can comprise multiple DNA plasmids.

In an aspect, a disclosed circular DNA plasmid can comprise a polynucleotide sequence encoding a disclosed biomolecule of interest. In an aspect, a disclosed circular DNA plasmid can comprise a polynucleotide sequence encoding one or more disclosed biomolecules of interest. In an aspect, a disclosed circular DNA plasmid can comprise a polynucleotide sequence encoding a disclosed biomolecule of interest to be evolved in a disclosed method. In an aspect, a disclosed circular DNA plasmid can comprise a polynucleotide sequence encoding one or more disclosed biomolecules of interest to be evolved in a disclosed method.

In an aspect, a disclosed circular DNA plasmid can comprise a polynucleotide sequence encoding a disclosed nucleic acid, oligonucleotide, oligonucleoside, protein, peptide, enzyme, amino acid, lipid, organic molecule, small molecule, or any combination thereof to be evolved in a disclosed method.

In an aspect, a disclosed circular DNA plasmid can comprise a polynucleotide sequence encoding one or more disclosed nucleic acids, oligonucleotides, oligonucleosides, proteins, peptides, enzymes, amino acids, lipids, organic molecules, small molecules, or any combination thereof to be evolved in a disclosed method.

In an aspect, a disclosed forward primer can comprise the sequence of SEQ ID NO: 01. In an aspect, a disclosed reverse primer can comprise the sequence of SEQ ID NO: 02. In an aspect, a disclosed forward primer can be directed to a sequence before a disclosed promoter. In an aspect, a disclosed primer set can be changeable to amplify any disclosed plasmid. Primer design is known to the skilled person in the art. In an aspect, a disclosed DNA polymerase can be polymerase that lacks editing activity and/or capability.

In an aspect, a disclosed DNA polymerase can be fused to one or more DNA-binding proteins. As known to the art, DNA-binding proteins can perform important functions in a great number of biological activities. In an aspect, DNA-binding proteins can interact with ssDNA (single-stranded DNA) or dsDNA (double-stranded DNA), and DNA-binding proteins can be categorized as single-stranded DNA-binding proteins (SSBs) and double-stranded DNA-binding proteins (DSBs).

In an aspect, a disclosed DNA polymerase that lacks editing activity and/or capability can be fused to a disclosed double-stranded DNA binding protein (dsDNA bp or dsDNA binding protein). Double-stranded DNA binding proteins are known to the art and can be located using a UniProt search. In an aspect, a disclosed DNA polymerase that lacks editing activity and/or capability fused to a disclosed dsDNA binding protein can increase processivity and/or can allow for amplification of >7 kb. In an aspect, a disclosed DNA polymerase that lacks editing activity and/or capability fused to a disclosed dsDNA binding protein can lack the editing ability to introduce mutations with error prone PCR.

In an aspect, a disclosed first polynucleotide encodes small ultra-red fluorescent protein (smURFP) and a disclosed second polynucleotide encodes Synechocystis heme oxygenase (HO-1). In an aspect, a disclosed second polynucleotide can only be expressed in E. coli and can produce biliverdin for covalent attachment to smURFP for fluorescence. In an aspect, biliverdin and biliverdin dimethyl ester are not E. coli membrane permeant and cell lysis allows for chemical addition.

In an aspect, a disclosed first polynucleotide can be expressed in both mammalian and bacterial cells while a disclosed second polynucleotide can be expressed only in E. coli. In an aspect, a disclosed DNA plasmid can comprise an internal ribosomal entry site (IRES). In an aspect, a disclosed DNA plasmid can comprise a self-cleaving peptide (P2A, E2A, F2A, and T2A) for the expression of two genes in mammalian cells on a bicistronic mRNA.

In an aspect, a disclosed DNA plasmid can comprise one or more promoters. In an aspect, a disclosed promoter can be selected to drive high, medium, or low expression of one or more disclosed polynucleotides. Promoters are discussed supra. In an aspect, a disclosed promoter can be a cell-specific promoter. In an aspect, a disclosed cell-specific promoter can allow the expression of genes only in a specific cell type. Inducible promoters can allow for temporal control of an encoded biomolecule of interest.

In an aspect, the amplification of a disclosed plasmid can be unique for the evolution of one or more disclosed biomolecules.

In an aspect, a disclosed method can employ about 10 ng-80 ng of circular plasmid DNA. In an aspect, a disclosed method can employ about 10 ng, about 20 ng, about 30 ng, about 40 ng, about 50 ng, about 60 ng, about 70 ng, and about 80 ng of circular plasmid DNA. In an aspect, a disclosed method can employ about 60 ng of circular plasmid DNA.

In an aspect, a large amount of circular plasmid DNA can be necessary because the error prone PCR is linear and does not appear to be exponentially amplified. In an aspect, increase the DNA template can significantly increase the DNA product. In an aspect, disclosed primers can be prepared in nuclease-free water. In an aspect, a disclosed circular plasmid DNA can be prepared in nuclease-free water. In an aspect, a MgCl₂ solution can be prepared in nuclease-free water, can be aliquoted, and stored at −20° C.

In an aspect, a dNTP solution can be prepared and/or purchased. In an aspect, a disclosed dNTP solution can be aliquoted and/or stored. In an aspect, a disclosed frozen dNTP solution can be thawed on ice. Care should be taken to avoid multiple free-thaws. Storage can be at −80° C.

In an aspect, a MnCl₂ solution can be prepared in nuclease-free water, can be aliquoted, and stored at −20° C.

In an aspect, the disclosed primers, disclosed DNA, disclosed polymerase, disclosed dNTPs, disclosed MgCl₂ solution, disclosed MnCl₂ solution, or any combination thereof can be stored on ice. In an aspect, a user can thaw disclosed primers, DNA, MgCl₂, and MnCl₂ solutions using fingers and/or water baths. Care should be taken to avoid prolonged heating of disclosed DNA.

In an aspect, the generation of a disclosed DNA library by error prone PCR can comprise the listing of components provided in Table 3. In an aspect, a disclosed error prone PCR can comprise 1 cycle (3 min at 95° C.), 35 cycles (30 sec at 95° C., 30 sec at 63° C., and 1:30 min at 68° C.), 1 cycle (10 min at 68° C.) with a 50 μL reaction volume.

In an aspect, after a disclosed error prone PCR completes, the solution can be briefly vortexed and centrifuged. In an aspect, the Dpn1 restriction enzyme and 10× Tango buffer or similar restriction enzyme from a different supplier can be placed on ice. In an aspect, care should be taken to use a buffer that came with the DpnI restriction enzyme. In an aspect, a disclosed PCR reaction can comprise about 50 μL. In an aspect, a disclosed 50 μL PCR reaction can comprise 3 μL water, 6 μL 10× Tango buffer, and 1 μL DpnI. In an aspect, a disclosed volume can be adjusted to 60 μL. In an aspect, a disclosed buffer volume can be 1/10 the final volume with 1.0 μL of DpnI. In an aspect, a disclosed reaction can be incubated at 37° C. for about 2 hours. In an aspect, a DpnI restriction digest can remove methylated circular plasmid DNA to avoid selecting the disclosed starting DNA.

In an aspect, a disclosed fluorescent protein can be dimmer or brighter, while different wavelengths can be selected by changing excitation and emission filters. In an aspect, removing the disclosed methylated template circular plasmid DNA can reduce the possibility of choosing the same fluorescent protein sequence. In an aspect, a long incubation time can ensure multiple cuts of a disclosed methylated template circular plasmid DNA to avoid recircularization in E. coli.

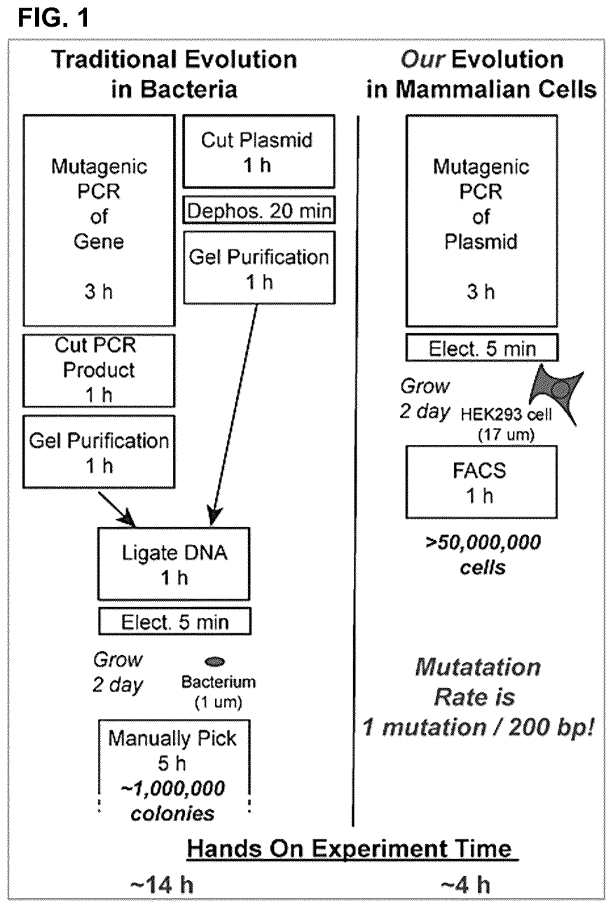

In an aspect, a disclosed PCR product—linear plasmid DNA (FIG. 1)—can be purified using the DNA Clean & Concentrator-5 Kit. In an aspect, a disclosed 2:1 ratio of DNA binding buffer can be added to a disclosed PCR product. In an aspect, a disclosed solution can be transferred to a spin column and can be centrifuged at about 16,000 RCF for about 1 min. In an aspect, flow-through can be discarded. In an aspect, a disclosed column can be washed twice with a wash buffer (e.g., 200 μL for a 400 μL wash buffer total).

In an aspect, a disclosed column can be centrifuged for about 1 min at about 16,000 RCF after each wash. In an aspect, a disclosed flow-through can be discarded. In an aspect, a disclosed column can be centrifuged for about 1 min at about 16,000 RCF to remove the excess buffer. A disclosed spin column can be transferred to a clean Eppendorf tube (e.g., 1.5 mL) and H₂O (e.g., 10 μL of 72° C.) can be applied directly to a disclosed column matrix. In an aspect, a disclosed column matrix can be centrifuged at about 16,000 RCF for about 1 min to elute DNA.

In an aspect, a disclosed purified linear plasmid DNA can be quantified using, for example, the NanoDrop One^{C} UV-Vis Spectrophotometer.

In an aspect, a disclosed yield of linear plasmid DNA after DpnI digestion can be typically ˜20-300 ng/μL. Three replicate reactions can be performed to ensure 100 ng of linear plasmid DNA for the next step.

In an aspect, disclosed purified, the 3′-Adenine is removed from the linear PCR DNA can be prepared (e.g., Linear PCR DNA (˜250 ng/μL) (e.g., 10 μL)), T4 DNA Polymerase Reaction Buffer (e.g., 2.5 μL), 10 mM dNTPs (e.g., 2.5 μL), and H₂O (e.g., 7.25 μL). This can be gently vortexed and centrifuged (e.g., T4 DNA Polymerase (e.g., 0.75 μL) was added). In an aspect, this can be gently vortexed and centrifuged. In an aspect, a disclosed final volume can be about 23 μL and can be incubated at about 12° C. for about 15 minutes.

In an aspect, a disclosed reaction can be stopped with addition of 0.5 M EDTA (e.g., about 0.4 μL) and can be heat inactivated (e.g., 75° C. for about 20 minutes).

In an aspect, a disclosed reaction can be created using about Ligase Buffer (e.g., 4 μL), Linear PCR DNA with 3′-Adenine overhang removed (e.g., 5 μL), and H₂O (e.g., 10 μL). This can be gently vortexed and centrifuged. In an aspect, T4 DNA Ligase (e.g., 1 μL) can be added, gently vortexed and centrifuged for a total final volume of about 20 μL.

In an aspect, this can be incubated at room temperature for about 1 hour or at 14° C. for 12 hours. In an aspect, there can an alternative approach. In an aspect, a disclosed alternative approach after the quantification can comprise the purified, after the Linear PCR DNA and can be as follows: Linear PCR DNA (˜250 ng/μL) (e.g., 10.0 μL), T4 DNA Polymerase Reaction Buffer (e.g., 2.5 μL), 10 mM dNTPs (e.g., 2.5 μL), and H₂O (e.g., 7.25 μL). The solution can be gently vortexed and centrifuged. T4 DNA Polymerase (e.g., 0.75 μL) can be added. The solution can be again gently vortexed and can be centrifuged. The final volume can be 23 μL. The solution can be incubated at 12° C. for 15 minutes. The reaction can be stopped with addition of 0.4 μL 0.5 M EDTA and can be heat inactivated at 75° C. for 20 minutes. In an aspect, the following reaction can be created: Ligase Buffer (e.g., 4 μL), Linear PCR DNA with 3′-Adenine overhang removed (e.g., 5 μL), and of H₂O (e.g., 10 μL). This solution can be gently vortexed and centrifuged. In an aspect, T4 DNA Ligase (e.g., 1 μL) can be added, which can be then gently vortexed and centrifuged. The final volume can be 20 μL. The solution can be incubated at room temperature for 1 hour or 14° C. for 12 hours.

In an aspect, a disclosed method of directing evolution of one or more biomolecules can comprise circularizing plasmid DNA by electroporating linear plasmid DNA into bacteria. In an aspect, a disclosed method of directing evolution of one or more biomolecules can comprise electroporating linear plasmid DNA into E. coli to circularize plasmid DNA.

In an aspect, circularizing a plasmid DNA by electroporating linear plasmid DNA in bacteria can comprise one or more of the following components/ingredients: 100 ng of purified linear plasmid DNA, Super Optimal broth with Catabolite repression (SOC) media is Hanahan's Broth (SOB medium) (Millipore Sigma, H8032) with 20 mM D-(+)-Glucose (Millipore Sigma, G8270), Ampicillin (Millipore Sigma, A1593), One Shot TOP10 Electrocompetent E. coli (Invitrogen, C664-55), Gene Pulser Xcell Total Electroporation system (BioRad, 1652660), LB (Millipore Sigma, L3522) with 100 μg/mL Ampicillin, 0.2 cm electroporation cuvettes (BioRad, 1652086), QIAprep Spin Miniprep Kit (Qiagen, 27104), and NanoDrop One^{C} UV-Vis Spectrophotometer (ThermoFisher, ND-ONEC-W), or any combination thereof. In an aspect, a disclosed ingredient/component/instrument can be substituted for another industry accepted equivalent for that ingredient/component/instrument.

In an aspect, circularizing a plasmid DNA by electroporating linear plasmid DNA in bacteria or E. coli can comprise mixing the three disclosed reactions to obtain 100 ng of linear plasmid DNA samples together. In an aspect, the concentration of a disclosed linear plasmid DNA sample can be determined with the NanoDrop One^{C} UV-Vis Spectrophotometer (or an equivalent spectrophotometer).

In an aspect, the volume needed for 100 ng of DNA can be calculated. For example, a 34 ng DNA/μL can require 2.94 μL for 100 ng DNA. In an aspect, LB broth (Miller's modification) (e.g., 25 g) can be added to dH₂O (e.g., 1 L), which can be sterilized by autoclave. It can be cooled to room temperature. 50 mg/mL ampicillin stock (e.g., about 2 mL) can be added for a final concentration of about 100 μg/mL ampicillin. It can be stored at 4° C. in the cold room. In an aspect, LB+100 μg/mL ampicillin (e.g., 6 mL) can be added to tubes (e.g., 15 mL Falcon tubes, for example, n=10). In an aspect, frozen SOC medium can be thawed by hand and/or can be heated to 37° C. in a water bath. In an aspect, a 1.5 mL Eppendorf tube and a 0.2 cm electroporation cuvette can be placed on ice for ≥10 minutes prior to the electroporation. In an aspect, a disclosed One Shot TOP10 electrocompetent E. coli tube can be removed from the −80° C. freezer and can be thawed on ice ≥5 minutes.

In an aspect, the following can be done on ice. 100 ng of DNA (obtained as set forth above) can be transferred into a chilled Eppendorf tube. Thawed electrocompetent bacteria (e.g., 25 μL) can be added. Everything can be mixed by gently flicking the tube and then the tube can be placed on ice. In an aspect, the mix is not pipetted up and down. Using a long, gel loading pipette tip, the DNA and electrocompetent bacteria solution can be transferred to a pre-chilled 0.2 cm cuvette. In an aspect, a disclosed solution can fill the entire bottom of the cuvette, can cover the whole lengths of the electrodes, and can ensure there are no bubbles. In an aspect, a cuvette can be removed from ice and the sides can be wiped (e.g., with a Kimwipe) to remove moisture. Immediate electroporation can be performed with an exponential pulse of 1.6 kV, 200Ω, and 25 μF. In an aspect, if popping is heard and a low time constant is received, then arcing occurred. Arcing can be due to moisture on the sides of the cuvette, air bubbles, or solution not covering electrodes. If arcing occurred, then the electroporation can be repeated. Then, warmed SOC (e.g., 250 μL) can be immediately added to the cuvette. The SOC can be mixed by pipetting up and down gently. The next steps can be done as quickly as possible to avoid bacterial death and lack of growth. Bacteria solution (e.g., 27 μL) can be added to one tube (e.g., Falcon tube) and incubated for 1 hour at 37° C. with shaking at ≥225 rpm. Bacteria solution (e.g., 27 μL) can be added to each of the tubes (e.g., Falcon tubes, n=10) with LB+100 μg/mL (e.g., 6 mL). Falcon tubes can be incubated overnight at 37° C. with shaking at ≥225 rpm.

In an aspect, a disclosed method of directing evolution of one or more biomolecules can comprise screening bacteria and purifying circular plasmid DNA. In an aspect of a disclosed method of directing evolution of one or more biomolecules, bacteria are screened and circular plasmid DNA is purified.

In an aspect, screening bacteria and purifying circular plasmid DNA can comprise one or more of the following components/ingredients/instruments: a UVP ChemStudio 615 Imaging System (Analytik Jena US) or equivalent to image fluorescence of liquid cultures (e.g., a plate reader that measures the fluorescence of liquid cultures), a QIAprep Spin Miniprep Kit (Qiagen, 27104), Nuclease-free H₂O, and NanoDrop One^{C} UV-Vis Spectrophotometer (ThermoFisher, ND-ONEC-W). In an aspect, a disclosed ingredient/component/instrument can be substituted for another industry accepted equivalent for that ingredient/component/instrument.

In an aspect, screening bacteria and purifying circular plasmid DNA can comprise removing from the shaker and capturing fluorescent images of the liquid cultures (e.g., using a UVP Imager or similar instrument with excitation and emission filters). In an aspect, a disclosed set of filters can be changed to accommodate different fluorescent proteins and/or fluorophores. In an aspect, exposure time, gain, and light intensity can be adjusted for maximum fluorescent intensity. Care should be taken to ensure that the fluorescence signal can be separated. In an aspect, a disclosed excitation and a disclosed emission filters can be 644-657 and 660-720 nm, respectively, for a disclosed smURFP. In an aspect, a disclosed circular plasmid DNA can be purified from E. coli using, for example, a QIAprep Spin Miniprep Kit. The overnight bacteria culture (e.g., 6 mL) can be pelleted and can be centrifuged at 16,000 RCF for 1 min. In an aspect, the liquid can be discarded. Buffer P1 (e.g., 250 μL) can be added and can be vortexed to resuspend the bacteria cells. In an aspect, care should be taken to ensure that all bacteria are suspended, and that no bacterial remain on the bottom of the tube or in large aggregates that are visible by the eye. For example, if a disclosed plasmid expresses a fluorescent protein, then the pellet can be colored. For example, for smURFP, this can be blue. For NIR-FPs, this can be dark green.

In an aspect, bacteria can be suspended in Buffer P1 and can be transferred to an Eppendorf tube (e.g., a 2 mL tube). In an aspect, Buffer P2 (e.g., 250 μL) can be added and the tube can be inverted (e.g., about 5 times). Buffer N3 (e.g., 350 μL) can be quickly added and the tube can be inverted (e.g., 3-6 times) to neutralize the pH. In an aspect, this step can be done as soon as possible. In an aspect, a tube should not be incubated for minutes as 5 minutes can be maximum time allowed. Care should be taken to complete this step as quickly as possible.

In an aspect, a tube can be centrifuged (e.g., at 16,000 RCF for about 10 min at room temperature). Supernatant (e.g., 800 μL) can be removed and can be added it to a spin column (e.g., QIAprep 2.0). Care should be taken not to dislodge the pellet and to ensure that the supernatant is clear. It can be centrifuged (e.g., 16,000 RCF for 1 min) and flow-through can be discarded. The column can be washed with Buffer PE (e.g., 750 μL) and can be centrifuged (e.g., 16,000 RCF for 1 min) and the flow-through can then be discarded. Centrifuging (e.g., 1 min at 16,000 RCF) can be done to remove the excess buffer. In an aspect, a spin column can be transferred to a clean Eppendorf tube (e.g., 1.5 mL) and H₂O (e.g., 50 μL of 72° C.) can be applied directly to the column matrix. Again, centrifuging (e.g., 16,000 RCF for 1 min) can be done to elute DNA. Pure water (H₂O) can be used to avoid salts and other additives that can inhibit subsequent PCR and sequencing.

In an aspect, the concentration of a disclosed circular plasmid DNA can be measured using the NanoDrop One^{C} UV-Vis Spectrophotometer (or a similar instrument).

In an aspect, for example, equal parts of all samples (e.g., 10 samples) can be mixed for a total of 10 μg DNA. To do this, the volume of each sample needed for 1.0 μg of DNA can be calculated. After combining the circular plasmid DNA samples, the samples can be mixed thoroughly by vortexing and can be quantified using NanoDrop One^{C} UV-Vis Spectrophotometer (or a similar instrument).

In an aspect, a disclosed DNA mixture (e.g., 10 μg) can be used as the circular plasmid DNA template for the second round of error prone PCR. In an aspect, a disclosed circular plasmid DNA template obtained after a second round of error prone PCR can be the template for a third round of error prone PCR. In an aspect, a disclosed method of directing evolution of one or more biomolecules can comprise repeating one or more times a disclosed error prone PCR mutagenesis to prepare a DNA library step, repeating a disclosed electroporation of linear plasmid DNA to circularize plasmid DNA step, and repeating a disclosed screening and circular plasmid DNA purification step. In an aspect, repeating one or more times a disclosed error prone PCR mutagenesis to prepare a DNA library step, a disclosed electroporation of linear plasmid DNA to circularize plasmid DNA step, and repeating a disclosed screening and circular plasmid DNA purification step can increase the number of mutations in the polynucleotide encoding the one or more biomolecules of interest. For example, in an aspect, circular plasmid DNA obtained after a first round of error prone PCR can be used as a template for a second round of error prone PCR, which can then serve as a template for a third round of error prone PCR. In an aspect, each round of error prone PCR can increase the number of mutations in the polynucleotide encoding the one or more biomolecules of interest. In an aspect, after 3 rounds of error prone PCR, the circular plasmid DNA can be used to electroporate cells such as, for example, HEK cells (as described below).

In an aspect, when a biomolecule of interests comprises a fluorescent protein, later rounds of directed evolution can comprise a single error prone PCR because multiple mutations are not necessary to significantly enhance the fluorescence.

In an aspect, a disclosed method of directing evolution of one or more biomolecules can comprise growing mammalian cells. In an aspect of a disclosed method of directing evolution of one or more biomolecules, mammalian cells are grown. In an aspect, a disclosed method of directing evolution of one or more biomolecules can comprise growing cells of interest. In an aspect of a disclosed method of directing evolution of one or more biomolecules, cells of interest are grown.

In an aspect, growing mammalian cells or cells of interest can comprise one or more of the following components/ingredients/instruments: 10 cm Tissue Culture-treated dishes (Eppendorf, 0030702115), Low Glucose (1 g/L) Dulbecco's Modified Eagle's Medium (DMEM) (Millipore Sigma, D6046-1 L), Fetal bovine serum (FBS) (Gibco, 10437-036), 100× Penicillin-Streptomycin (Pen-Strep) (Gibco, 15140-122), 100× GlutaMAX (Gibco, 35050-061), TrypLE Express Enzyme (1×), no phenol red (trypsin) (Gibco, 12604-013), and Dulbecco's Phosphate-Buffered Saline (DPBS) without Ca²⁺ and Mg²⁺ (Gibco, 14190250) with 20 mM HEPES, pH 7.4+1 mM EDTA. In an aspect, a disclosed ingredient/component/instrument can be substituted for another industry accepted equivalent for that ingredient/component/instrument.

In an aspect, disclosed growth media can be made by adding 10% FBS (e.g., 100 mL), 1× Pen-Strep (e.g., 10 mL), and 1× GlutaMAX (e.g., 10 mL) directly to low glucose (1 g/L) DMEM media (e.g., 1 L) bottle to make DMEM 1 g/L glucose+10% FBS+1× Pen/Strep+1× GlutaMax. Care should be taken to ensure the sterility of all reagents by, for example, only opening in a biosafety cabinet (BSC). For example, a disclosed buffer made in the lab or glassware used in a disclosed experiment can be sterilized by filtration or autoclaved before use in a disclosed cell culture. Low glucose DMEM can be used to grow cells slower for less splitting. In an aspect, high glucose (4.5 g/L) DMEM media can be used for faster growth and can be used after electroporation.

In an aspect, HEK293A cells can be stored frozen in cryovials under liquid nitrogen (N₂) in a cell bank. To start growing HEK293A cells, a cryovial can be removed out of the liquid N₂ and can be quickly placed in a 37° C. water bath to thaw as fast as possible. Shaking by hand in the water bath can help to thaw faster. Once thawed, cells (e.g., 1 mL) can be transferred to a Falcon tube (e.g., 15 mL) containing warm growth media (e.g., 10 mL). The cells can be pelleted by centrifugation (e.g., 90 RCF for 10 minutes) and the media can be aspirated. This step can be necessary to remove the DMSO in the freezing media, small, lysed dead cells, and molecules.

Fresh, warm growth media (e.g., 10 mL) can be added to the Falcon tube and the cell pellet can be resuspended by gently pipetting up and down with a serological pipette. The entire volume can be transferred to a 10 cm TC-treated dish and incubated (e.g., at 37° C. with 5% CO₂ and 95% H₂O). CO₂ can be used to maintain the pH at 7.4. Humidity can be used to stop media evaporation. In an aspect, if the phenol red is orange, then the media is acidic. If the phenol red is dark purple, then the media is basic. Cells can be split at 90-100% confluency. Freshly thawed cells can, for example, grow slower. The cells can be checked daily to track the rate of growth and confluency. When the cells are ready to split, DPBS, growth media, trypsin, and a new 10 cm cell dish can be placed in the BSC.

The growth media can be aspirated from the dish. Aspirating can take place from the side of the dish to avoid removing adherent cells. DPBS without Ca²⁺ and Mg²⁺ with 20 mM HEPES pH 7.4+1 mM EDTA (e.g., 10 mL) can be immediately to the dish. In an aspect, DPBS (e.g., 10 mL) can be added slowly to the side to avoid removing adherent cells. This solution can be used to remove divalent cations with EDTA that can inhibit cleavage by trypsin.

The solution can be gently mixed in the dish (e.g., typically three times each: clockwise, counter-clockwise, front-to-back, and side-to-side). DPBS can be removed by aspiration. In an aspect, TrypLE Express Enzyme (e.g., 300 μL) or similar enzyme can be quickly added and can be mixed by tilting the dish to cover the surface of the dish. The dish can be placed into the incubator for ˜5 min. During incubation, fresh growth media (e.g., 9 mL) can be placed in a new, sterile 10 cm TC-treated dish.

In an aspect, cells can be removed from the incubator and fresh growth media (e.g., 10 mL) can be added. The growth media can add divalent cations to inhibit trypsin. Usually, the same volume of growth media can be added to calculate the dilution. The cells and growth media can be pipetted up and down, known as trituration, until there are no more visible clumps of cells or adherent cells. In an aspect, this step can be done for obtaining single cells to allow room for cell growth.

The dissociated cell solution (e.g., 1 mL) can be added to growth media (e.g., 9 mL) in a new dish. The solution was gently mixed in the dish, typically three times each (e.g., clockwise, counter-clockwise, front-to-back, and side-to-side). The cells can be placed in the incubator (e.g., 37° C. with 5% CO₂ and 95% H₂O). The remaining cell solution can be disposed by aspiration.

At 100% confluency, the 10 cm dishes can contain about 8.0×10⁶ cells to about 9.0×10⁶ cells. In an aspect, the 10 cm dishes can contain ˜8.8×10⁶ cells. In an aspect, HEK293A cells can be electroporated as described below and can use ˜3.0×10⁶ cells per electroporation sample and ˜6.0×10¹ cells for wild-type HEK293A cells to use as a background sample. If the experiments electroporate two DNA samples, then ≥6.6×10⁶ cells can be used, for example.

In an aspect, extra cells can be preferred in the event that a dish is contaminated or grows slowly. For example, two dishes at 100% confluency can ensure enough cells on the day of the electroporation. To do this, 2 dishes can be used during incubation described above. In an aspect, one or more dishes can be grown.

In an aspect, a disclosed method of directing evolution of one or more biomolecules can comprise electroporating mammalian cells with circular plasmid DNA. In an aspect, a disclosed method of directing evolution of one or more biomolecules can comprise electroporating cells of interest with circular plasmid DNA. In an aspect of a disclosed method of directing evolution of one or more biomolecules, circular plasmid DNA can be lipofected into mammalian cells. In an aspect of a disclosed method of directing evolution of one or more biomolecules, circular plasmid DNA can be lipofected into cells of interest.

In an aspect, electroporating cells or cells of interest with circular plasmid DNA can comprise one or more of the following components/ingredients/instruments: the circular plasmid DNA from the third error prone PCR described above, High Glucose (4.5 g/L) DMEM (Gibco, 11965092), 35 mm Tissue Culture-treated Dishes (Eppendorf, 0030700112) or a 6 well plate (Eppendorf, 0030720113), Opti-MEM Reduced Serum Media (Gibco, 31985-070), Countess 3 FL Automated Cell Counter (ThermoFisher, A50299), Countess Cell Counting Chamber Slides (ThermoFisher, C10228), 0.4% Trypan Blue Solution (Gibco, 15250-061), 10× Hanks' Balanced Salt Solution (HBSS) (Gibco, 14065-056), Gene Pulser Xcell Total Electroporation system (BioRad, 1652660), and 0.2 cm electroporation cuvettes (BioRad, 1652086). In an aspect, a disclosed ingredient/component/instrument can be substituted for another industry accepted equivalent for that ingredient/component/instrument.

In an aspect, the third error prone PCR circular plasmid DNA (described supra) can be electroporated into HEK293A cells (or other cells of interest) for protein expression. In an aspect, error prone PCR can be random, and each reaction can generate a unique DNA library that encodes billions of unique biomolecules. In an aspect, no two DNA libraries can be the same. The goal of mixing ten error prone PCR samples can be to cover as much protein sequence space as possible. In an aspect, all mutations cannot be assessed at one time, and multiple amino acids mutations can be required. For example, in an aspect, for the small Ultra-Red Fluorescent Protein, 20 amino acid mutations were necessary to create a bright, far-red fluorescent protein over 12 selection rounds (see, e.g., U.S. Pat. No. 8,932,859 and Rodriguez E A, et al. (2016) Nat. Methods. 13:763-769). This protocol can use electroporation to efficiently introduce DNA into millions of cells, and selection can be performed on transiently expressed proteins. In an aspect, the transient expression for 2 days can be done to ensure the circular plasmid DNA remains for circular plasmid DNA purification.

In an aspect, the procedure can be done by creating viruses using viral plasmids and transducing the mammalian cells with viruses. However, viruses increase the selection time and elevate the biosafety level of the procedure. If using a FACS after transduction with a virus, care should be taken to ensure to use enhanced safety precautions and notify the technician. Many flow core facilities require that the virus be completely gone from samples, which can take 1-2 weeks with additional testing. Recovery of the mutated biomolecule would require mRNA recovery, reverse transcription to ssDNA, PCR amplification to dsDNA, and molecular biology to subclone into a plasmid. Some viruses incorporate viral RNA into the genome as dsDNA, which would require PCR amplification or other means to recover the evolved protein DNA sequence. In an aspect, a biosafety cabinet (BSC) can be used to perform all cell culture work.

After the third error prone PCR (described supra), the ten circular plasmid DNA libraries from the final PCR can be mixed in equal parts. The mixture can be 10 μg total and can use 1.0 μg of each circular plasmid DNA library with ten libraries. The concentration of the mixed circular plasmid DNA library can be measured using the NanoDrop One^{C} UV-Vis Spectrophotometer (or other instrument), and the volume needed for 2 μg of circular plasmid DNA can be calculated.

High glucose (4.5 g/L) DMEM+10% FBS+1× Pen/Strep+1× GlutaMax (e.g., 2 mL) can be added to six 35 mm cell culture dishes or a 6 well plate and can be incubated (e.g., 37° C. with 5% CO₂ and 95% H₂O). In an aspect, the circular plasmid DNA can be electroporated into HEK293A cells (or other cells of interest) can be divided into five equal 0.4 mL volumes. In an aspect, one dish can be used as a control with HEK293A cells without DNA (or other cells without DNA) to look at the background fluorescence. Arcing can occur during electroporation and can result in a loss of the DNA. In an aspect, splitting the HEK293A cells into 5 equal volumes can avoid the risk of losing all the DNA in a single electroporation.

In an aspect, when the 10 cm TC dishes disclosed supra are 95-100% confluent, the growth media can be aspirated from the dish. Aspirating can take place from the side of the dish to avoid removing adherent HEK293A cells (or other cells). DPBS without Ca²⁺ and Mg²⁺ with 20 mM HEPES pH 7.4+1 mM EDTA (e.g., 10 mL) can be immediately to the dish. In an aspect, DPBS (e.g., 10 mL) can be added slowly to the side to avoid removing adherent HEK293A cells (or other cells). This solution can be used to remove divalent cations with EDTA that can inhibit cleavage by trypsin. The solution can be gently mixed in the dish (e.g., typically three times each: clockwise, counter-clockwise, front-to-back, and side-to-side). DPBS can be removed by aspiration. In an aspect, TrypLE Express Enzyme (e.g., 300 μL) or similar enzyme can be quickly added and can be mixed by tilting the dish to cover the surface of the dish. The dish can be placed into the incubator for ˜5 min. Removing the adherent HEK293A cells (or other mammalian cells) can be repeated.

In an aspect, cells can be removed from the incubator and fresh growth media (e.g., 10 mL) can be added. The growth media adds divalent cations to inhibit trypsin. Usually, the same volume of growth media can be added to calculate the dilution. The cells and growth media can be pipetted up and down until there are no more visible clumps of cells or adherent cells. In an aspect, this step can be done for obtaining single cells to allow room for cell growth.

The non-adherent HEK293A cells can be transferred to a 15 mL Falcon tube and the cells can be pelleted by centrifugation (e.g., 90 RCF for 5 min). This step can change the media. In an aspect, when there are ≥2 dishes of cells, the cells can be combined into a 50 mL Falcon tube. Care should be taken to no use a higher RCF because greater RCF can pellet dead cells and can also kill the living cells. In an aspect, DMEM+1 g/L glucose+10% FBS+1× Pen/Strep+1× GlutaMax can be aspirated completely and X mL of Opti-MEM (as described above) can be gently added. Single cell suspensions can be created by pipetting up and down with a 5 mL or 10 mL serological pipette or large opening P1000 tip.

In an aspect, a serological pipette or a P1000 tip can be used because smaller tips can lyse the cells. In an aspect, the volume of Opti-MEM can depend on the confluency of the cells and the desired number of dishes. In an aspect, the concentration can be determined by counting the cells using a cell counter (Countess II FL Automated Cell Counter, ThermoFisher). The cell solution can be kept ≥6.0×10⁶ cells/mL, which can be the desired final concentration of cells (described infra). In a 0.5 mL Eppendorf tube, HEK293A cells (e.g., 5.0 μL) can be mixed in Opti-MEM with 0.4% Trypan Blue Solution (e.g., 5.0 μL). In an aspect, cells with trypan blue (e.g., 10 μL) can be added into the cell counter or hemocytometer slide. In an aspect, the concentration of cells/mL can be determined. The desired concentration can be ≥6.0×10⁶ cells/mL. In an aspect, the volume of Opti-MEM required to dilute the cell solution can be calculated to be to 6.0×10⁶ cells/mL. In an aspect, the Opti-MEM can be added to the cells and can be mixed by pipetting. In an aspect, 600,000 cells can be required for each 35 mm dish. If the concentration is 6.0×10⁶ cells/mL, then 100 L can be used for each dish. In an aspect, concentrations slightly greater or less can be used, but changing volume greatly can change the efficiency of circular plasmid DNA delivery to the HEK293A cells (or other cells).

In an aspect, in a 1.5 mL Eppendorf tube, circular plasmid DNA (e.g., 2 μg) obtained as described above can be mixed with 6.0×10⁶ cells/mL (e.g., 100 μL) obtained above. Mixing can occur via gently flicking or using a P1000 pipette. In an aspect, the entire DNA and cell solution can be carefully pipetted using a gel loading tip into a 0.2 cm electroporation cuvette stored at room temperature. The solution can be filled the entire bottom of the cuvette, the whole lengths of the electrodes can be covered, and there can be no bubbles. The 0.2 cm electroporation cuvette package and lid should not be opened outside of a BSC. Care should be taken to keep the cuvette sterile to avoid contamination. When removed from the BSC, care should be taken to cap the cuvette. In an aspect, the cap can be placed on the electroporation cuvette and can be removed from the BSC for electroporation. The sides of the cuvette can be wiped (e.g., with a KimWipe) and the DNA can be electroporated into the HEK293A cells (or other cells) using the Gene Pulser Xcell Total Electroporation system parameters: Gap size=0.2 cm, pulse type is a square wave, pulse amplitude=110 V, and pulse width=25 ms.

In an aspect, a square wave pulse can be used for mammalian cells. Voltage and pulse width can be optimized for efficient DNA delivery. In an aspect, Bio-Rad Manual and website can be consulted when using other cell types. Electroporation can be used to achieve high efficiency transfection. In an aspect, lipofectamine or other polyamine lipid transfections can be used, which can be optimized. Viruses can enhance transduction but can require the production of viruses that increase evolution time and can require more stringent safety measures. A loud pop and low time constant are indicative of arcing. Arcing is usually caused by high salt, air bubbles, not covering the entire length of the electrodes, or a defective cuvette (rare). Here, arcing electricity went through the air rather than the solution, and the sample should be thrown out. The solutions can be divided into 5 samples to avoid losing all the DNA. If two consecutive samples arc, care should be taken to ensure the parameters and gap size are correct. The electroporation cuvette can be immediately placed on ice to enhance the efficiency of DNA electroporation. Care should be taken to not let the cuvette chill for >1 min.

In an aspect, the cuvette can be wiped with 70% ethanol before placing it into the BSC. In an aspect, a 35 mm cell culture dish or 6 well plate with pre-warmed high glucose (4.5 g/L) DMEM+10% FBS+1× Pen/Strep+1× GlutaMax (described above) can be removed from the incubator and can be placed in the BSC. Using a gel loading tip, the entire electroporated circular plasmid DNA and cell solution can be removed and can be added to the 35 mm cell culture dish. The solution can be gently mixed in the dish three times each (e.g., clockwise, counter-clockwise, front-to-back, and side-to-side). The cells can be incubated (e.g., 37° C. with 5% CO₂ and 95% H₂O). HEK293A cells can initially non-adherent and can require time to adhere to the plastic.

The cells can be incubated for 2 days to allow biomolecule production for screening. The incubation time can be changed, but this can evolve proteins that require that incubation time for efficient production. In an aspect, two days is typically the time needed to achieve proper fluorescent protein expression and chromophore attachment to see on any fluorescent microscope. Longer incubations require testing to ensure the circular plasmid DNA is still present and purified from the cells.

In an aspect, dead cells typically are non-adherent or floating and can be removed by aspiration. Dividing HEK293A cells or other cells can become non-adherent during cell division. If all cells are dead or floating, care should be taken to use high glucose DMEM with GlutaMAX and FBS, have the correct cell number, and cells are added to prewarmed high glucose DMEM. In an aspect, floating cells can be screened by FACS using side and forward scatter to select properly sized HEK293A cells.

In an aspect, a disclosed method of directing evolution of one or more biomolecules can comprise subjecting the mammalian cells to a sorting process. In an aspect of a disclosed method of directing evolution of one or more biomolecules, the mammalian cells are subjected to a sorting process. In an aspect, a disclosed method of directing evolution of one or more biomolecules can comprise subjecting HEK293A cells to a sorting process. In an aspect of a disclosed method of directing evolution of one or more biomolecules, the HEK293A cells are subjected to a sorting process.

In an aspect, subjecting mammalian cells (e.g., HEK293A cells) to cell sorting can comprise one or more of the following components/ingredients/instruments: EVOS FL Auto II Cell Imaging Epifluorescence Microscope (ThermoFisher, AMAFD2000) or comparable Inverted Fluorescence Microscope with long working distance objectives for imaging through the plastic, Cy5 EVOS LED Light Cube (ThermoFisher, AMEP4956) or equivalent excitation light and filters. For evolving smURFP or Cy5-like fluorescent proteins, Cy5.5 EVOS LED Light Cube (ThermoFisher, AMEP4973) or equivalent excitation light and filters for evolving IFP or Cy5.5-like fluorescent proteins, TrypLE Express Enzyme (1×), no phenol red (Trypsin) (Gibco, 12604-013), Low Glucose (1 g/L) Dulbecco's Modified Eagle's Medium (DMEM) (Millipore Sigma, D6046-1 L), PBS, pH 7.4 (Gibco, 10010-023), 0.5 M EDTA, 1 M HEPES (Gibco, 15630106), FBS (Gibco, 10437036), FACS Transportation Media [(1×PBS, pH 7.4+1 mM EDTA+25 mM HEPES+1% FBS (500 mL 1×PBS+1 mL 0.5 M EDTA+12.5 mL 1 M HEPES+5 mL FBS)], and FACS Recovery Media: [1×HBSS (Gibco, 14065-056)+2 g/L D-(+)-glucose+20 mM HEPES, pH 7.4 (Imaging Media)+10% FBS+1× Pen-Strep (500 mL 1×HBSS+2 g/L glucose+20 mM HEPES, pH 7.4)+50 mL FBS+5 mL 100× Pen-Strep)]. In an aspect, a disclosed ingredient/component/instrument can be substituted for another industry accepted equivalent for that ingredient/component/instrument.

In an aspect, the initial screening protocol can be for far-red and near-infrared fluorescent proteins to confirm cell expression. In an aspect, the initial screening protocol can screen protein expression by gel electrophoresis, Western blot, or immunofluorescence. Nucleic acid expression can be verifiable by Northern/Southern blot, fluorescence in situ hybridization (FISH), or reverse transcription and PCR amplification with Sanger sequencing or Next Generation Sequencing (NGS). Cell activity or inhibition can be monitored using stable cell lines that express fluorescent sensors for calcium (GCaMP) or other ions. Enzyme sensors, such as kinase and GPCR, can be used to assay the inhibition or activation of the enzymes or receptors. The evolution of biomolecules that reduce gas, such as CO₂, can be performed with CO₂ sensors in a closed tissue culture dish. In an aspect, the directed evolution can be applied to fluorescent proteins and disclosed screening protocol can include fluorescence. In an aspect, the directed evolution can be directed to any biomolecule and the disclosed screening protocol can include a non-fluorescence characteristic and/or property.

After 2 days, fluorescent protein expression can be verified by fluorescence imaging on an inverted microscope with long working distance or low numerical aperture (NA) objectives for imaging through plastic (EVOS FL Auto 2 with Cy5 and Cy5.5 LED light cubes). Images can confirm fluorescent protein expression and identified spectral properties of fluorescent proteins.

The same exposure, gain, and parameters can be used when taking images with the Cy5 and Cy5.5 LED light cubes. A brightfield or white light image can be taken to show the total number of cells in the image to calculate the approximate transfection efficiency.

Images can be taken using the 10×, 20×, and 40× objectives. The 10× and 20× objectives can estimate the transfection efficiency for the entire dish. The 40× objective can be useful for publication quality images and identification of the cellular location of fluorescent proteins. The dish lid can avoid removal so as to keep the cells sterile. Care should be taken not to spill media when placing the dish on the microscope.

If long working distance objectives are not available, then fluorescence can be screened by adding cells to glass bottom dishes (described above). In an aspect, #1.5 glass can allow for imaging on fluorescence microscopes with high numerical apertures and water/oil immersion objectives. Poly-D-Lysine or collagen coating can be recommended for adherent cells. Here, MatTek 35 mm glass bottom dishes (P35GC-1.5-14-C) can be used. In the BSC, the media from the 5 dishes can be combined in a 15 mL Falcon tube. The step can be done to keep all the non-adherent cells that are floating. The floating cells can be a mixture of dividing, dead, and non-adherent cells for FACS sorting. Dead cells can be fluorescent and contain circular plasmid DNA. In early selection rounds, more cells can be better to increase the amount of circular plasmid DNA purified. The FACS side and front scatter gating can remove small cells and large cell clumps. In later rounds, if most of the cells are non-adherent, or the selection requires the cells to be adherent, the floating cells can be discarded to screen adherent cells.

In an aspect, trypsin (e.g., 100 μL) can be added to each dish, the dish can be tilted to coat the entire surface evenly, and can be incubated (e.g., 37° C. with 5% CO₂ and 95% H₂O for ˜5 min). In the BSC, low glucose (1 g/L) DMEM+10% FBS+1× Pen/Strep+1× GlutaMax (e.g., 1 mL) made as described above can be added to stop the Trypsin by the addition of divalent cations. The solution can be triturated by pipetting up and down to remove the adherent cells from the dish and can break up cell clusters. The cell solution can be added to the same 15 mL Falcon tube obtained above. In an aspect, a serological pipette or a P1000 tip can be used to avoid cell lysis. The cells can be pelleted by centrifuge (e.g., 90 RCF for 5 min). Media can be aspirated carefully so as not to dislodge the pellet. Centrifugation can be at 90 RCF avoided pelleting small, partial cells and to prevent killing the cells. In an aspect, the cell pellet can be resuspended in 1 mL of prewarmed, 37° C. FACS Transportation Media.

The FACS Transportation Media can be useful when traveling or keeping the cells outside of an incubator for long periods of time. PBS and HEPES can maintain the pH at 7.4 without CO₂. EDTA can be added to chelate divalent cations to avoid cell clumping. 1% FBS can be added to promote cell health. While HEK293A cells can survive at room temperature, other, more delicate cells can be transferred in a heated cooler. In an aspect, the cells can be placed in a Styrofoam secondary container to reduce temperature changes. Gloves, extra FACS Transportation Media to dilute cells, 1.5 mL Eppendorf Tubes with 50 μL of FACS Recovery Media for each selection, P1000 tips, micropipette, and spare 1.5 mL Eppendorf tubes with 50 μL of FACS Recovery Media can be gathered. The FACS Recovery Media contains divalent cations, FBS, Glucose, and HEPES to keep the cells alive and avoid DNA degradation after FACS. Penicillin and streptomycin can be added to prevent bacterial growth after FACS, which can cause DNA degradation. Most FACS instruments may be contaminated and are not in a BSC. Technicians often recommend the addition of Pen/Strep to avoid microbial growth.

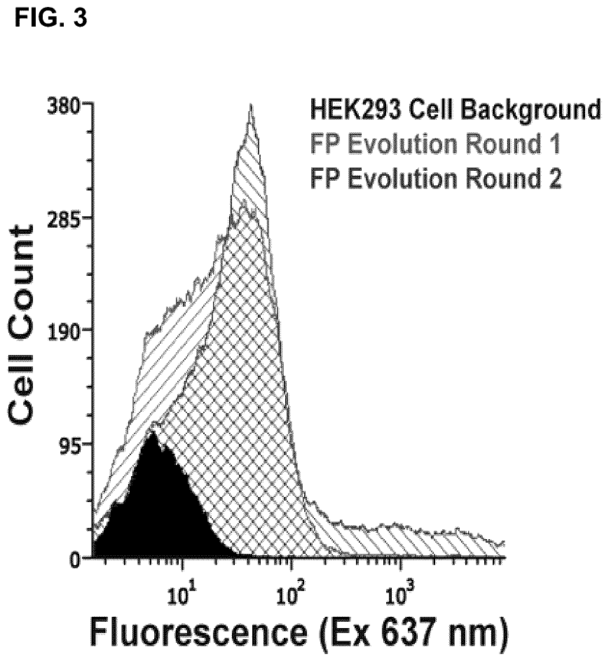

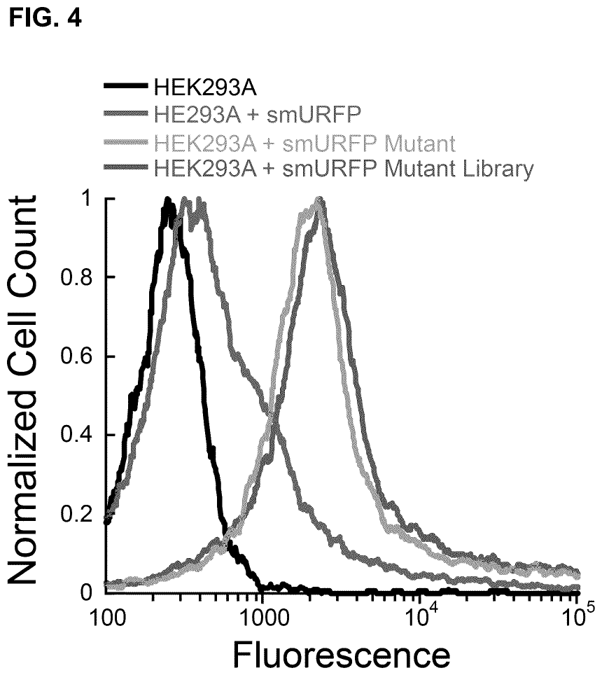

In an aspect, before fluorescence-activated cell sorting (FACS), the cells can be mixed by pipetting up and down many times to remove clumps that may have formed during travel. Before FACS, the cells can be filtered through a 70 μm nylon mesh (Falcon, 2350) to remove large cell clumps to avoid clogging the FACS tubing and nozzle. Fluorescent proteins can be selected using a 637 nm laser with APC or Cy5 filters. Redshifted fluorescent proteins can be selected with a 637 nm laser with APC-Cy5.5 or APC-Cy7 filters. In an aspect, 200,000 cells can be run in flow cytometry mode to choose the gate (FIG. 3 and FIG. 4). Typically, from 1-10% of the highest fluorescent cells depending on the total cell number can be gated. Ideally, ≥20,000 cells from a selection of 2,000,000 cells are preferred for circular plasmid DNA purification. Collected cells can be recorded for each run. In an aspect, ˜200,000 wild-type HEK293A cells can be run to visualize the autofluorescence with identical travel and parameters.

In an aspect, a disclosed method of directing evolution of one or more biomolecules can comprise lysing the mammalian cells and recovering the circular plasmid DNA. In an aspect of a disclosed method of directing evolution of one or more biomolecules, the mammalian cells can be lysed, and the circular plasmid DNA can be recovered. In an aspect, a disclosed method of directing evolution of one or more biomolecules can comprise lysing the cells of interests and recovering the circular plasmid DNA. In an aspect of a disclosed method of directing evolution of one or more biomolecules, the cells of interest can be lysed, and the circular plasmid DNA can be recovered.

In an aspect, lysing cells (e.g., mammalian or otherwise) and recovering circular plasmid DNA can comprise one or more of the following components/ingredients/instruments: a QIAprep Spin Miniprep Kit (Qiagen, 27106), a Zymo DNA Clean & Concentrator-5 Kit (Zymo Research, D4013), Nuclease-free H₂O, and a NanoDrop One^{C} UV-Vis Spectrophotometer (ThermoFisher, ND-ONEC-W). In an aspect, a disclosed ingredient/component/instrument can be substituted for another industry accepted equivalent for that ingredient/component/instrument. In an aspect, the cells can be pelleted by centrifugation (e.g., at 500 RCF for 5 min). The FACS Recovery Media supernatant can be aspirated off by using a pipette with a P200 tip, for example, to ensure total removal. Care should be taken to avoid dislodging the pellet. In an aspect, a small number of cells can be in the pellet, and the RCF can be increased slightly to obtain a larger pellet. Care should be taken to avoid high-speed centrifugation that can lyse the cells and can lose the circular plasmid DNA in the supernatant.

In an aspect, the circular plasmid DNA can be purified using, for example, a QIAprep Spin Miniprep Kit. HEK293A cell pellet (or other mammalian cell pellet) can be resuspended in Buffer P1 (e.g., 250 μL) and vortexed. Care should be taken to ensure that the pellet is dislodged, and the solution can be homogenous. Buffer P2 (e.g., 250 μL) can be added and can be mixed by inverting several times (e.g., about 5 times). In an aspect, Buffer N3 (e.g., 350 μL) can be quickly added and inverted several times (e.g., about 5 times) to neutralize the pH. In an aspect, this step can be done as soon as possible. Care should be taken not to incubate for 5 minutes. In an aspect, 5 minutes is the maximum time allowed and instead, this step should be done as fast as possible.

In an aspect, the solution can then be centrifuged (e.g., 16,000 RCF for 10 min at room temperature). Supernatant (e.g., about 800 μL) can be removed and added to a spin column (e.g., a Zymoclean Spin column). Care should be taken not to dislodge the pellet, and to keep the supernatant clear. Centrifuging (e.g., 16,000 RCF for 1 min) can be done and flow-through can be then discarded. Care should be taken to use a Zymoclean Spin column and not, for example, a QIAprep Spin column. All buffers used in the purification process can be from a Zymo DNA Clean & Concentrator-5 Kit. The column can be twice washed (e.g., 200 μL wash buffer each time for 400 μL wash buffer total), and can then be centrifuged (e.g., 1 min at 16,000 RCF) after each wash. The flow-through can be discarded. Again, centrifuging (e.g., 1 min at 16,000 RCF) can be done to remove the excess buffer. In an aspect, the spin column can be transferred to a clean Eppendorf tube (e.g., 1.5 mL) and dH₂O (e.g., 10 μL of 72° C.) can applied directly to the column matrix. Centrifuging (e.g., 16,000 RCF for 1 min) can be used to elute DNA. Pure, nuclease-free water (H₂O) can be used to avoid salts and other additives that can inhibit subsequent PCR and sequencing.

In an aspect, the purified circular plasmid DNA can be quantified using, for example, the NanoDrop One^{C} UV-Vis Spectrophotometer (or a similar instrument). In an aspect, the total amount of DNA recovered can be ˜30 ng to ˜50 ng. In an aspect, the volume of solution needed for 10 ng of circular plasmid DNA purified from HEK293A cells (or other cells) can be calculated.

In an aspect, a disclosed method of directing evolution of one or more biomolecules can comprise electroporating bacteria and plating electroporated bacteria. In an aspect of a disclosed method of directing evolution of one or more biomolecules, wherein the bacteria can be electroporated and the electroporated bacteria can be plated.

In an aspect, electroporating bacteria and plating electroporated bacteria can comprise one or more of the following components/ingredients/instruments: Ampicillin (Millipore Sigma, A1593), Luria broth (LB) agar (Millipore Sigma, L2897) 10 cm plates Ampicillin, Super Optimal broth (e.g., 1,500 μg/mL) with Catabolite repression (SOC) media is Hanahan's Broth or SOB medium (Millipore Sigma, H8032) with 20 mM D-(+)-Glucose (Millipore Sigma, G8270), One Shot TOP10 electrocompetent E. coli (ThermoFisher, C66455), Gene Pulser Xcell Total Electroporation system (BioRad, 1652660), and 0.2 cm electroporation cuvettes (BioRad, 1652086). The high ampicillin concentration can help avoid satellite colonies. In an aspect, a disclosed ingredient/component/instrument can be substituted for another industry accepted equivalent for that ingredient/component/instrument.

Gene expression of β-lactamase can appear upregulated after purification of circular plasmid DNA from HEK293A cells. A single colony can express enough β-lactamase to remove ampicillin for surrounding, untransformed bacteria to survive. The high concentration can be characterized as not necessary for liquid culture because of shaking. Liquid media with 100 μg/mL ampicillin can be used. In an aspect, before starting, the LB/agar plates with ampicillin can be prepared at least one day prior. Extra plates for plating can be poured and can be stored. Extra plates can be stored for >6 months at 4° C. in the dark.

In an aspect, each mutagenic DNA library can use ˜20 LB/agar plates. The number of plates can change depending on the number of bacterial colonies. In an aspect, preparing the plates can comprise removing the pre-autoclaved, 500 mL LB/agar jar from the refrigerator. In an aspect, the top can be unscrewed and can be placed on top to avoid pressure buildup and LB/agar boiling into the microwave. Microwaving can occur, in an aspect, for 30 seconds and the LB/agar mixture can be swirled until fully dissolved. In an aspect, the dissolved LB/agar jar can be put into a 55° C. water bath for ˜45 min. This step can be performed to cool the mixture to 55° C. for ampicillin addition and can avoid agar solidification. In an aspect, the LB/agar jar can be left longer than 45 min in the water bath. In an aspect, the temperature can be checked at 55° C. with an infrared thermometer. Ampicillin (e.g., 100 μg/mL) from 50 mg/ml ampicillin stocks can be added. For a 500 mL LB/agar jar, in an aspect, the 50 mg/mL ampicillin stock solution (e.g., 1 mL) can be added. The solution can be mixed thoroughly by swirling. Care should be taken not to shake vigorously to avoid air bubbles. In an aspect, the LB/agar jar can be stored at 55° C. to avoid agar solidification. In an aspect, a 500 mL LB/agar jar can provide enough material for about or at least a sleeve of 20 plastic bacteria plates.

In an aspect, the LB/agar mixture can be carefully poured into the plates to avoid creating bubbles. This step can be done near an open flame to avoid contaminants in the air. In an aspect, a poured plate can be stored at room temperature and covered to avoid ampicillin degradation by light. In an aspect, the frozen SOC can be warmed by hand until completely thawed. In an aspect, the SOC can be heated in a 42° C. dry bath block and can remain in the dry bath block until needed.

In an aspect, a 1.5 mL Eppendorf and a 0.2 cm electroporation cuvette can be placed on ice for ≥10 minutes prior to electroporation. In an aspect, a single tube of One Shot TOP10 electrocompetent E. coli from the −80° C. freezer can be placed on ice ≥5 minutes before electroporation. In an aspect, the electrocompetent E. coli can be fully thawed before use. Care should be taken not to thaw by pipetting up and down. In an aspect, the tube can be quickly flicked to gently mix but can avoid a temperature change.

In an aspect, the following steps can be performed on ice. The circular plasmid DNA (e.g., 10 ng) can be purified from HEK293A cells grown as described above into the chilled Eppendorf tube and freshly thawed electrocompetent bacteria (e.g., 15 μL) can be added. Care should be taken not to pipette up and down. In an aspect, the solution in the tube can be gently flicked and can be placed on ice.

In an aspect, a gel loading pipette tip can be used to transfer the circular plasmid DNA and bacteria solution into the pre-chilled 0.2 cm cuvette. Care should be taken to ensure the solution fills the bottom of the cuvette and the whole length of the electrodes. Care should be taken that there are no bubbles. In an aspect, both sides of the cuvette can be wiped (e.g., with a KimWipe, for example) to remove moisture. If the cuvette is wet, then arcing can occur during electroporation. The cuvette can be placed into the electroporation system and can be electroporated with an exponential pulse with voltage=1.6 kV, resistance=200Ω, and capacitance=25 uF. In an aspect, an exponential pulse can be used to electroporate the disclosed circular plasmid DNA into bacteria. A loud pop and low time constant can indicate arcing, and the electricity did not flow through the sample. If arcing occurs, then the tube can be discarded and the electroporation can be repeated.

In an aspect, a prewarmed SOC (e.g., 250 μL) can be immediately added to the cuvette and can be mixed by pipetting up and down slowly. In an aspect, the disclosed bacteria in SOC can be added to one tube (e.g., Falcon tube) and incubated for 1 hr at 37° C. with shaking at ≥225 rpm. In an aspect, the disclosed bacteria in SOC can be spread on the LB/Agar plates with 100 μg/mL ampicillin using sterile, autoclaved glass beads in the following amounts: 2 plates with 40 μL, 5 plates with 20 μL, 8 plates with 10 μL, and 5 plates with 1 μL, generating a total of 20 plates. In an aspect, care should be taken to spread all the bacteria and SOC solution. In an aspect, different volumes can be used to ensure that the plates are not overgrown with a lawn or tiny bacteria colonies. In an aspect, care should be taken to ensure that there is enough space to select a single colony to avoid mixing the circular plasmid DNA from two or more bacterial colonies. In an aspect, purified circular plasmid DNA from each evolution round can have different transformation efficiencies, and the dilutions can ensure that plates contain individual colonies. In an aspect, the plates can be incubated overnight at 37° C. in a dry incubator with the LB/Agar side facing up.

In an aspect, a disclosed method of directing evolution of one or more biomolecules can comprise lipofecting circular plasmid DNA into mammalian cells and screening lipofected mammalian cells. In an aspect, a disclosed method of directing evolution of one or more biomolecules can comprise lipofecting circular plasmid DNA into HEK293A cells and screening lipofected HEK293A cells. In an aspect of a disclosed method of directing evolution of one or more biomolecules, wherein the circular plasmid DNA can be lipofected into mammalian cells and the lipofected mammalian cells can be screened. In an aspect of a disclosed method of directing evolution of one or more biomolecules, wherein the circular plasmid DNA can be lipofected into HEK293A cells and the lipofected HEK293A cells can be screened.

In an aspect, lipofecting circular plasmid DNA into mammalian cells and screening lipofected mammalian cells can comprise one or more of the following components/ingredients/instruments: UVP ChemStudio 615 Imaging System (Analytik Jena US) or equivalent to image fluorescence of liquid cultures. A plate reader may be used to measure the fluorescence of liquid cultures, LB liquid media with 100 μg/mL ampicillin, QIAprep Spin Miniprep Kit (Qiagen, 27104), and Nuclease-free H₂O, NanoDrop One^{C} UV-Vis Spectrophotometer (ThermoFisher, ND-ONEC-W). In an aspect, a disclosed ingredient/component/instrument can be substituted for another industry accepted equivalent for that ingredient/component/instrument.

In an aspect, images of the bacterial colonies can be captured using a UVP Imager or similar instrument with excitation and emission filters of 644-657 and 660-720 nm, respectively. The exposure time, gain, and light intensity can be adjusted for maximum fluorescent intensity and to ensure that the fluorescence signal was not saturated. For example, in an aspect, these filters can be for smURFP and can be changeable for other fluorescent proteins. The images can be opened in NIH ImageJ (Schindelin J, et al. (2012) Nat Methods. 9:676-682; Schneider C A, et al. (2012) Nat. Methods. 9:671-675) to select the brightest fluorescent colonies. The “Brightness/Contrast” can be used to adjust the image histogram. In an aspect, the “Find Maxima” function can be used and the threshold value can be adjusted until 10-15 colonies are marked as maxima to locate the brightest fluorescent colonies. In an aspect, the selection can be in mammalian cells, and the brightest fluorescent proteins in mammalian cells can be dimmer in bacterial cells. In an aspect, an equal amount of bright and less fluorescent single colonies can be picked to avoid evolving fluorescent proteins that fluorescence well in bacteria.

In an aspect, selected colonies can be picked using a pipette tip or autoclaved toothpick. A single colony can be carefully touched and the tip can be placed into a 15 mL Falcon tube. In an aspect, a separate tube can be used for each colony. In an aspect, LB with 100 μg/mL ampicillin (e.g., 6 mL) can be added to each 15 mL Falcon tube and incubated (e.g., 37° C. with shaking at ≥225 rpm overnight).

In an aspect, after overnight incubation, the bacterial screening step and the circular plasmid DNA purification step can be repeated to fluorescently image the liquid cultures and the circular plasmid DNA can be purified. In an aspect, the concentration of circular plasmid DNA can be quantified using the NanoDrop One^{C} UV-Vis Spectrophotometer (or a similar instrument).

In an aspect, a disclosed method of directing evolution of one or more biomolecules can comprise lipofecting circular DNA into mammalian cells and screening lipofected mammalian cells. In an aspect of a disclosed method of directing evolution of one or more biomolecules, wherein the circular DNA can be lipofected into mammalian cells and the lipofected mammalian cells can be screened. In an aspect, a disclosed method of directing evolution of one or more biomolecules can comprise lipofecting circular DNA into HEK293A cells and screening lipofected mammalian cells. In an aspect of the disclosed method of the directed evolution of one or more biomolecules, the circular DNA can be lipofected into HEK293A cells and the lipofected mammalian cells can be screened.

In an aspect, lipofecting circular DNA into mammalian cells (e.g., HEK293A cells) and screening lipofected mammalian cells can comprise one or more of the following components/ingredients/instruments: Lipofectamine 3000 (ThermoFisher, L3000-015), 96-Well Black/Clear Bottom Plate, TC Surface (Thermo Fisher Scientific, 165305), HEK293A Cells (ThermoFisher, R70507), Opti-MEM (Gibco, 31985-070), Biliverdin dimethyl ester (BVMe₂) (Frontier Scientific, B610-9), Imaging Media: 1×HBSS (Gibco, 14065-056)+2 g/L D-(+)-glucose+20 mM HEPES, pH 7.4, and Varioskan LUX Fluorescence Plate Reader (ThermoFisher). In an aspect, a disclosed ingredient/component/instrument can be substituted for another industry accepted equivalent for that ingredient/component/instrument.

In an aspect, HEK293A cells can be grown to 95-100% confluency as described above. In an aspect, HEK293A cell solution (e.g., 200 μL) can be added to a well in a 96-well plate. This process can be continued to match twice the number of purified circular plasmid DNA samples from previous steps. In an aspect, 9-12 extra wells can be added (i) in the event that a well did not grow, (ii) to have control wells without DNA, and (iii) to have wells with starting fluorescent protein DNA (discussed below) for background autofluorescence. The cells can be allowed to adhere overnight with incubation (e.g., 37° C. with 5% CO₂ and 95% H₂O).

In an aspect, 2 wells can be used for each purified DNA described above. In an aspect, if there are 15 DNA samples, then 30 wells can be used. In an aspect, an additional 9-12 wells of control cells can be employed—including 3 wells transfected with the original circular plasmid DNA (e.g., smURFP) without the addition of BVMe₂, 3 wells transfected with the original circular plasmid DNA (e.g., smURFP) with the addition of 1 μM BVMe₂, and 3 wells with mammalian cells (e.g., HEK293A cells) without DNA transfection. In an aspect, the minimum number of wells can be 9, and the extra 3 can substitute if another well does not adhere. In an aspect, for this experiment, 39 wells can be used with 3 additional wells.

In an aspect, after overnight incubation, the adherent mammalian cells (e.g., HEK293A cells) can be transfected with DNA using lipofection following the Lipofectamine 3000 protocol. All steps can be performed in the BSC to keep solutions and cells sterile. In an aspect, for each well, two (2) 1.5 mL Eppendorf tubes can be used. In an aspect, circular plasmid DNA (e.g., 100 obtained above) can be mixed with Opti-MEM (e.g., 5 μL) and P3000 (e.g., 0.2 μL) in a 1.5 mL tube. In the second 1.5 mL Eppendorf tube, Lipofectamine 3000 (e.g., 0.15 μL) can be mixed with Opti-MEM (e.g., 5 μL). The solutions can be vortexed separately and the two tubes can be combined to form DNA complexes with polyamine lipids. The mixture can be incubated for 15 minutes in the BSC without UV light. Care should be taken to carefully add to each well by using the side of the well. Avoid dislodging the adherent cells from the bottom of the well. Incubation can be overnight (e.g., 37° C. with 5% CO₂ and 95% H₂O). In an aspect, 2 replicates for each circular plasmid DNA sample can be used, and the volumes can be doubled to circular plasmid DNA (e.g., 200 ng)+Opti-MEM (e.g., 10 μL)+P3000 (e.g., 0.4 μL). In an aspect, a stock solution of Opti-MEM and Lipofectamine 3000 can be made for all wells in a single tube. For 39 wells, Lipofectamine 3000 (e.g., 5.85 μL) can be added to Opti-MEM. Remember (e.g., 195 μL) to mix components well.

Care should be taken not close the BSC glass shield. Closing the shield can activate the UV light, which can destroy the DNA. In an aspect, when the technician leaves the hood during the incubation, place a “No UV sign” can be placed on the glass shield. After 24 hr, for the mammalian cells (e.g., HEK293A cells) expressing the evolved protein (e.g., smURFP), the media can be aspirated and can be replaced with 1 μM BVMe₂ (e.g., 0.2 mL) in growth media (2 mL DMEM+10% FBS+1× Pen/Strep+1× GlutaMax+0.2 μL of 5 mM BVMe₂ in DMSO). Incubation can be done overnight (e.g., at 37° C. with 5% CO₂ and 95% H₂O). The DMSO can be dense and can sink to the bottom of the tube. The solution can be mixed well.

After 24 hr, the media can be aspirated from the wells and can be replaced imaging media (1×HBSS (Gibco, 14065-056)+2 g/L D-(+)-glucose+20 mM HEPES, pH 7.4) (e.g., 200 μL) of Imaging media can be added to 3 unused wells to measure autofluorescence of the growth media. Care should be taken not to aspirate the cells off the bottom of the wells. The HBSS can be gently added to the side of the well. Adding to the side can avoid dislodging adherent cells.

Imaging media can be used to maintain pH=7.4 without CO₂. Glucose can be added to ensure healthy cells. This media can be suitable for use for 3 hr-5 hr. Longer incubations can be toxic to cells, and other buffers can be used for long-term imaging. In an aspect, fluorescently imaging the cells can be performed as described supra. In an aspect, Varioskan LUX Fluorescence Plate Reader (ThermoFisher) (or a similar instrument) can be used to automatically screen the fluorescence of the transiently expressed fluorescent proteins in mammalian cells (e.g., HEK293A cells). The average fluorescence and absorbance of adherent mammalian cells (e.g., HEK293A cells) can be measured using the following parameters.

Fluorescence 1: Excitation 642 nm & Emission 672 nm (far-red fluorescence), Fluorescence 2: Excitation 685 nm & Emission 715 nm (near-infrared fluorescence), Excitation bandwidth: 12 nm for both, Optics: Read from the bottom (Record from adherent cells), Dynamic range: Low, Measurement time: 1000 ms, Measurement points: All points on well, Absorbance 1: 280 nm, Absorbance 2: 380 nm, Absorbance 3: 642 nm, Use transmittance: No, Pathlength correction: Yes, Measurement time: 1000 ms. In an aspect, background subtraction can be selected for every measurement taken. In an aspect, all the wells can be labeled properly to perform background subtraction automatically. New circular plasmid DNA samples can be labeled “Unknown”, wells with untransfected mammalian cells (e.g., HEK293A cells), wells with mammalian cells (e.g., HEK293A cells) expressing smURFP with 1 μM BVMe₂, mammalian cells (e.g., HEK293A cells) expressing smURFP with 1 BVMe₂ can be “Controls”, and the wells with imaging media alone can be “Background”.

In an aspect, after the plate reader finished the measurements, the 96 well black plate can be placed in a BSC. The media can be aspirated from the wells and the 96 well black plate can be sprayed with 70% ethanol to sterilize the wells. Care should be taken to avoid spraying ethanol in unused wells, which can be used in future experiments. The average fluorescence measurements can be compared at both wavelengths to the HEK293A cells expressing smURFP (controls) to select the circular plasmid DNA that produced fluorescence brighter than smURFP without BVMe₂. The epifluorescence images can be compared to each other and against the control images. DNA expressing fluorescent proteins with the highest average fluorescence can be selected for Sanger sequencing. The plate reader can be used to verify the FACS selection. A single cell can show highly variable fluorescence, and this screen can measure the fluorescence from many cells to get an average fluorescence that can be more accurate.

Purified circular plasmid DNA can be selected and sent for Sanger sequencing to GENEWIZ (or a similar service provider). In an aspect, circular plasmid DNA can be diluted to a concentration of 80 ng/μL DNA in nuclease-free H₂O (e.g., 10 μL). GENEWIZ (or similar) primers can be used to sequence from the CMV promoter (pDuEx2 and pCDNA3) or another other promoter employed in the plasmid. Sequencing can be usually 800-1,000 bp. Custom primers can be used with 20-30 bases before the desired sequencing region because the start of the fluorescent chromatograph can generally be saturated with fluorescence from the initial PCR terminations.

In an aspect, a disclosed method of directing evolution of one or more biomolecules can comprise identifying amino acid mutations by sequencing and analyzing the DNA. In an aspect of a disclosed method of directing evolution of one or more biomolecules, wherein the DNA can be sequenced and can be analyzed to identify amino acid mutations. In an aspect of a disclosed method of directing evolution of one or more biomolecules, wherein the amino acid mutations can be identified when the DNA is sequenced and analyzed.

In an aspect, this protocol can analyze Sanger sequencing to determine amino acid changes in the one or more newly produced biomolecules of interest. In an aspect, a disclosed DNA nucleotide mutation or mutations can be translated into a protein and can be compared to the parent protein to identify amino acids changes that alter the property of interest (such as, for example, fluorescence). In an aspect, the selected circular plasmid DNA can be analyzed and those proteins that have unique and multiple amino acid changes can be selected for subsequent directed evolution.

In an aspect, care should be taken not to select DNA with mutations in non-coding regions or silent mutations that do not change the amino acid. For example, if there are no mutations, then select non-coding and silent mutations over the initial DNA. In an aspect, these mutations can enhance transcription or translation and can increase the probability of amino acid change in subsequent rounds. This protocol describes the method for selecting a desired protein (e.g., fluorescent protein) to be used as the template in the error prone PCR for the next round of directed evolution.

In an aspect, identifying amino acid mutations by sequencing and analyzing the DNA can comprise one or more of the following components/ingredients/instruments: a Plasmid Editor (APE) made by M. Wayne Davis (freely available at https://jorgensen.biology.utah.edu/wayned/ape/) or use a similar DNA analysis software to align the new DNA and protein sequences. In an aspect, mutated nucleotides in the new proteins (e.g., fluorescent proteins) can be located using the starting protein and PCR template alignment. Highlighting or case changing can be used to locate the mutated nucleotide. In an aspect, all genes can be translated to obtain the amino acid sequences. In an aspect, mutated nucleotides in the new protein amino acid sequence can be identified.

In an aspect, whether the nucleotide mutations change the amino acid or are “silent mutations” due to the redundancy of the genetic code can be determined. In an aspect, silent mutations are undesired because the amino acid sequence remains the same, but the nucleotide change can enhance protein expression. In an aspect, desirable nucleotide mutations can cause an amino acid change. In an aspect, error prone PCR can be biased towards specific nucleotides, so if there are no amino acid changes, multiple rounds of error prone PCR can be performed to enhance the mutation frequency. In an aspect, adding high concentrations of dNTPs with high concentrations of Mg²⁺ and Mn²⁺ can also increase the frequency of mutations. In an aspect, increasing the concentration of these components can decrease the overall yield of the PCR.

In an aspect, a new protein with unique and multiple amino acid changes can be used as the template for the next round of directed evolution. In an aspect, multiple protein sequences can be mixed, and when used in equal proportions, can avoid bias in the library. In an aspect, if there are no amino acid changes, error prone PCR can be repeated with multiple PCR reactions to enhance the mutation frequency. If the fluorescent protein sequences have silent mutations, use these as templates for the error prone PCR because these can increase the probability of changing the amino acid in subsequent rounds.

### C. Miscellaneous

Disclosed herein is a system for directed evolution of at least one biomolecule of interest, the system comprising a DNA polymerase fused to a DNA binding protein, wherein the DNA polymerase does not comprise any editing activity; and a plasmid comprising at least one promoter for transcription and translation, and a polynucleotide sequence encoding the at least one biomolecule of interest to be evolved, wherein the system evolves the at least one biomolecule of interest by introducing one or more mutations into the polynucleotide sequence encoding the at least one biomolecule of interest.

In an aspect, a disclosed entire plasmid can be amplified by the DNA polymerase fused to a DNA binding protein. In an aspect, a disclosed polymerase fused to a DNA binding protein can amplify at least about 0.1 kilobases of DNA. In an aspect, a disclosed DNA polymerase fused to a DNA binding protein can amplify about 0.1 kilobases of DNA to about 15 kilobases of DNA. In an aspect, a disclosed DNA polymerase fused to a DNA binding protein can amplify more than about 15 kilobases of DNA. In an aspect, a disclosed DNA polymerase fused to a DNA binding protein can amplify more than about 15 kilobases of DNA after optimization of at least one aspect of the system wherein the at least one aspect of the system comprises reaction temperature, extension time, number of cycles, amount of DNA primers, or any combination thereof. In an aspect, a disclosed plasmid can comprise at least two biomolecules of interest to be evolved. In an aspect, a disclosed plasmid can comprise at least two or more biomolecules of interest to be evolved. In an aspect, disclosed biomolecules of interest can comprise nucleic acids, oligonucleotides, oligonucleosides, proteins, peptides, enzymes, amino acids, lipids, organic molecules, small molecules, or any combination thereof. In an aspect, disclosed proteins can comprise proteins endogenous to a host cell, proteins exogenous to a host cell, or any combination thereof. In an aspect, disclosed proteins endogenous to a host cell, proteins exogenous to a host cell, or any combination thereof increase the number of small molecules including inorganic salts in a cell, excreted by a cell, outside of a cell, or any combination thereof. In an aspect, disclosed small molecules can comprise one or more inorganic salts. In an aspect, disclosed organic molecules can comprise organic molecules native to a host cell, organic molecules not native to a host cell, or any combination thereof. In an aspect, disclosed organic molecules can comprise intracellular organic molecules, extracellular organic molecules, or any combination thereof. In an aspect, disclosed organic molecules can comprise amino acids. In an aspect, disclosed biomolecule of interest can comprise antisense oligonucleotides (ASOs), small interfering RNAs (siRNAs), silencing RNAs, DNA aptamers, RNA aptamers, transfer RNAs (tRNAs), ribosomal RNAs (rRNAs), long non-coding RNAs (lncRNAs), microRNAs (miRNAs), small nucleolar RNAs (snoRNAs), small nuclear RNAs (snRNAs), piwi-interacting RNAs (piRNAs), mRNAs, gene-editing guide RNAs (gRNAs), or any combination thereof. In an aspect, disclosed biomolecule of interest can comprise full-length antibodies, antigen binding fragments of full-length antibodies, Fab fragments, single chain antibodies (scFv), diabodies, triabodies, minibodies, nanobodies, single-domain antibodies, camelids, or any combination thereof. In an aspect, disclosed biomolecule of interest can comprise one or more enzymes. In an aspect, disclosed enzymes can perform novel protein, RNA, and/or DNA modifications; elevate native small molecules; create novel small molecules not present inside cells; degrade plastic or waste products to form useful molecules; use carbon dioxide or other gases to create synthetically useful molecules and reduce greenhouse gases; or any combination thereof. In an aspect, a disclosed the system can be used in metabolomic engineering, creating one or more synthetic organisms for production of unnatural amino acids for creation of one or more proteins with new functions, producing one or more biological circuits for molecule sensing and memory storage, genetic reprogramming, or any combination thereof. In an aspect, a disclosed plasmid can comprise bacterial expression plasmids, mammalian expression plasmids, insect expression plasmids, viral expression plasmids, species-specific host organism expression plasmids, or any combination thereof. In an aspect, a disclosed system can comprise comprises a mutation efficiency of at least about 1-in-200 base pairs. In an aspect, a disclosed system can comprise a mutation efficiency of about 1-in-200 base pairs. In an aspect, disclosed method can further comprise a host cell comprising the plasmid comprising at least one promoter for transcription and translation. In an aspect, a disclosed host cell can comprise bacteria, yeast, insect cells, mammalian cells, species-specific host cells, or any combination thereof. In an aspect, a disclosed host cell can comprise a human cell. In an aspect, a disclosed system can evolve the at least one biomolecule of interest by introducing one or more mutations into the polynucleotide sequence encoding the at least one biomolecule of interest, the linear plasmid DNA is circularized into a circular plasmid DNA in bacteria. In an aspect, a disclosed circular plasmid DNA can comprise a bacterial library.

Disclosed herein is a method of directed evolution of at least one biomolecule of interest to be evolved, the method comprising preparing a circular plasmid DNA comprising at least one promoter for transcription and translation, and a polynucleotide sequence encoding the at least one biomolecule of interest to be evolved; amplifying the entire circular plasmid DNA into an entire linear plasmid DNA by performing a single error prone PCR in the presence of a DNA polymerase fused to a DNA binding protein, wherein the DNA polymerase does not comprise any editing activity; evolving the at least one biomolecule of interest by introducing mutations into the polynucleotide sequence encoding the at least one biomolecule of interest during the single error prone PCR by addition of increased concentrations of magnesium, manganese, and deoxynucleotide triphosphates during the PCR amplification of the entire circular plasmid DNA into linear plasmid DNA containing mutations throughout the DNA sequence; circularizing the linear plasmid DNA into a circular plasmid DNA in bacteria, incorporating the circular plasmid DNA into a host cell, assessing at least one evolved biomolecule for one or more resultant activity(ies) of interest in the host cell. In an aspect, a disclosed method can further comprise sequencing of the at least one evolved biomolecule to identify one or more mutations. In an aspect, a disclosed method can further comprise subjecting the at least one evolved biomolecule having at least one or more resultant activity(ies) of interest attributed to the at least one or more mutations identified by sequencing, to a disclosed method of directed evolution.

In an aspect, a disclosed evolved biomolecule can be subjected to a disclosed method of directed evolution about two or more times. In an aspect, amplifying the entire circular plasmid DNA into an entire linear plasmid DNA by performing a single error prone PCR in the presence of the DNA polymerase fused to a DNA binding protein can generate a DNA library encoding at least about 100 evolved biomolecules. In an aspect, amplifying the entire circular plasmid DNA into an entire linear plasmid DNA by performing a single error prone PCR in the presence of the DNA polymerase fused to a DNA binding protein can generate a DNA library encoding about one million to about one trillion evolved biomolecules. In an aspect, a disclosed plasmid can comprise at least two or more biomolecules of interest to be evolved. In an aspect, a disclosed host cell can comprise bacteria, yeast, insect cells, mammalian cells, species-specific host cells, or any combination thereof. In an aspect, a disclosed host cell can comprise a human cell.

In an aspect, disclosed biomolecules of interest can comprise nucleic acids, oligonucleotides, oligonucleosides, proteins, peptides, enzymes, amino acids, lipids, organic molecules, small molecules, or any combination thereof. In an aspect, disclosed proteins can comprise proteins endogenous to a host cell, proteins exogenous to a host cell, or any combination thereof. In an aspect, disclosed proteins endogenous to a host cell, proteins exogenous to a host cell, or any combination thereof can increase the number of small molecules including inorganic salts in a cell, excreted by a cell, outside of a cell, or any combination thereof. In an aspect, disclosed small molecules can comprise one or more inorganic salts. In an aspect, disclosed organic molecules can comprise organic molecules native to a host cell, organic molecules not native to a host cell, or any combination thereof. In an aspect, disclosed organic molecules can comprise intracellular organic molecules, extracellular organic molecules, or any combination thereof. In an aspect, disclosed organic molecules can comprise amino acids. In an aspect, disclosed biomolecule of interest can comprise antisense oligonucleotides (ASOs), small interfering RNAs (siRNAs), silencing RNAs, DNA aptamers, RNA aptamers, transfer RNAs (tRNAs), ribosomal RNAs (rRNAs), long non-coding RNAs (lncRNAs), microRNAs (miRNAs), small nucleolar RNAs (snoRNAs), small nuclear RNAs (snRNAs), piwi-interacting RNAs (piRNAs), mRNAs, gene-editing guide RNAs (gRNAs), or any combination thereof. In an aspect, disclosed biomolecule of interest can comprise full-length antibodies, antigen binding fragments of full-length antibodies, Fab fragments, single chain antibodies (scFv), diabodies, triabodies, minibodies, nanobodies, single-domain antibodies, camelids, or any combination thereof.

In an aspect, disclosed biomolecule of interest can comprise one or more enzymes. In an aspect, disclosed enzymes can perform novel protein, RNA, and/or DNA modifications; elevate native small molecules; create novel small molecules not present inside cells; degrade plastic or waste products to form useful molecules; use carbon dioxide or other gases to create synthetically useful molecules and reduce greenhouse gases; or any combination thereof.

In an aspect, a disclosed method can be used in metabolomic engineering, creating one or more synthetic organisms for production of unnatural amino acids for creation of one or more proteins with new functions, producing one or more biological circuits for molecule sensing and memory storage, genetic reprogramming, or any combination thereof. In an aspect, translating the plasmid into linear plasmid DNA by performing PCR in the presence of the DNA polymerase fused to a DNA binding protein can generate a biomolecule DNA library. In an aspect, translating the plasmid into linear plasmid DNA by performing PCR in the presence of the DNA polymerase fused to a DNA binding protein can generate an antibody DNA library. In an aspect, translating the plasmid into linear plasmid DNA by performing PCR in the presence of the DNA polymerase fused to a DNA binding protein can generate a gRNA DNA library, a CRISPR DNA library, a Cas DNA library, a Zinc Finger DNA library, a TALEN DNA library, a restriction enzyme DNA, or any combination thereof. In an aspect, a disclosed gRNA DNA library, CRISPR DNA library, Cas DNA library, Zinc Finger DNA library, TALEN DNA library, restriction enzyme DNA, or any combination thereof can be transcribed in vitro or inside the host cell. In an aspect, a disclosed gRNA DNA library can be used in combination with CRISPR/Cas proteins to site-specifically cut genomic DNA to knock out, to change nucleotides, to change amino acids upon mRNA translation, or to knock in added DNA to screen for cellular phenotypes. In an aspect, a disclosed CRISPR DNA library, Cas DNA library, Zinc Finger DNA library, TALEN DNA library, restriction enzyme DNA, or any combination thereof can be transcribed and translated into at least one protein.

In an aspect, a disclosed CRISPR DNA library, the Cas DNA library, the Zinc Finger DNA library, the TALEN DNA library, the restriction enzyme DNA, or any combination thereof can be translated into at least one protein that is evolved to reduce off-target cutting and enhance specificity, to create new PAM sequence recognition, to produce improved dsDNA and/or nickase breakage with enhanced kinetics to speed the reaction, to create new variants for producing knock out, to create nucleotide changes, to knock in with added DNA, to introduce site-specific modification of genomic or recombinant DNA, or any combination thereof. In an aspect, a disclosed in vitro or cell lysate can be used to produce the biomolecules.

In an aspect, a disclosed method of assessing the at least one evolved biomolecule for one or more resultant activity(ies) of interest in the host cell can comprise FACS, gel electrophoresis, ELISA, high-throughput plate reader fluorescence or absorbance assays, high-content imaging of single cells, mass spectrometry proteomics, metabolomics, kinemics, omics analysis, Western blot, Northern blot, Southern blot, fluorescence in situ hybridization (FISH), spatial transcriptomics, fluorescence microscopy, fixed expansion microscopy, electron microscopy, reverse transcription and PCR amplification, Sanger sequencing, Next Generation Sequencing (NGS), or any combination thereof.

In an aspect, a disclosed plasmid comprising at least one promoter for transcription and translation and the polynucleotide sequence encoding the at least one biomolecule of interest to be evolved, further can comprise a polynucleotide sequence encoding for at least one sensor, wherein the at least one sensor comprises a fluorescent protein, a small molecule binding protein sensor, a GPCR-based sensor, an enzyme activity sensor, a protein modification sensor, an RNA modification sensor, a DNA modification sensor, a biomolecule modification sensor, a protein-protein interaction (PPi) sensor, a fluorescent ubiquitination-based cell cycle indicator (FUCCI), a cell membrane voltage sensor, or any combination thereof.

In an aspect, a disclosed small molecule binding protein sensor can detect calcium, magnesium, zinc, a small inorganic molecule, glucose, lactose, cyclic adenosine monophosphate, dopamine, acetylcholine, a small molecule sugar, a neurotransmitter, a molecule used inside cells, or any combination thereof.

In an aspect, a disclosed GPCR-based sensor can detect calcium, magnesium, zinc, a small inorganic molecule, glucose, lactose, cyclic adenosine monophosphate, dopamine, acetylcholine, a small molecule sugar, a neurotransmitter, a molecule used inside cells, or any combination thereof.

In an aspect, a disclosed plasmid comprising at least one promoter for transcription and translation, and the polynucleotide sequence encoding the at least one biomolecule of interest to be evolved, can further comprise a polynucleotide sequence encoding at least one fluorescent protein of interest, wherein the at least one fluorescent protein of interest is evolved to improve brightness, enhance biophysical properties, bind new chromophore molecules, create reversible photo-activatable fluorescent proteins, create irreversible photo-activatable fluorescent proteins, or any combination thereof.

In an aspect, a disclosed plasmid comprising at least one promoter for transcription and translation, and the polynucleotide sequence encoding the at least one biomolecule of interest to be evolved further comprises a polynucleotide sequence encoding an optogenetic element, the optogenetic element can comprising a channel rhodopsin, a halorhodopsin, a light-oxygen voltage (LOV) domain, a phytochrome light-activated enzyme, a biomolecule activated by light, a biomolecule activated or deactivated by light, or any combination thereof, wherein the at least one biomolecule of interest can be evolved to alter kinetics, light modulation and/or enhance performance.

In an aspect, a disclosed biomolecule of interest can be evolved for use as one or more research tools. In an aspect, a disclosed research tools can comprise research tools to modulate protein expression within a cell or a model organism, to perform gene editing within a cell or a model organism, to modulate small molecule concentrations within a cell or a model organism, to modulate enzyme activity within a cell or a model organism, to bind small molecules to visualize location, to identify a protein by specific epitope binding, or any combination thereof. In an aspect, a disclosed model organism can comprise a vertebrate, an invertebrate, or any combination thereof. In an aspect, a disclosed model organism can comprise bacteria, yeast, fish, worms, insects, rodents, or any combination thereof. In an aspect, a disclosed biomolecule of interest can be evolved for use as one or more treatments or prophylactics of a disease or a disorder.

In an aspect, disclosed treatments or prophylactics of a disease or a disorder can comprise modulating protein number, creating chaperone proteins for elevated proteins at proper cellular location, creating tRNAs that suppress nonsense and frameshift mutations to produce full-length genes, producing non-coding RNAs to alter and engineer cellular functions, identification of unknown functions of RNA, creating immunotherapies, creating small molecules, creating therapeutic proteins, or any combination thereof.

In an aspect, a disclosed disease or the disorder can comprise cancer. In an aspect, a disclosed disease or the disorder can comprise a vision disorder, a sleep disorder, a circadian disorder, or any combination thereof. In an aspect, a disclosed biomolecule of interest can be evolved for use as one or more treatments or prophylactics administered to a subject having or suspected of having a disease or a disorder. In an aspect, a disclosed subject having or suspected of having a disease or a disorder can be a human patient. In an aspect, a disclosed human patient can be diagnosed as having or is suspected of having cancer. In an aspect, a disclosed human patient can be diagnosed as having or is suspected of having a vision disorder, a sleep disorder, a circadian disorder, or any combination thereof.

Disclosed herein is a kit comprising a DNA polymerase fused to a DNA binding protein, wherein the DNA polymerase does not comprise any editing activity; a host-specific plasmid comprising at least one promoter for transcription and translation; and at least one reaction solution. In an aspect, a disclosed reaction solution can comprise PCR buffers, increased concentrations of magnesium, manganese, and deoxynucleotide triphosphates, nuclease-free water, or any combination thereof. In an aspect, a disclosed host-specific plasmid can comprise a bacterial expression plasmid, a mammalian expression plasmid, an insect expression plasmid, a viral expression plasmid, or another host organism expression plasmid. In an aspect, a disclosed kit can comprise chemically or electro-competent bacterial cells for the circularization of linear plasmid DNA libraries.

### VII. EXAMPLES

The novel and inventive compositions and methods described herein remove the molecular biology steps and expresses biomolecules in mammalian cells. Mammalian cells increase the number of biomolecules in a larger cell volume for more efficient fluorescent-activated cell sorting. The procedure evolves biomolecules in mammalian cells to purify large quantities of biomolecules without bacterial endotoxins and other antigens that could elicit an immune response. This novel and inventive method removes the need to use restriction enzymes to cut the PCR gene product and vector, dephosphorylate the vector, and a ligase to subclone the cut PCR product into the dephosphorylated vector. Removing these molecular biology steps reduces the time required to prepare biomolecule libraries for directed evolution and screening. The circular plasmid DNA is directly purified from mammalian cells to avoid reverse-transcription of mRNA, PCR amplification of the ssDNA, and subcloning with multiple steps into a new plasmid DNA. The Examples that follow are illustrative of specific aspects of the invention, and various uses thereof. They set forth for explanatory purposes only and are not to be taken as limiting the invention.

### Example 1

### Error Prone PCR Mutagenesis for DNA Library Preparation

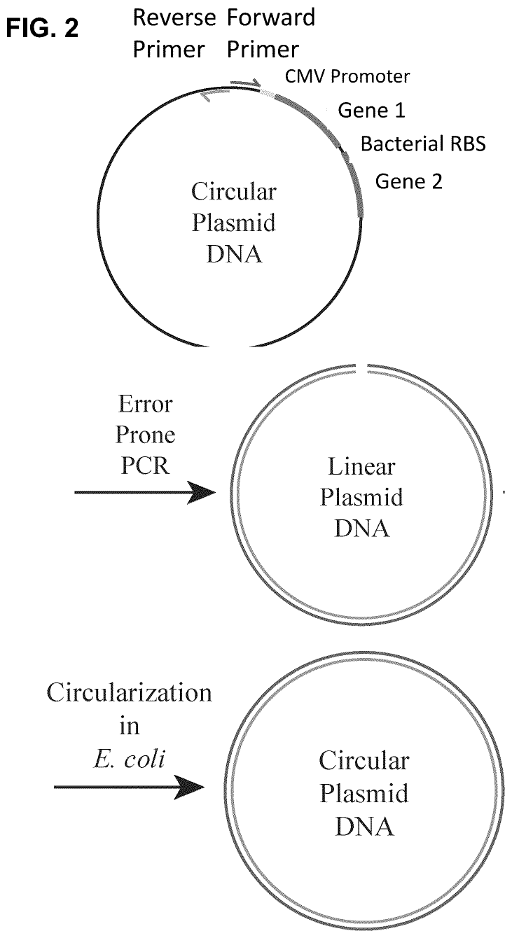

The following materials were used. Primers were purchased from Integrated DNA Technologies to amplify the pDuEx2 vector that allowed expression in bacteria and mammalian cells. The P2EXP-FO forward primer was 5′-GGCCAGATATACGCGTTGACATTG-3′ (SEQ ID NO: 01). The P2EXP-RE reverse primer was 5′-CGTACATCGCGAAGCAGCGC-3′ (SEQ ID NO: 02). The primers were designed to amplify the entire plasmid DNA (FIG. 2). In this configuration, the forward primer was before the CMV promoter to ensure expression from linear and circular plasmid DNA. Primers were changeable to amplify any plasmid DNA. Platinum II Hot-Start Green PCR Master Mix (2×) (ThermoFisher Scientific, 14001012) was used. A DNA Polymerase with editing activity can amplify >10 kB of DNA but will correct DNA mismatches and are unusable for error prone PCR. Standard Taq DNA polymerase can only amplify ˜1-4 kb of DNA due to lack of processivity. The entire plasmid must be amplified using a modified Taq DNA polymerase with a double stranded DNA binding protein (Platinum II Hot-Start DNA Polymerase) to increase processivity and allow for amplification of >7 kb. The Taq DNA polymerase lacks the editing ability to introduce mutations with error prone PCR. Nuclease-free H₂O (typically included in the polymerase kit). 10 mM of each Deoxynucleotide (dNTPs) Solution Mix (New England Biolabs, N0447 L) were used. Higher concentrations of dNTPs can also be used, and the volume can be adjusted accordingly. Magnesium (II) chloride (MgCl₂) (Millipore Sigma, M8266-100G) was used. Manganese (II) chloride Tetrahydrate (MnCl₂·(H₂O)₄) (HiMedia, GRM686-500G) was used. A PCR Thermocycler was used. DpnI (10 U/L) (ThermoFisher Scientific, ER1701) was used. A Zymo DNA Clean & Concentrator-5 Kit (Zymo Research, D4013) was used. A pDuEx2, circular plasmid DNA with Gene 1 and Gene 2 inserts was used. (FIG. 1). A NanoDrop One^{C} UV-Vis spectrophotometer (ThermoFisher, ND-ONEC-W) was used.

In this exemplary experiment, Gene 1 was the small Ultra-Red Fluorescent Protein (smURFP), and Gene 2 was the Synechocystis Heme Oxygenase-1 (HO-1) after a bacterial ribosomal binding site (RBS) (FIG. 2). Gene 1 was expressed in mammalian and bacterial cells. Gene 2 was only expressed in E. coli and produced biliverdin for covalent attachment to smURFP for fluorescence. Biliverdin and biliverdin dimethyl ester are not membrane permeant to E. coli and require cell lysis for chemical addition. An internal ribosomal entry site (IRES) or a self-cleaving peptide (P2A, E2A, F2A, and T2A) can replace the bacterial RBS for the expression of two genes in mammalian cells on a bicistronic mRNA.

Multiple genes can be expressed under different promoters to achieve high, medium, and low expression of different protein genes. Cell-specific promoters allow the expression of genes only in a specific cell type. Inducible promoters allow for temporal control of protein expression. The amplification of the entire plasmid is unique for the evolution of multiple genes simultaneously with a single error prone PCR reaction. The initial plasmid was created using molecular biology, bridging PCR, Gateway cloning, Gibson Assembly Cloning, TOPO TA Cloning, DNA synthesis, or other methods and verified by sequencing. Any plasmid can be usable if the entire plasmid is amplified with the Platinum II Hot-Start DNA Polymerase. Plasmids for bacterial, mammalian, insect, and other organism expression are feasible. As in our example, multiple expression plasmids are useful for continuous fluorescence monitoring in mammalian and bacterial cells. Viral plasmids are amenable to create libraries of viruses for efficient transduction of mammalian cells without electroporation described in this product.

While two proteins are described in the plasmid, other biomolecules can be used to express multiple proteins, RNAs, proteins that synthesize small molecules (HO-1), and any genetically encoded biomolecule. Silencing RNAs can be expressed to knock down gene expression and increase the expression of a protein library to identify phenotypes. Guide RNAs can be expressed for genomic DNA editing in a library of CRISPR/CasX mutants. RNA aptamers may be selected for binding to a protein expressed on the same plasmid. The evolution of proteins to perform complex chemical reactions in buffered solutions and carbon dioxide capture are possible with the proper selection.

Here, 50 μM solutions of the forward and reverse primers were prepared in nuclease-free H₂O. 20 ng/μL solution of circular plasmid DNA was prepared in nuclease-free H₂O. Here, the amount of circular plasmid DNA needed was 20 ng. 20 ng/μL, then add a volume necessary for 20 ng of DNA and then decrease the H₂O volume for a final volume of 50 μL. Generally, large amount of circular plasmid DNA is needed because the error prone PCR is linear and does not appear to be exponential amplification. Increasing the DNA template significantly increases the DNA product. A 275 mM solution of MgCl₂ was prepared in nuclease-free H₂O. Aliquots were created and stored at −20° C. A 10 mM of each dNTPs solution was purchased. Aliquots were created and stored at −80° C. until use. Aliquots were thawed on ice and multiple freeze thaws were avoided.

A 25 mM solution of MnCl₂ was prepared in nuclease-free H₂O. Aliquots were created and stored at −20° C. The diluted primers, DNA, Platinum II Hot-Start Polymerase, dNTPs, MgCl₂, and MnCl₂ were stored on ice. Primers, DNA, MgCl₂, and MnCl₂ solutions can be thawed with hands or water baths, but the prolonged heating of DNA should be avoided. The polymerase chain reaction (PCR) was assembled as described in Table 3 in the order listed. At least three PCR reactions were made to have ≥100 ng linear plasmid DNA for subsequent experiments.

TABLE 3
DNA Library Generation by Error Prone PCR

| Order | Component | Vol. (μL) | Final Conc. |
|---|---|---|---|
| 1 | Nuclease-free H2O | 16 | |
| 2 | Circular Plasmid DNA (10-80 ng) | 1 | 1.2 ng/uL |
| 3 | Forward Primer (50 μM) | 1 | 1 μM |
| 4 | Reverse Primer (50 μM) | 1 | 1 μM |
| 5 | dNTPs (10 mM) | 5 | 1 mM each |
| 6 | MgCl2 (275 mM) | 1 | 7.5 mM |
| 7 | MnCl2 (25 mM) *Add last before | 1 | 0.5 mM |
| | polymerase!* | | |
| 8 | Platinum II Hot-Start Polymerase | 25 | 1.5 mM |
| | Master Mix | | MgCl2 |
| | Final Volume: | 50 | |
| | [symbol] | | |

The MnCl₂ and DNA polymerase should be the last two components added. After the last two additions, the solutions were quickly vortexed, gently centrifuged to get the entire reaction at the bottom of the tube, and the PCR was started as fast as possible. The error prone PCR was performed using the following conditions: 1 cycle (3 min at 95° C.), 35 cycles (30 s at 95° C., 30 s at 63° C., and 1:30 min at 68° C.), 1 cycle (10 min at 68° C.) with a 50 μL reaction volume. After the PCR run was finished, the solution briefly vortexed, and the tube was centrifuged.

The DpnI restriction enzyme and the 10× Tango buffer were placed on ice. Make sure to use the proper buffer that came with the DpnI restriction enzyme. The following ingredients was added to the 50 μL PCR Reaction in this order: 3.0 μL H₂O, 6.0 μL 10× Tango Buffer, then gently vortexed and centrifuged, then 1.0 μL DpnI, and then gently vortexed and centrifuged. The final volume was 60 μL. The volume can be adjusted when the reaction volume is not 50 μL (e.g., buffer volume is 1/10 the final volume and water is added to equal final volume—1.0 μL of DpnI). The reaction was incubated at 37° C. for 2 hours. The DpnI restriction digest removes methylated circular plasmid DNA to avoid selecting the starting DNA. For fluorescent proteins, with the process started dimmer proteins and selected brighter or different wavelengths. Removing the starting DNA reduces the possibility of choosing the same fluorescent protein sequence. The long incubation time ensures multiple cuts of the circular plasmid DNA to avoid recircularization in E. coli.

The PCR product, linear plasmid DNA (FIG. 1), was purified using the Zymo DNA Clean & Concentrator-5 Kit. A 2:1 ratio of DNA binding buffer was added to the PCR product. The solution was transferred to a Zymoclean spin column and centrifuged at 16,000 RCF for 1 min. The flow-through was discarded. The column was washed twice with 200 μL wash buffer (400 μL wash buffer total). The column was centrifuged for 1 min at 16,000 RCF after each wash. Flow-through was discarded. The column was centrifuged for 1 min at 16,000 RCF to remove the excess buffer. The spin column was transferred to a clean 1.5 mL Eppendorf tube and 10 μL of 72° C. H₂O was applied directly to the column matrix. The column matrix was centrifuge at 16,000 RCF for 1 min to elute DNA. The purified linear plasmid DNA was quantified using the NanoDrop One^{C} UV-Vis Spectrophotometer. The yield of linear plasmid DNA after DpnI digestion was typically ˜20-300 ng/μL. Three replicate reactions were performed to ensure 100 ng of linear plasmid DNA for the next step.

Purified, linear PCR DNA (10 μL Linear PCR DNA (˜250 ng/μL), 2.5 μL T4 DNA Polymerase Reaction Buffer, 2.5 μL 10 mM dNTPs, and 7.25 μL H₂O) was prepared. This was gently vortexed and centrifuged. 0.75 μL T4 DNA Polymerase was added. This was gently vortexed and centrifuged. The final volume is 23 μL. This was incubated at 12° C. for 15 minutes. The reaction is stopped with addition of 0.4 μL 0.5 M EDTA and heat inactivate at 75° C. for 20 minutes. The following reaction was created: 4 μL Ligase Buffer, 5 μL Linear PCR DNA with 3′-Adenine overhang removed, and 10 μL H₂O. The reaction was gently vortexed and centrifuged. 1 μL of T4 DNA Ligase was added, gently vortexed, and centrifuged. The final volume was 20 μL. The reaction was incubated at room temperature for 1 hr or for 14° C. for 12 hrs.

In a second experiment, after the quantification, the purified, after the Linear PCR DNA was used as follows: 10.0 μL Linear PCR DNA (˜250 ng/μL), 2.5 μL of T4 DNA Polymerase Reaction Buffer, 2.5 μL of 10 mM dNTPs, and 7.25 μL of H₂O. The solution was gently vortexed and centrifuged. 0.75 μL of T4 DNA Polymerase was added. The solution was again gently vortexed and centrifuged. The final volume was 23 μL. The solution was incubated at 12° C. for 15 minutes. The reaction was stopped with addition of 0.4 μL 0.5 M EDTA and heat inactivated at 75° C. for 20 minutes. The following reaction was created the following reaction. 4 μL of Ligase Buffer, 5 μL of Linear PCR DNA with 3′-Adenine overhang removed, 10 μL of H₂O. This solution was gently vortexed and centrifuged. 1 μL of T4 DNA Ligase was added, which was then gently vortexed and centrifuged. The final volume was 20 μL. The solution was incubated at room temperature for 1 hour or 14° C. for 12 hrs.

### Example 2

### Electroporation of Linear Plasmid DNA in E. coli to Circularize Plasmid DNA

The materials needed for this experiment included the following: 100 ng of purified linear plasmid DNA, Super Optimal broth with Catabolite repression (SOC) media is Hanahan's Broth (SOB medium) (Millipore Sigma, H8032) with 20 mM D-(+)-Glucose (Millipore Sigma, G8270), Ampicillin (Millipore Sigma, A1593), One Shot TOP10 Electrocompetent E. coli (Invitrogen, C664-55), Gene Pulser Xcell Total Electroporation system (BioRad, 1652660), LB (Millipore Sigma, L3522) with 100 μg/mL Ampicillin, 0.2 cm electroporation cuvettes (BioRad, 1652086), QIAprep Spin Miniprep Kit (Qiagen, 27104), and NanoDrop One^{C} UV-Vis Spectrophotometer (ThermoFisher, ND-ONEC-W).

The three reactions were mixed to obtain 100 ng of linear plasmid DNA samples together and the concentration was determined with the NanoDrop One^{C} UV-Vis Spectrophotometer. The volume needed for 100 ng of DNA was calculated. For example, a 34 ng DNA/μL required 2.94 μL for 100 ng DNA. 25 g of the LB broth (Miller's modification) was added to 1 L of dH₂O, which was sterilized by autoclave. It was cooled to room temperature. 2 mL of 50 mg/mL ampicillin stock was added for a final concentration of 100 g/mL ampicillin. It was stored at 4° C. in the cold room. 6 mL of the LB+100 μg/mL ampicillin was added to ten 15 mL Falcon tubes. Frozen SOC medium was thawed by hand and heated to 37° C. in a water bath. A 1.5 mL Eppendorf tube and 0.2 cm electroporation cuvette was placed on ice for ≥10 minutes prior to the electroporation. A One Shot TOP10 electrocompetent E. coli tube was removed from the −80° C. freezer and thawed on ice ≥5 minutes.

The following was done on ice. 100 ng of DNA (obtained in Example 2) was transferred into the chilled Eppendorf tube. 25 μL of thawed electrocompetent bacteria was added. Everything was mixed by gently flicking the tube and then the tube was placed on ice. The mix was not pipetted up and down. Using a long, gel loading pipette tip, the DNA and electrocompetent bacteria solution was transferred to the pre-chilled 0.2 cm cuvette. The solution filled the entire bottom of the cuvette, covered the whole lengths of the electrodes, and there were no bubbles.

The cuvette was removed from ice and the sides were wiped with a Kimwipe to remove moisture. Immediate electroporate was performed with an exponential pulse of 1.6 kV, 200Ω, and 25 μF. If popping was heard and a low time constant was receiving, then arcing occurred. Arcing can be due to moisture on the sides of the cuvette, air bubbles, or solution not covering electrodes. If arcing occurred, then the electroporation was repeated. Then, 250 μL of the warmed SOC was immediately added to the cuvette. The SOC was mixed by pipetting up and down gently. The next steps were done as quickly as possible to avoid bacterial death and lack of growth. 27 μL of bacteria solution was added to each of the ten 15 mL Falcon tubes with 6 mL LB+100 μg/mL. The ten 15 mL Falcon tubes were incubated overnight at 37° C. with shaking at ≥225 rpm.

### Example 3

### E. coli Screening in Liquid Culture and Circular Plasmid DNA Purification

The materials needed for this experiment included the following: UVP ChemStudio 615 Imaging System (Analytik Jena US) or equivalent to image fluorescence of liquid cultures. A plate reader may measure the fluorescence of liquid cultures, a QIAprep Spin Miniprep Kit (Qiagen, 27104), Nuclease-free H₂O, and NanoDrop One^{C} UV-Vis Spectrophotometer (ThermoFisher, ND-ONEC-W).

The tubes were removed from the shaker and fluorescent images of the liquid cultures were captured using a UVP Imager or similar instrument with excitation and emission filters of 644-657 and 660-720 nm, respectively. The exposure time, gain, and light intensity were adjusted for maximum fluorescent intensity. Ensure that the fluorescence signal was separated. These filters used herein were for smURFP and can be changed for other fluorescent proteins.

The circular plasmid DNA were purified from E. coli using QIAprep Spin Miniprep Kit. The 6 mL of overnight bacteria culture was pelleted and centrifuged at 16,000 RCF for 1 min, and the liquid was discarded. 250 μL of Buffer P1 was added and vortexed to resuspend the bacteria cells. All the bacteria were ensured to be suspended, and no bacterial remained on the bottom of the tube or in large aggregates visible by the eye. If the plasmid expressed a fluorescent protein, then the pellet can be colored. For smURFP, this was typically blue. For NIR-FPs, this is usually dark green. The bacteria suspended in Buffer P1 was transferred to a 2 mL Eppendorf tube. 250 μL Buffer P2 was added and the tube was inverted 5 times. 350 L Buffer N3 was quickly added and inverted 5 times to neutralize the pH. This step was done as soon as possible. The tube was not incubated for minutes as 5 minutes was the maximum time allowed. This step was done as quickly as possible.

The tube was centrifuged at 16,000 RCF for 10 min at room temperature. 800 μL supernatant was removed and was added it to the QIAprep 2.0 spin column. Care was taken not to dislodge the pellet, and the supernatant was clear. It was centrifuged at 16,000 RCF for 1 min and the flow-through was discarded. The column was washed with 750 μL Buffer PE and centrifuged at 16,000 RCF for 1 min and then the flow-through was discarded. Centrifuging for 1 min at 16,000 RCF was done to remove the excess buffer. The spin column was transferred to a clean 1.5 mL Eppendorf tube and 50 μL of 72° C. H₂O was applied directly to the column matrix. Again, centrifuging at 16,000 RCF for 1 min was done to elute DNA. Pure water (H₂O) was used to avoid salts and other additives that can inhibit subsequent PCR and sequencing. The circular plasmid DNA concentration was measured using the NanoDrop One^{C} UV-Vis Spectrophotometer.

Equal parts of all ten samples were mixed for a total of 10 μg DNA. To do this, the volume of each sample needed for 1.0 μg of DNA was calculated. After combining the circular plasmid DNA samples, the samples were mixed thoroughly by vortexing and quantified using NanoDrop One^{C} UV-Vis Spectrophotometer. The 10 μg DNA mixture was used as the circular plasmid DNA template for the second round of error prone PCR. Repeating error prone PCR two or more times to increase mutations. After the third round of error prone PCR, the final circular plasmid DNA mixture was used for electroporation in HEK293A cells. In later directed evolution rounds, a single error prone PCR can be performed because multiple mutations should not be necessary to enhance the fluorescence significantly.

### Example 4

### Growing the HEK293A Cells

The materials needed for this experiment included the following: 10 cm Tissue Culture-treated dishes (Eppendorf, 0030702115), Low Glucose (1 g/L) Dulbecco's Modified Eagle's Medium (DMEM) (Millipore Sigma, D6046-1 L), Fetal bovine serum (FBS) (Gibco, 10437-036), 100× Penicillin-Streptomycin (Pen-Strep) (Gibco, 15140-122), 100× GlutaMAX (Gibco, 35050-061), TrypLE Express Enzyme (1×), no phenol red (trypsin) (Gibco, 12604-013), and Dulbecco's Phosphate-Buffered Saline (DPBS) without Ca²⁺ and Mg²⁺ (Gibco, 14190250) with 20 mM HEPES, pH 7.4+1 mM EDTA.

The growth media was made by adding 100 mL of 10% FBS, 10 mL of 1× Pen-Strep, and 10 mL of 1× GlutaMAX directly to 1 L of low glucose (1 g/L) DMEM media bottle to make DMEM 1 g/L glucose+10% FBS+1× Pen/Strep+1× GlutaMax. The sterility of all reagents was ensured by only opening in a biosafety cabinet (BSC). Buffers made in the lab or glassware used were sterilized by filtration or autoclaved before use in cell culture. Low glucose DMEM was used to grow cells slower for less splitting. High glucose (4.5 g/L) DMEM media allowed for faster growth and is used after electroporation.

HEK293A cells were stored frozen in cryovials under liquid nitrogen (N₂) in the cell bank. To start growing HEK293A cells, the Cell Bank binder was consulted to find the cell line, a cryovial was removed out of the liquid N₂ and was quickly placed it in a 37° C. water bath to thaw as fast as possible. Shaking by hand in the water bath helped to thaw faster. While thawing, remember to remove the cryovial taken from the Cell Bank binder. Once thawed, 1 mL was transferred to a 15 mL Falcon tube containing 10 mL of warm growth media.

The cells were pelleted by centrifugation at 90 RCF for 10 minutes and the media was aspirated. This step was necessary to remove the DMSO in the freezing media, small, lysed dead cells, and molecules. 10 mL of fresh, warm growth media was added to the Falcon tube and the cell pellet was resuspended by gently pipetting up and down with a serological pipette. The entire volume was transferred to a 10 cm TC-treated dish and incubated at 37° C. with 5% CO₂ and 95% H₂O. CO₂ was required to maintain the pH at 7.4. Humidity stopped media evaporation. If the phenol red was orange, then the media was acidic. If the phenol red was dark purple, then the media was basic.

Cells were split at 90-100% confluency. Freshly thawed cells grow slower. The cells were checked daily to track the rate of growth and confluency. When the cells were ready to split, DPBS, growth media, trypsin, and a new 10 cm cell dish were placed in the BSC.

The growth media was aspirated from the dish. Aspirating took place from the side of the dish to avoid removing adherent cells. 10 mL of DPBS without Ca²⁺ and Mg²⁺ with 20 mM HEPES pH 7.4+1 mM EDTA was immediately to the dish. 10 mL DPBS was added slowly to the side to avoid removing adherent cells. This solution was necessary to remove divalent cations with EDTA that can inhibit cleavage by trypsin.

The solution was gently mixed in the dish (e.g., typically three times each: clockwise, counter-clockwise, front-to-back, and side-to-side). DPBS was removed by aspiration. 300 μL of TrypLE Express Enzyme was quickly added and mixed by tilting the dish to cover the surface of the dish. The dish was placed into the incubator for ˜5 min. During incubation, 9 mL of fresh growth media was placed in a new, sterile 10 cm TC-treated dish.

Cells were removed from the incubator and 10 mL of fresh growth media was added. The growth media added divalent cations to inhibit trypsin. Usually, the same volume of growth media was added to calculate the dilution. The cells and growth media were pipetted up and down, known as trituration, until there were no more visible clumps of cells or adherent cells. This step was necessary for obtaining single cells to allow room for cell growth.

1 mL of the dissociated cell solution was added to the 9 mL of growth media in a new dish. The solution was gently mixed in the dish, typically three times each (e.g., clockwise, counter-clockwise, front-to-back, and side-to-side). The cells were placed in the incubator (37° C. with 5% CO₂ and 95% H₂O). The remaining cell solution was disposed by aspiration.

At 100% confluency, the 10 cm dishes contained ˜8.8×10⁶ cells. HEK293A cells were electroporated below and require ˜3.0×10⁶ cells per electroporation sample and ˜6.0×10⁵ cells for wild-type HEK293A cells to use as a background sample. If the experiments require electroporating two DNA samples, the ≥6.6×10⁶ cells are necessary. Also, extra cells are preferred in case a dish is contaminated or grows slowly. Two dishes at 100% confluency ensure enough cells on the day of the electroporation. To do this, use 2 dishes. Grow more dishes as necessary.

### Example 5

### Electroporation of HEK293A Cells with Circular Plasmid DNA Library

The materials needed for this experiment included the following: circular plasmid DNA from the third error prone PCR described above, High Glucose (4.5 g/L) DMEM (Gibco, 11965092), 35 mm Tissue Culture-treated Dishes (Eppendorf, 0030700112) or a 6-well plate (Eppendorf, 0030720113), Opti-MEM Reduced Serum Media (Gibco, 31985-070), Countess 3 FL Automated Cell Counter (ThermoFisher, A50299), Countess Cell Counting Chamber Slides (ThermoFisher, C10228), 0.4% Trypan Blue Solution (Gibco, 15250-061), 10× Hanks' Balanced Salt Solution (HBSS) (Gibco, 14065-056), Gene Pulser Xcell Total Electroporation system (BioRad, 1652660), and 0.2 cm electroporation cuvettes (BioRad, 1652086).

Here, the third error prone PCR circular plasmid DNA was electroporated into HEK293A cells for protein expression. Error prone PCR was random, and each reaction generated a unique DNA library that encodes billions of unique biomolecules. No two DNA libraries were the same. The goal of mixing ten error prone PCR samples was to cover as much protein sequence space as possible. All mutations cannot be assessed at one time, and multiple amino acids mutations may be required. For example, for the small Ultra-Red Fluorescent Protein, 20 amino acid mutations were necessary to create a bright, far-red fluorescent protein over 12 selection rounds.

This protocol used electroporation to efficiently introduce DNA into millions of cells, and selection was performed on transiently expressed proteins. The transient expression for 2 days was done to ensure the circular plasmid DNA remains for circular plasmid DNA purification. The procedure could be done by creating viruses using viral plasmids and transducing the mammalian cells with viruses. Viruses would increase the selection time and elevate the biosafety level of the procedure. If using a FACS after transduction with a virus, ensure to use enhanced safety precautions and notify the technician. Many flow core facilities require that the virus be completely gone from samples, which could take 1-2 weeks with additional testing. Recovery of the mutated biomolecule would require mRNA recovery, reverse transcription to ssDNA, PCR amplification to dsDNA, and molecular biology to subclone into a plasmid. Some viruses incorporate viral RNA into the genome as dsDNA, which would require PCR amplification or other means to recover the evolved protein DNA sequence. A biosafety cabinet (BSC) should be used to perform all cell culture work.

After the third error prone PCR from Example 3, the ten circular plasmid DNA libraries from the final PCR were mixed in equal parts. The mixture was 10 μg total and used 1.0 μg of each circular plasmid DNA library with ten libraries. The concentration of the mixed circular plasmid DNA library was measured using the NanoDrop One^{C} UV-Vis Spectrophotometer, and the volume needed for 2 μg of circular plasmid DNA was calculated. 2 mL of high glucose (4.5 g/L) DMEM+10% FBS+1× Pen/Strep+1× GlutaMax was added to six 35 mm cell culture dishes or a 6 well plate and incubated at 37° C. with 5% CO₂ and 95% H₂O. The circular plasmid DNA electroporated into HEK293A cells was divided into five equal 0.4 mL volumes. One dish was used as a control with HEK293A cells without DNA to look at the background fluorescence. Arcing may occur during electroporation and result in a loss of the DNA. Splitting the HEK293A cells into 5 equal volumes avoided the risk of losing all the DNA in a single electroporation.

When the 10 cm TC dishes obtained from Example 4 were 95-100% confluent, the steps set forth in paragraph [0337] (supra) was repeated to remove the adherent HEK293A cells. Parts of Example 4 were repeated. The non-adherent HEK293A cells were transferred to a 15 mL Falcon tube and the cells were pelted by centrifugation at 90 RCF for 5 min. This step was necessary to change the media.

When there were ≥2 dishes of cells, the cells were combined into a 50 mL Falcon tube. Do not use a higher RCF because greater RCF will pellet dead cells and may also kill the living cells. The DMEM+1 g/L glucose+10% FBS+1× Pen/Strep+1× GlutaMax was aspirated completely, and X mL of Opti-MEM (see Example 5) was gently added. Single cell suspensions were created by pipetting up and down with a 5 mL or 10 mL serological pipette or large opening P1000 tip. A serological pipette or a P1000 tip should be used because smaller tips lyse the cells.

The volume of Opti-MEM depended on the confluency of the cells and the desired number of dishes. The concentration was determined by counting the cells using a cell counter (Countess II FL Automated Cell Counter, ThermoFisher). The cell solution should be kept ≥6.0×10⁶ cells/mL, which was the desired final concentration of cells (see Example 6). In a 0.5 mL Eppendorf tube, 5.0 μL of the HEK293A cells were mixed in Opti-MEM with 5.0 μL 0.4% Trypan Blue Solution. 10 μL of cells with trypan blue was added into the cell counter or hemocytometer slide. The concentration of cells/mL was determined. The desired concentration was ≥6.0×10⁶ cells/mL. The volume of Opti-MEM required to dilute the cell solution was calculated to be to 6.0×10⁶ cells/mL. The Opti-MEM was added to the cells and mixed by pipetting.

600,000 cells were required for each 35 mm dish. If the concentration was 6.0×10⁶ cells/mL, then 100 μL was used for each dish. Concentrations slightly greater or less may be used but allowed changing volume greatly because this will change the efficiency of circular plasmid DNA delivery to the HEK293A cells.

In a 1.5 mL Eppendorf tube, 2 μg of circular plasmid DNA from Example 5 was mixed with 100 μL of 6.0×10⁶ cells/mL from Example 5. Mixing occurred via gently flicking or using a P1000 pipette. The entire DNA and cell solution was carefully pipetted using a gel loading tip into a 0.2 cm electroporation cuvette stored at room temperature. It was ensured that the solution filled the entire bottom of the cuvette, covered the whole lengths of the electrodes, and there were no bubbles. The 0.2 cm electroporation cuvette package and lid were not opened outside of a BSC. The cuvette was kept sterile to avoid contamination. When removed from the BSC, the cuvette was capped. The cap was placed on the electroporation cuvette and then removed from the BSC for electroporation. The sides of the cuvette were wiped with a KimWipe and the DNA was electroporated into the HEK293A cells using the Gene Pulser Xcell Total Electroporation system parameters: Gap size=0.2 cm, pulse type is a square wave, pulse amplitude=110 V, and pulse width=40 ms. A square wave pulse was used for mammalian cells. Voltage and pulse width were optimized for efficient DNA delivery. Refer to Bio-Rad Manual and website when using other cell types.

Electroporation was used to achieve high efficiency transfection. Lipofectamine or other polyamine lipid transfections can be used, but this is typically more expensive and requires optimization. Viruses can enhance transduction but require the production of viruses that increase evolution time and requires more stringent safety measures. A loud pop and low time constant are indicative of arcing. Arcing is usually caused by high salt, air bubbles, not covering the entire length of the electrodes, or defective cuvette (rare). Arcing electricity went through the air rather than the solution, and the sample must be thrown out. The solutions are divided into 5 samples to avoid losing all the DNA. If two consecutive samples arc, ensure the parameters and gap size are correct.

The cuvette was wiped with 70% ethanol before placing it into the BSC. A 35 mm cell culture dish or 6-well plate with pre-warmed high glucose (4.5 g/L) DMEM+10% FBS+1× Pen/Strep+1× GlutaMax (from Example 5) was removed from the incubator and placed in the BSC. Using a gel loading tip, the entire electroporated circular plasmid DNA and cell solution was removed and added to the 35 mm cell culture dish. The solution was gently mixed in the dish three times each (e.g., clockwise, counter-clockwise, front-to-back, and side-to-side). The cells were incubated at 37° C. with 5% CO₂ and 95% H₂O. HEK293A cells were initially non-adherent and required time to adhere to the plastic.

The cells were incubated for 2 days to allow biomolecule production for screening. The incubation time may be changed, but this will evolve proteins that require that incubation time for efficient production. Two days is typically the time needed to achieve proper fluorescent protein expression and chromophore attachment to see on any fluorescent microscope. Longer incubations must ensure the circular plasmid DNA is still purified from mammalian cells.

Dead cells typically are non-adherent or floating and may be removed by aspiration. Remember dividing HEK293A cells are also become non-adherent during cell division. If all cells are dead or floating, ensure that you used the high glucose DMEM with GlutaMAX and FBS, had the correct cell number, and cells were added to prewarmed high glucose DMEM. Floating cells may be screened by FACS using side and forward scatter to select properly sized HEK293A cells.

### Example 6

### Fluorescence-Activated Cell Sorting (FACS) of HEK293A Cells with DNA Library

The materials needed for this experiment included the following: EVOS FL Auto II Cell Imaging Epifluorescence Microscope (ThermoFisher, AMAFD2000) or comparable Inverted Fluorescence Microscope with long working distance objectives for imaging through the plastic, Cy5 EVOS LED Light Cube (ThermoFisher, AMEP4956) or equivalent excitation light and filters. For evolving smURFP or Cy5-like fluorescent proteins, Cy5.5 EVOS LED Light Cube (ThermoFisher, AMEP4973) or equivalent excitation light and filters for evolving IFP or Cy5.5-like fluorescent proteins, TrypLE Express Enzyme (1×), no phenol red (Trypsin) (Gibco, 12604-013), Low Glucose (1 g/L) Dulbecco's Modified Eagle's Medium (DMEM) (Millipore Sigma, D6046-1 L), PBS, pH 7.4 (Gibco, 10010-023), 0.5 M EDTA, 1 M HEPES (Gibco, 15630106), FBS (Gibco, 10437036), FACS Transportation Media [(1×PBS, pH 7.4+1 mM EDTA+25 mM HEPES+1% FBS (500 mL 1×PBS+1 mL 0.5 M EDTA+12.5 mL 1 M HEPES+5 mL FBS)], and FACS Recovery Media: [1×HBSS (Gibco, 14065-056)+2 g/L D-(+)-glucose+20 mM HEPES, pH 7.4 (Imaging Media)+10% FBS+1× Pen-Strep (500 mL 1×HBSS+2 g/L glucose+20 mM HEPES, pH 7.4)+50 mL FBS+5 mL 100× Pen-Strep)].

The following protocol was used for the initial screen for far-red and near-infrared fluorescent proteins to confirm cell expression. This protocol, however, can screen protein expression by gel electrophoresis, Western blot, or immunofluorescence. Nucleic acid expression was verifiable by Northern/Southern blot, fluorescence in situ hybridization (FISH), or reverse transcription and PCR amplification with Sanger sequencing or Next Generation Sequencing (NGS). Cell activity or inhibition can be monitored using stable cell lines that express fluorescent sensors for calcium (GCaMP) or other ions. Enzyme sensors, such as kinase and GPCR, can be used to assay the inhibition or activation of the enzymes or receptors. The evolution of biomolecules that reduce gas, such as CO₂, may be performed with CO2 sensors in a closed tissue culture dish. The directed evolution is not limited to fluorescent proteins and can evolve any biomolecules with the appropriate screen.

After 2 days, fluorescent protein expression was verified by fluorescence imaging on an inverted microscope with long working distance or low numerical aperture (NA) objectives for imaging through plastic (EVOS FL Auto 2 with Cy5 and Cy5.5 LED light cubes). Images confirmed fluorescent protein expression and identified spectral properties of fluorescent proteins.

The same exposure, gain, and parameters were used when taking images with the Cy5 and Cy5.5 LED light cubes. A brightfield or white light image was taken to show the total number of cells in the image to calculate the approximate transfection efficiency.

Images were taken using the 10×, 20×, and 40× objectives. The 10× and 20× objectives estimated the transfection efficiency for the entire dish. The 40× objective was useful for publication quality images and identification of the cellular location of fluorescent proteins. The dish lid was not removed so as to keep the cells sterile. Care was taken not to spill media when placing the dish on the microscope. If long working distance objectives are not available, the fluorescence can be screened by adding cells to glass bottom dishes in Example 5. #1.5 glass allowed for imaging on fluorescence microscopes with high numerical apertures and water/oil immersion objectives. Poly-D-Lysine or collagen coating was recommended for adherent cells. Here, MatTek 35 mm glass bottom dishes (P35GC-1.5-14-C) was used.

In the BSC, the media from the 5 dishes was combined in a 15 mL Falcon tube. The step was done to keep all the non-adherent cells that were floating. The floating cells were a mixture of dividing, dead, and non-adherent cells for FACS sorting. Dead cells may be fluorescent and contain circular plasmid DNA. In early selection rounds, more cells were better to increase the amount of circular plasmid DNA purified. The FACS side and front scatter gating will remove small cells and large cell clumps. In later rounds, if most of the cells are non-adherent, or the selection requires the cells to be adherent, the floating cells may be discarded to screen adherent cells. 100 μL of trypsin was added to each dish, the dish was tiled to coat the entire surface evenly, and incubated at 37° C. with 5% CO₂ and 95% H₂O for ˜5 min.

In the BSC, 1 mL of low glucose (1 g/L) DMEM+10% FBS+1× Pen/Strep+1× GlutaMax made in Example 4 was added to stop the Trypsin by the addition of divalent cations. The solution was triturated by pipetting up and down to remove the adherent cells from the dish and break up cell clusters. The cell solution was added to the same 15 mL Falcon tube from Example 6. A serological pipette or a P1000 tip was used to avoid cell lysis.

The cells were pelleted by centrifuge at 90 RCF for 5 min. Media was aspirated carefully so as not to dislodge the pellet. Centrifugation at 90 RCF avoided pelleting small, partial cells and was gentle to prevent killing the cells. The cell pellet was resuspended in 1 mL of prewarmed, 37° C. FACS Transportation Media. The FACS Transportation Media was useful when traveling or keeping the cells outside of an incubator for long periods of time. PBS and HEPES maintained the pH at 7.4 without CO₂. EDTA was added to chelate divalent cations to avoid cell clumping. 1% FBS was added to promote cell health. HEK293A cells survived at room temperature, but delicate cells can be transferred in a heated cooler.

The cells were placed in a Styrofoam secondary container to reduce temperature changes. Gloves, extra FACS Transportation Media to dilute cells, 1.5 mL Eppendorf Tubes with 50 μL of FACS Recovery Media for each selection, P1000 tips, micropipette, and spare 1.5 mL Eppendorf tubes with 50 μL of FACS Recovery Media were gathered. The FACS Recovery Media contains divalent cations, FBS, Glucose, and HEPES to keep the cells alive and avoid DNA degradation after FACS. Penicillin and streptomycin are added to prevent bacterial growth after FACS, which may cause DNA degradation. Most FACS instruments may be contaminated and are not in a BSC. Technicians recommend the addition of Pen/Strep to avoid microbial growth.

Before fluorescence-activated cell sorting (FACS), the cells were mixed by pipetting up and down many times to remove clumps that may have formed during travel. Before FACS, the cells were filtered through a 70 μm nylon mesh (Falcon, 2350) to remove large cell clumps to avoid clogging the FACS tubing and nozzle. Fluorescent proteins were selected using a 637 nm laser with APC or Cy5 filters. Redshifted fluorescent proteins may be selected with a 637 nm laser with APC-Cy5.5 or APC-Cy7 filters. 200,000 cells are run in flow cytometry mode to choose the gate (FIG. 3 and FIG. 4). Typically, from 1-10% of the highest fluorescent cells depending on the total cell number were gated. Ideally, ≥20,000 cells from a selection of 2,000,000 cells are preferred for circular plasmid DNA purification. Collected cells were recorded for each run. Always run ˜200,000 wild-type HEK293A cells to visualize the autofluorescence with identical travel and parameters.

### Example 7

### HEK293A Cell Lysis and Circular Plasmid DNA Recovery

The materials needed for this experiment included the following: a QIAprep Spin Miniprep Kit (Qiagen, 27106), a Zymo DNA Clean & Concentrator-5 Kit (Zymo Research, D4013), Nuclease-free H₂O, and a NanoDrop One^{C} UV-Vis Spectrophotometer (ThermoFisher, ND-ONEC-W). The cells were pelleted by centrifugation at 500 RCF for 5 min. The FACS Recovery Media supernatant was aspirated using a pipette with a P200 tip to ensure total removal and avoid dislodging the pellet. If a small number of cells are in the pellet, and the RCF can be increased slightly to obtain a larger pellet. Avoid high-speed centrifugation that may lyse the cells and lose the circular plasmid DNA in the supernatant. The circular plasmid DNA was purified using the QIAprep Spin Miniprep Kit. The HEK293A cell pellet was resuspended in 250 μL Buffer P1 and vortex. Care was taken to ensure the pellet was dislodged, and the solution was homogenous.

250 μL Buffer P2 was added and mixed by inverting 5 times. 350 μL Buffer N3 was quickly added and inverted 5 times to neutralize the pH. This step was done as soon as possible. Do not incubate for 5 minutes. 5 minutes was the maximum time allowed and care was taken to do this as fast as possible. The solution was then centrifuged at 16,000 RCF for 10 min at room temperature. 800 μL supernatant was removed and added to the Zymoclean Spin column. Care was taken not to dislodge the pellet, and to keep the supernatant clear. Centrifuging at 16,000 RCF for 1 min was done and flow-through was then discarded. Make sure to use the Zymoclean Spin column, not the QIAprep Spin column. All buffers used in the rest of the purification process were from the Zymo DNA Clean & Concentrator-5 Kit.

The column was twice washed with 200 μL wash buffer (400 μL wash buffer total), and then centrifuged for 1 min at 16,000 RCF after each wash. The flow-through was discarded. Again, centrifuging for 1 min at 16,000 RCF was done to remove the excess buffer. The spin column was transferred to a clean 1.5 mL Eppendorf tube and 10 μL of 72° C. dH₂O was applied directly to the column matrix. Centrifuging at 16,000 RCF for 1 min was used to elute DNA. Pure, nuclease-free water (H₂O) was used to avoid salts and other additives that may inhibit subsequent PCR and sequencing. The purified circular plasmid DNA was quantified using the NanoDrop One^{C} UV-Vis Spectrophotometer. The total amount of DNA recovered was ˜30-50 ng. The volume of solution needed for 10 ng of circular plasmid DNA purified from HEK293A cells was calculated.

### Example 8

### Electroporation of E. coli and Plating on LB/Agar with Ampicillin Plates

The materials needed for this experiment included the following: Ampicillin (Millipore Sigma, A1593), Luria broth (LB) agar (Millipore Sigma, L2897) 10 cm plates with 100 μg/mL Ampicillin, Super Optimal broth with Catabolite repression (SOC) media is Hanahan's Broth or SOB medium (Millipore Sigma, H8032) with 20 mM D-(+)-Glucose (Millipore Sigma, G8270), One Shot TOP10 electrocompetent E. coli (ThermoFisher, C66455), Gene Pulser Xcell Total Electroporation system (BioRad, 1652660), and 0.2 cm electroporation cuvettes (BioRad, 1652086).

Gene expression of β-lactamase appears upregulated after purification of circular plasmid DNA from HEK293A cells. A single colony expresses enough β-lactamase to remove ampicillin for surrounding, untransformed bacteria to survive. Before starting, the LB/agar plates with ampicillin were prepared at least one day prior. Extra plates for plating were poured and were stored. Extra plates can be stored for >6 months at 4° C. in the dark. Each mutagenic DNA library required ˜20 LB/agar plates.

The number of plates changes depending on the number of bacterial colonies. Prepare the plates by removing the pre-autoclaved, 500 mL LB/agar jar from the refrigerator. The top was unscrewed and was placed on top to avoid pressure buildup and LB/agar boiling into the microwave. Microwaving occurred for 30 see and the LB/agar mixture was swirled until fully dissolved. The dissolved LB/agar jar was put into a 55° C. water bath for ˜45 min. This step was performed to cool the mixture to 55° C. for ampicillin addition and avoided agar solidification.

The LB/agar jar can be left longer than 45 min in the water bath. The temperature was checked at 55° C. with an infrared thermometer. 100 μg/mL ampicillin from 50 mg/ml ampicillin stocks was added. For a 500 mL LB/agar jar, add 1 mL of the 50 mg/mL ampicillin stock solution. The solution was mixed thoroughly by swirling. Care was taken not to shake vigorously to avoid air bubbles.

The LB/agar jar were stored at 55° C. to avoid agar solidification. A 500 mL LB/agar jar was enough for a sleeve of 20 plastic bacteria plates. The LB/agar mixture was carefully poured into the plates to avoid creating bubbles. This step was done near an open flame to avoid contaminants in the air. The plates were left at room temperature and covered to avoid ampicillin degradation by light.

The frozen SOC was warmed by hand until completely thawed. The SOC was heated in a 42° C. dry bath block and remained in the dry bath block until needed. A 1.5 mL Eppendorf and a 0.2 cm electroporation cuvette was placed on ice for ≥10 minutes prior to electroporation. A single tube of One Shot TOP10 electrocompetent E. coli from the −80° C. freezer was placed on ice ≥5 minutes before electroporation. The electrocompetent E. coli was fully thawed before use. Care was taken not to thaw by pipetting up and down. The tube was quickly flicked to gently mix but avoid a temperature change.

The following steps were done on ice. 10 ng of circular plasmid DNA purified from HEK293A cells from Example 7 was transferred into the chilled Eppendorf tube and 15 μL of freshly thawed electrocompetent bacteria was added. Pipetting was not up and down. Rather, the solution was mixed by gently flicking the tube and the tube was placed on ice.

A gel loading pipette tip was used to transfer the circular plasmid DNA and bacteria solution to the pre-chilled 0.2 cm cuvette. Care was taken to ensure the solution filled Quickly added the whole length of the electrodes, and there were no bubbles. Both sides of the cuvette were wiped with a KimWipe to remove moisture. If the cuvette is wet, arcing may occur during electroporation. The cuvette was placed into the electroporation system and electroporated with an exponential pulse with voltage=1.6 kV, resistance=200Ω, and capacitance=25 uF. An exponential pulse is used to electroporate circular plasmid DNA into bacteria. A loud pop and low time constant indicate arcing, and the electricity did not flow through the sample. Discard the tube and repeat the electroporation.

250 μL of prewarmed SOC was immediately added to the cuvette and mixed by pipetting up and down slowly. The bacteria in SOC was transferred to a Falcon tube and incubated at 37° C. shaking at 225 rpm for 1 hour. The bacteria in SOC were spread on the LB/Agar plates with 100 μg/mL ampicillin using sterile, autoclaved glass beads in the following amounts: 2 plates with 40 μL, 5 plates with 20 μL, 8 plates with 10 μL, 5 plates with 1 μL, for a total of 20 plates. Care was taken to spread all the bacteria and SOC solution. Different volumes are necessary to ensure that the plates are not overgrown with a lawn or tiny bacteria colonies. Ensure there is enough space to select a single colony to avoid mixing the circular plasmid DNA from two or more bacterial colonies. The purified circular plasmid DNA from each evolution round has different transformation efficiencies, and the dilutions ensure plates will contain individual colonies. The plates were incubated overnight at 37° C. in a dry incubator with the LB/Agar side facing up.

### Example 9

### Single Bacteria Colony Screening, Selection, and Growth for DNA Purification

The materials needed for this experiment included the following: UVP ChemStudio 615 Imaging System (Analytik Jena US) or equivalent to image fluorescence of liquid cultures. A plate reader may be used to measure the fluorescence of liquid cultures, LB liquid media with 100 μg/mL ampicillin, QIAprep Spin Miniprep Kit (Qiagen, 27104), and Nuclease-free H₂O, NanoDrop One^{C} UV-Vis Spectrophotometer (ThermoFisher, ND-ONEC-W).

Images of the bacterial colonies were captured using a UVP Imager or similar instrument with excitation and emission filters of 644-657 and 660-720 nm, respectively. The exposure time, gain, and light intensity was adjusted for maximum fluorescent intensity and to ensure that the fluorescence signal was not saturated.

These filters are for smURFP and are changeable for other fluorescent proteins. The images were opened in NIH ImageJ (Schindelin J, et al. (2012) Nat Methods. 9:676-682; Schneider C A, et al. (2012) Nat. Methods. 9:671-675) to select the brightest fluorescent colonies. The “Brightness/Contrast” was used to adjust the image histogram or use the “Find Maxima” function and adjust the threshold value until 10-15 colonies were marked as maxima to locate the brightest fluorescent colonies. The selection is in mammalian cells, and the brightest fluorescent proteins in mammalian cells may be dimmer in bacterial cells. Pick an equal amount of bright and less fluorescent single colonies to avoid evolving fluorescent proteins that fluorescence well in bacteria.

The selected colonies were picked using a pipette tip or autoclaved toothpick. A single colony was carefully touched and the tip was placed into a 15 mL Falcon tube. A separate tube was used for each colony. 6 mL of LB with 100 μg/mL ampicillin was added to each 15 mL Falcon tube and incubated at 37° C. with shaking at ≥225 rpm overnight. After overnight incubation, part of Example 3 was repeated to fluorescently image the liquid cultures and purify the circular plasmid DNA. The concentration of circular plasmid DNA was quantified using the NanoDrop One^{C} UV-Vis Spectrophotometer.

### Example 10

### Circular Plasmid DNA Lipofection into HEK293A Cells and Fluorescence Screening

The materials needed for this experiment included the following: Lipofectamine 3000 (ThermoFisher, L3000-015), 96-Well Black/Clear Bottom Plate, TC Surface (Thermo Fisher Scientific, 165305), HEK293A Cells (ThermoFisher, R70507), Opti-MEM (Gibco, 31985-070), Biliverdin dimethyl ester (BVMe2) (Frontier Scientific, B610-9), Imaging Media: 1×HBSS (Gibco, 14065-056)+2 g/L D-(+)-glucose+20 mM HEPES, pH 7.4, and Varioskan LUX Fluorescence Plate Reader (ThermoFisher).

HEK293A cells were grown to 95-100% confluency and follow some steps in Example 4. 200 μL of HEK293A cell solution was added to a well in a 96-well plate. This process was continued to match twice the number of purified circular plasmid DNA samples from Example 9. 9-12 extra wells were added in case a well did not grow and to have control wells without DNA and with starting fluorescent protein DNA (discussed below) for background autofluorescence. The cells were allowed adhere overnight with incubation at 37° C. with 5% CO₂ and 95% H₂O.

2 wells for each purified DNA were obtained from Example 9. If there are 15 DNA samples, 30 wells are used. An additional 9-12 wells of control cells, including 3 wells transfected with the original smURFP circular plasmid DNA without the addition of BVMe₂, 3 wells transfected with the original smURFP circular plasmid DNA with the addition of 1 μM BVMe₂, and 3 wells with HEK293A cells without DNA transfection. The minimum number of wells is 9, and the extra 3 may substitute if another well does not adhere. For this experiment, 39 wells are necessary, with 3 additional wells.

After overnight incubation, the adherent HEK293A cells were transfected with DNA using lipofection following the Lipofectamine 3000 protocol. All steps were performed in the BSC to keep solutions and cells sterile. For each well, two (2) 1.5 mL Eppendorf tubes were used. 100 ng of circular plasmid DNA was mixed with 5 μL Opti-MEM and 0.2 μL P3000 in a 1.5 mL tube. In the second 1.5 mL Eppendorf tube, 0.15 μL Lipofectamine 3000 was mixed with 5 μL Opti-MEM. The solutions were vortexed separately and the two tubes were combined to form DNA complexes with polyamine lipids. The mixture was incubated for 15 minutes in the BSC without UV light. When the user leaves the room, the “No UV sign” was placed on the BSC. All the circular plasmid DNA complexed with polyamine lipids solution was carefully added to each well by using the side of the well. Care was taken to avoid dislodging the adherent cells from the bottom of the well. Incubation lasted overnight at 37° C. with 5% CO₂ and 95% H₂O.

There were 2 replicates for each circular plasmid DNA sample, and the volumes should be doubled to 200 ng circular plasmid DNA+10 μL Opti-MEM+0.4 μL P3000. A stock solution of Opti-MEM and Lipofectamine 3000 should be made for all wells in a single tube. For 39 wells, add 5.85 μL of Lipofectamine 3000 to 195 μL of Opti-MEM. Remember to mix components well.

Do not close the BSC glass shield. Closing the shield will activate the UV light, which will destroy the DNA. If you leave the hood during the incubation, place a “No UV sign” on the glass shield. After 24 hr, for the HEK293A cells expressing smURFP, the media was aspirated and replaced with 0.2 mL of 1 μM BVMe2 in growth media (2 mL DMEM+10% FBS+1× Pen/Strep+1× GlutaMax+0.2 μL of 5 mM BVMe2 in DMSO). Incubation was done overnight at 37° C. with 5% CO2 and 95% H2O. The DMSO was dense and sunk to the bottom of the tube. The solution was mixed well. After 24 hr, the media was aspirated from the wells and replaced with 200 μL of imaging media (1×HBSS (Gibco, 14065-056)+2 g/L D-(+)-glucose+20 mM HEPES, pH 7.4). Imaging media was added to 3 unused wells to measure autofluorescence of the growth media. Care was taken not to aspirate the cells off the bottom of the wells. The HBSS was gently added to the side of the well. Adding to the side avoided dislodging adherent cells.

Imaging media was used to maintain pH=7.4 without CO₂. Glucose was added to ensure healthy cells. This media was suitable for 3 hr-5 hr. Longer incubations may be toxic to cells, and other buffers should be used for long-term imaging.

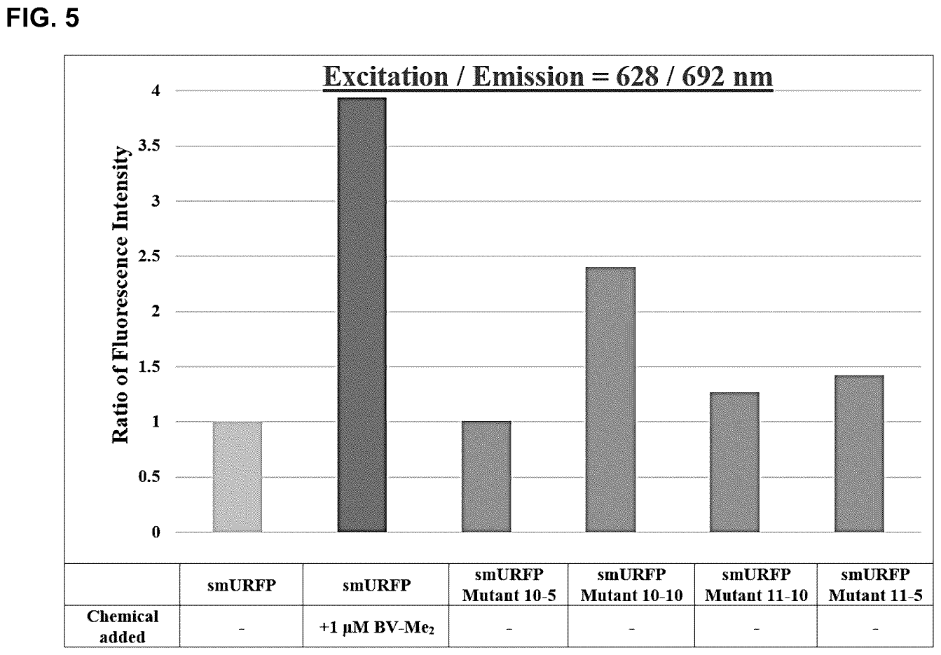

The cells were fluorescently image as described above in Example 6. The Varioskan LUX Fluorescence Plate Reader (ThermoFisher) was used to automatically screen the fluorescence of the transiently expressed fluorescent proteins in HEK293A cells (FIG. 5). The average fluorescence and absorbance of adherent HEK293A cells was measured using the following parameters: Fluorescence 1: Excitation 642 nm & Emission 672 nm (far-red fluorescence); Fluorescence 2: Excitation 685 nm & Emission 715 nm (near-infrared fluorescence); Excitation bandwidth: 12 nm for both; Optics: Read from the bottom (Record from adherent cells); Dynamic range: Low; Measurement time: 1000 ms; Measurement points: All points on well. Absorbance 1: 280 nm, Absorbance 2: 380 nm, Absorbance 3: 642 nm; Use transmittance: No, Pathlength correction: Yes, Measurement time: 1000 ms. Background subtraction was selected for every measurement taken.

All the wells were labeled properly to perform background subtraction automatically. New circular plasmid DNA samples were “Unknown”, wells with untransfected HEK293A cells, wells with HEK293A cells expressing smURFP with 1 μM BVMe₂, HEK293A cells expressing smURFP with 1 BVMe₂ were “Controls”, and the wells with imaging media alone were the “Background”. After the plate reader finished the measurements, the 96-well black plate was placed in a BSC. The media was aspirated from the wells and the 96-well black plate was sprayed with 70% ethanol to sterilize the wells. Care was taken to avoid spraying ethanol in unused wells, which may be used in future experiments.

The average fluorescence measurements were compared at both wavelengths to the HEK293A cells expressing smURFP (controls) to select the circular plasmid DNA that produced fluorescence brighter than smURFP without BVMe₂. The epifluorescence images were compared to each other and against the control images. DNA expressing fluorescent proteins with the highest average fluorescence were selected for Sanger sequencing. The plate reader was used to verify the FACS selection. A single cell shows highly variable fluorescence, and this screen measured the fluorescence from many cells to get an average fluorescence that is more accurate.

Purified circular plasmid DNA was selected and sent for Sanger sequencing to GENEWIZ. Circular plasmid DNA was diluted to a concentration of 80 ng/μL DNA in 10 μL in nuclease-free H₂O. GENEWIZ primers were used to sequence from the CMV promoter (pDuEx2 and pCDNA3). Sequencing was usually 800 bp-1,000 bp. Design custom primers with 20-30 bases before the desired sequencing region because the start of the fluorescent chromatograph was generally saturated with fluorescence from the initial PCR terminations.

### Example 11

### DNA Sequencing and Analysis to Determine Amino Acid Mutations

The following protocol analyzes the Sanger sequencing to determine amino acid changes in the newly produced biomolecules. The DNA nucleotide mutations are translated into protein and compared to the parent protein to identify amino acids changes that increase fluorescence. Analyze selected circular plasmid DNA and only select fluorescent proteins that have unique and multiple amino acid changes for subsequent directed evolution. Do not select DNA with mutations in non-coding regions or silent mutations that do not change the amino acid. If there are no mutations, select non-coding and silent mutations over the initial DNA. These mutations may enhance transcription or translation and increase the probability of amino acid change in subsequent rounds. This protocol describes the method for selecting the fluorescent protein used as the template in the error prone PCR for the next round of directed evolution.

A Plasmid Editor (APE) made by M. Wayne Davis is freely available at https://jorgensen.biology.utah.edu/wayned/ape/or use a similar DNA analysis software to align the new DNA and protein sequences. The mutated nucleotides in the new fluorescent proteins were located using the starting protein and PCR template alignment. Highlighting or case changing was used to locate the mutated nucleotide. All the genes were translated to obtain the amino acid sequences. The mutated nucleotide in the new fluorescent protein amino acid sequence was identified.

Whether the nucleotide mutations changed the amino acid or were “silent mutations” due to the redundancy of the genetic code was determined. Silent mutations are undesired because the amino acid sequence remains the same, but the nucleotide change may enhance protein expression. Desirable nucleotide mutations cause an amino acid change. Error prone PCR is biased towards specific nucleotides, so if there are no amino acid changes, you should perform multiple rounds of error prone PCR to enhance the mutation frequency. Adding high concentrations of dNTPs with high concentrations of Mg²⁺ and Mn²⁺ can also increase the frequency of mutations. However, increasing the concentration of these components usually decreases the overall yield of the PCR.

The new fluorescent proteins with unique and multiple amino acid changes were selected as the template for the next round of directed evolution. Multiple fluorescent protein sequences may be mixed but use in equal proportions to avoid bias in the library. If there are no amino acid changes, error prone PCR should be repeated with multiple PCR reactions to enhance the mutation frequency. If the fluorescent protein sequences have silent mutations, use these as templates for the error prone PCR because these increase the probability of changing the amino acid in subsequent rounds.

What is claimed is:

1. A system for directed evolution of one or more biomolecules of interest, the system comprising:
a DNA polymerase fused to a DNA binding protein, wherein the DNA polymerase lacks editing activity; and,
a plasmid comprising one or more promoters for transcription and translation in a host cell, and one or more polynucleotide sequences encoding one or more biomolecules of interest to be evolved,
wherein the system evolves the one or more biomolecules of interest by introducing one or more mutations into the one or more polynucleotide sequences encoding the one or more biomolecules of interest.

2. The system of claim 1, wherein the DNA polymerase fused to a DNA binding protein amplifies the plasmid in its entirety.

3. The system of claim 1, wherein the DNA polymerase fused to a DNA binding protein amplifies about 0.1 kilobases of DNA to about 15 kilobases of DNA.

4. The system of claim 1, wherein the plasmid comprises two biomolecules of interest to be evolved.

5. The system of claim 1, wherein the one or more biomolecules of interest comprises a nucleic acid, an oligonucleotide, a protein, a peptide, an enzyme, an amino acid, an organic molecule, a small molecule, or any combination thereof.

6. The system of claim 1, wherein the biomolecule of interest comprises RNA.

7. The system of claim 6, wherein the RNA comprises a silencing RNA or a guide RNA.

8. The system of claim 7, wherein the silencing RNA knocks down gene expression and/or increases the expression of a protein implicated in a disease or disorder.

9. The system of claim 1, wherein the biomolecule of interest comprises a full-length antibody, an antigen binding fragment of a full-length antibody, a Fab fragment, a single chain antibody (scFv), a diabody, a triabody, a minibody, a nanobody, a single-domain antibody, camelids, or any combination thereof.

10. The system of claim 1, wherein the biomolecule of interest comprises an enzyme that can perform novel protein, RNA, and/or DNA modifications, or can perform a combination of novel protein, RNA, and/or DNA modifications.

11. The system of claim 1, wherein the biomolecule of interest comprises an enzyme for degrading plastic or waste products, thereby forming useful molecules, or an enzyme for degrading carbon dioxide or other gases to create synthetically useful molecules and reduce greenhouse gases.

12. The system of claim 1, wherein the biomolecule of interest improves disease resistance, increases and/or enhances immunity to pests and/or pathogens, improves yield, increases and/or enhances tolerance to abiotic stress, increases and/or enhances tolerance to drought, increases and/or enhances resistance to one or more herbicides, increases and/or enhances photosynthesis or the rate of photosynthesis, improves biomass, increases and/or enhances nutritional content, improves tolerance to temperature, ultraviolet radiation, and/or other abiotic factors, or any combination thereof, in a crop of interest or a crop having commercial, economic, and/or social value.

13. The system of claim 1, wherein the plasmid comprises a bacterial expression plasmid, a mammalian expression plasmid, an insect expression plasmid, a viral expression plasmid, a species-specific host organism expression plasmid, or any combination thereof.

14. The system of claim 1, wherein the mutation efficiency comprises about 1-in-400 base pairs, about 1-in-350 base pair, 1-in-300 base pairs, 1-in-250 base pairs, 1-in-200 base pairs, 1-in-150 base pairs, or 1-in-100 base pairs.

15. The system of claim 1, wherein the host cell comprises a bacterial cell, a yeast cell, an insect cell, a mammalian cell, a species-specific host cell, or any combination thereof.

16. A method of directed evolution of one or more biomolecules of interesting comprising using the system of claim 1.

17. The method of claim 16, comprising preparing a DNA library by performing error prone PCR mutagenesis; circularizing plasmid DNA by electroporating linear plasmid DNA in bacteria; screening the bacteria and purifying circular plasmid DNA; growing mammalian cells; electroporating the grown mammalian cells with the circular plasmid DNA; subjecting the electroporated mammalian cells to cell sorting; lysing the sorted mammalian cells and recovering circular plasmid DNA; electroporating bacteria and plating electroporated bacteria; lipofecting circular plasmid DNA into mammalian cells and screening lipofected mammalian cells; lipofecting circular DNA into mammalian cells and screening lipofected mammalian cells; and identifying amino acid mutations by sequencing and analyzing DNA.

18. The method of claim 17, comprising repeating the error prone PCR mutagenesis one or more times.

19. The method of claim 17, wherein the mammalian cells are HEK293A cells.

20. The method of claim 17, wherein the method uses less than 5 hours of hands-on experiment time.

21. The method of claim 17, wherein the method eliminates about 70% of hands-on experiment time when compared to a traditional method of directed evolution.

22. The method of claim 17, wherein the method does not comprise use of a virus or a viral vector.

23. The method of claim 16, wherein the method does not comprise use of a virus or a viral vector.


SEQUENCE LISTING (2 sequences)

SEQ ID NO: 1 - DNA, 24 bp, synthetic construct
  source 1..24: mol_type = other DNA
ggccagatatacgcgttgacattg

SEQ ID NO: 2 - DNA, 20 bp, synthetic construct
  source 1..20: mol_type = other DNA
cgtacatcgcgaagcagcgc